(19) United States
(12) Patent Application Publication
(10) Pub. No.: US 2026/0002206 A1
(43) Pub. Date: Jan. 1, 2026

(54) Combination Panel Cell-Free DNA Monitoring

(71) Applicant: Seattle Project Corp., Dover, DE (US)

(72) Inventors: Desiree Schenk, Belmont, MA (US); Matthew Joseph Davis, Scituate, MA (US); Oliver Spiro, Northampton, MA (US)

(21) Appl. No.: 19/194,819

(22) Filed: Apr. 30, 2025

Related U.S. Application Data
(63) Continuation of application No. PCT/US2023/036536, filed on Oct. 31, 2023.
(60) Provisional application No. 63/381,747, filed on Oct. 31, 2022.

(51) Int. Cl. C12Q 1/6874; C12Q 1/6806; C12Q 1/6886

(52) U.S. Cl. CPC C12Q 1/6874; C12Q 1/6806; C12Q 1/6886; C12Q 2600/106; C12Q 2600/156

(57) ABSTRACT

Methods and compositions for monitoring mutation burden, cancer status, vaccine efficacy using cell-free DNA sequencing following enrichment with combination probe panels are disclosed.

### CROSS REFERENCE TO RELATED APPLICATIONS

This application is a continuation of International Patent Application No. PCT/US2023/036536, filed Oct. 31, 2023, which claims the benefit of, and priority to, U.S. Provisional Application No. 63/381,747, filed Oct. 31, 2022, which is hereby incorporated in its entirety by reference.

### SEQUENCE LISTING

The instant application contains a Sequence Listing which has been submitted via Patent Center and is hereby incorporated herein by reference in its entirety. The accompanying sequence listing .XML file name SEP-118USWOC1, was created on Sep. 2, 2025, and is 8,449 bytes in size.

### BACKGROUND OF THE INVENTION

Therapeutic vaccines based on tumor-specific antigens hold great promise as a next-generation of personalized cancer immunotherapy. For example, cancers with a high mutational burden, such as non-small cell lung cancer (NSCLC) and melanoma, are particularly attractive targets of such therapy given the relatively greater likelihood of neoantigen generation. Early evidence shows that neoantigen-based vaccination can elicit T-cell responses and that neoantigen targeted cell-therapy can cause tumor regression under certain circumstances in selected patients.

One question for neoantigen vaccine design is which of the many coding mutations present in subject tumors can generate the “best” therapeutic neoantigens, e.g., antigens that can elicit anti-tumor immunity and cause tumor regression. Targeting antigens that are shared among patients with cancer hold great promise as a vaccine strategy, including targeting both neoantigens with a mutation as well as tumor antigens without a mutation (e.g., tumors antigens that are improperly expressed).

Challenges with shared antigen vaccine strategies include at least monitoring cancer status and/or efficacy of a vaccine prior to or following administration of a cancer vaccine to a subject. For example, many standard methods to monitor disease that are invasive or burdensome, such as radiological assessments (e.g., CT scans) or tumor biopsies. In addition, certain existing cell-free DNA monitoring methods suffer from reduced monitoring capability of cancer status and burden, such as reduced monitoring sensitivity, as they only monitor a small fraction of mutations (e.g., less than 50) associated with a tumor exome. Likewise, certain existing cell-free DNA monitoring methods (e.g., Wan et al.; Science Translational Medicine 17 Jun. 2020: Vol. 12, Issue 548) suffer from reduced accuracy and reliability as they only monitor greater numbers of mutations at low-sequencing depth.

To monitor cfDNA, assays are generally stratified into those that are tumor-naïve and tumor-informed. Tumor-naïve monitoring utilizes a panel approach where DNA targets are fixed and tend to capture only a few variants from many patients. Tumor-informed approaches rely on sequencing a biopsy and longitudinally tracking a set of defined, individualized variants over time, but generally monitor a smaller footprint. To overcome the smaller footprint of fixed gene sets and individualized panels, WES and whole genome sequencing (WGS) of liquid biopsy samples provide expanded breadth amenable to de novo variant discovery or detection without the need for tissue that can be used for either early detection or recurrence. However, the expanded breadth of coverage generally has increased costs that make these techniques impractical in a clinical setting and/or requires a lower overall sequencing depth to maintain costs and use of different bioinformatics strategies.

Accordingly, needed in the field are accurate, reliable, and less invasive cancer monitoring methods, such as cell-free DNA sequencing methods that offer broad target coverage (e.g., at least 95% of mutations present in a cancer exome) at high sequencing read depth (e.g., at least 1000×). Also needed are compositions and methods that can effectively monitor subject-specific efficacy as well as broader monitoring capabilities, including monitoring tumor evasion mutations.

### SUMMARY OF THE INVENTION

Provided for herein is a panel of polynucleotide probes for enriching cfDNA, the panel comprising: (A) one or more tumor-informed polynucleotide probes; and (B) one or more tumor-naïve polynucleotide probes.

In some aspects, the one or more tumor-informed polynucleotide probes are configured to capture a target sequence comprising an epitope sequence encoded by a cancer vaccine administered to a subject, wherein the subject has been determined to have a tumor expressing the epitope sequence. In some aspects, the KRAS mutation is selected from the group consisting of a KRAS_G12C mutation, a KRAS_G12D mutation, a KRAS_G12V mutation, and a KRAS_Q61H mutation. In some aspects, the epitope sequence comprises a mutation selected from the group consisting of: KRAS_G13D, KRAS_Q61K, TP53_R249M, CTNNB1_S45P, CTNNB1_S45F, ERBB2_Y772_A775dup, KRAS_G12D, KRAS_Q61R, CTNNB1_T41A, TP53_K132N, KRAS_G12A, KRAS_Q61L, TP53_R213L, BRAF_G466V, KRAS_G12V, KRAS_Q61H, CTNNB1_S37F, TP53_S127Y, TP53_K132E, and KRAS_G12C. In some aspects, the epitope sequence comprises an EGFR mutation. In some aspects, the EGFR mutation comprises an EGFR_L858R mutation.

In some aspects, the epitope sequence comprises one or more subject-specific epitopes, wherein the tumor of the subject has been sequenced to determine the subject-specific epitopes to be encoded by the cancer vaccine. In some aspects, the one or more subject-specific epitopes comprises at least 2 subject-specific epitopes, at least 10 subject-specific epitopes, at least 20 subject-specific epitopes, or between 2-20 subject-specific epitopes. In some aspects, the one or more subject-specific epitopes comprises between 2-20 subject-specific epitopes.

In some aspects, the panel further comprises additional tumor-informed polynucleotide probes that capture additional target sequences, wherein the tumor has been determined to express the additional target sequences, and wherein the additional target sequences are not encoded by the cancer vaccine. In some aspects, the additional target sequences comprise at least 10 target sequences, at least 20 target sequences, at least 30 target sequences, at least 100 target sequences, between 10-500 target sequences, between 30-500 target sequences, between 100-500 target sequences, between 10-100 target sequences, between 30-100 target sequences, or between 100-100 target sequences. In some aspects, the additional target sequences have been predicted to be presented by at least one HLA of the subject.

In some aspects, the one or more tumor-naïve polynucleotide probes are configured to capture a target sequence comprising a sequence of interest selected from the group consisting of: a cancer-associated gene, an oncogene, a tumor-suppressor gene, an interferon-y signaling pathway gene, an antigen-processing pathway gene, and combinations thereof.

In some aspects, the one or more tumor-naïve polynucleotide probes are configured to capture a target sequence comprising a sequence of interest selected from each of a cancer-associated gene, an oncogene, a tumor-suppressor gene, an interferon-y signaling pathway gene, and an antigen-processing pathway gene.

In some aspects, the cancer-associated gene is selected from the group consisting of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2. In some aspects, the cancer-associated gene comprises each of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2.

In some aspects, the oncogene is selected from the group consisting of: ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20. In some aspects, the oncogene comprises each of: ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20.

In some aspects, the tumor-suppressor gene is selected from the group consisting of: TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3. In some aspects, the tumor-suppressor gene comprises each of: TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3.

In some aspects, the interferon-y signaling pathway gene is selected from the group consisting of: IFNGR1, INFGR2, JAK1, JAK2, and STAT1. In some aspects, the interferon-y signaling pathway gene comprises each of: IFNGR1, INFGR2, JAK1, JAK2, and STAT1.

In some aspects, the antigen-processing pathway gene is selected from the group consisting of: B2M, HLA-A, HLA-B, HLA-C, HLA-E, TAP1, TAP2, NLRC5, CALR, CANX, PSMB2, and TAPBP. In some aspects, the antigen-processing pathway gene comprises each of: B2M, HLA-A, HLA-B, HLA-C, HLA-E, TAP1, TAP2, NLRC5, CALR, CANX, PSMB2, and TAPBP.

In some aspects, the one or more tumor-naïve polynucleotide probes are configured to capture a target sequence comprising a sequence of interest selected from the group consisting of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2, TAPBP, and combinations thereof.

In some aspects, the one or more tumor-naïve polynucleotide probes are configured to capture a target sequence comprising a sequence of interest selected from each of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2, and TAPBP.

In some aspects, the one or more tumor-naïve polynucleotide probes are configured to capture a target sequence comprising a sequence of interest selected from each of: ABL1, AKT2, ALK, APC, AR, ATR, ATRX, BARD1, BCL6, BMPR1A, BRAF, BRCA1, BRCA2, BTK, CARD11, CCND1, CCND3, CDK12, CFH, CREBBP, CTNNB1, DDR2, DNMT3A, EGFR, EP300, ERBB2, ERBB3, ERCC2, ERCC5, EXT1, FANCA, FANCD2, FANCI, FANCM, FBXW7, FGF10, FGF6, FGFR1, FGFR3, FLI1, FLT1, FLT3, GNAS, HNF1A, HRAS, KDR, KIT, KRAS, MAGI1, MAP2K1, MAP2K2, MAX, MED12, MET, MLH1, MMAB, MSH3, MSH6, MTOR, NF1, NFE2L2, NOTCH1, NOTCH2, NOTCH3, NRAS, NRG1, NTRK1, NTRK3, PDGFRA, PDGFRB, PIK3CA, PIK3CG, PIK3R1, PMS2, PPARG, PROC, PTCH1, RAD54L, RAF1, RECQL4, RET, ROS1, SF3B1, SF3B2, SLX4, SMO, TERT promoter, TET2, TP53BP1, TSC1, TSC2, WRN, XPA, XPC, ZNF395, B2M, HLA-A, HLA-B, HLA-C, TAP1, TAP2, NLRC5, IFNGR1, INFGR2, JAK1, JAK2, TP53, PTEN, and ARID1A.

In some aspects, the one or more tumor-naïve polynucleotide probes comprises two or more probes configured to capture all coding exon sequences of a given gene. In some aspects, the one or more tumor-naïve polynucleotide probes comprises two or more probes configured to capture a genomic region of interest associated-with cancer.

In some aspects, the tumor-informed polynucleotide probes and/or the tumor-naïve polynucleotide probes comprise probes that comprise overlapping sequences.

In some aspects, the panel comprises at least 20 probes, at least 30 probes, at least 40 probes, at least 50 probes, at least 60 probes, at least 70 probes, at least 80 probes, at least 90 probes, at least 100 probes, at least 200 probes, at least 300 probes, at least 400 probes, or at least 500 probes.

In some aspects, the panel is configured to cover at least 100 kb, at least 300 kb, at least 300 kb, at least 400 kb, between 100-400 kb, between 200-400 kb, between 300-400 kb, between 100-500 kb, between 200-500 kb, between 300-500 kb, or between 340-400 kb of the subject's genome.

In some aspects, the one or more tumor-naïve polynucleotide probes comprises polynucleotide probes configured to capture sequences associated with a given cancer the subject is known to have or suspected of having, optionally wherein the cancer is CRC or NSCLC.

In some aspects, the panel further comprises additional polynucleotide probes configured to capture sequences comprising polymorphisms in the human population, wherein the sequences comprising polymorphisms are capable in combination of uniquely identifying the subject.

Also provided herein is a method for enriching cfDNA, the method comprising: (a) providing a sample comprising cfDNA; (b) providing a panel of polynucleotide probes comprising any one of the tumor-informed/tumor-naïve combination panels provided herein; (c) contacting the sample comprising cfDNA with the panel of polynucleotide probes under conditions sufficient for cfDNA comprising a target sequence of interest to hybridize with its respective polynucleotide probe; and (d) capturing the hybridized cfDNA and polynucleotide probe pairs to enrich the cfDNA.

Also provided herein is a method for monitoring cancer status in a subject having, had, or suspected of having cancer, wherein the method comprises the steps of: a. obtaining or having obtained sequencing data of cell-free DNA (cfDNA) from a sample from the subject, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read depth is mean duplex read depth, and optionally wherein obtaining the sequencing data comprises collecting or having collected the sample from the subject, isolating or having isolated the cfDNA, enriching or having enriched the cfDNA, and/or sequencing or having sequenced the cfDNA; and b. determining or having determined a frequency of the mutations present in the exome to assess the status of the cancer, optionally wherein assessment of the status comprises assessment of presence and/or cancer burden, wherein the cfDNA has been enriched prior to sequencing using (1) a panel of subject-specific polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest.

Also provided herein is a method for monitoring cancer status in a subject having, had, or suspected of having cancer, wherein the method comprises the steps of: a. obtaining or having obtained sequencing data of cell-free DNA (cfDNA) from a sample from the subject, and wherein the sequencing data comprises a target coverage of at least 95% of all polynucleotide regions of interest corresponding to mutations present in an exome of the cancer, wherein the polynucleotide regions of interest comprise at least 50 mutations, and wherein the sequenced polynucleotide regions of interest comprise duplex read depth of at least 1000×, and optionally wherein obtaining the sequencing data comprises collecting or having collected the sample from the subject, isolating or having isolated the cfDNA, enriching or having enriched the cfDNA, and/or sequencing or having sequenced the cfDNA; and b. determining or having determined a frequency of the at least 50 mutations present in the exome to assess the status of the cancer, optionally wherein assessment of the status comprises assessment of presence and/or cancer burden, wherein the cfDNA has been enriched prior to sequencing using (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest.

Also provided herein is a method for assessing efficacy of a therapy in a subject, had, or suspected of having having cancer, wherein the method comprises the steps of: a. obtaining or having obtained sequencing data of cell-free DNA (cfDNA) from a pre-therapy sample from the subject, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read coverage is mean duplex read coverage, and optionally wherein obtaining the sequencing data comprises collecting or having collected the pre-therapy sample from the subject, isolating or having isolated the pre-therapy cfDNA, enriching or having enriched the pre-therapy cfDNA, and/or sequencing or having sequenced the pre-therapy cfDNA; b. obtaining or having obtained sequencing data of cell-free DNA (cfDNA) from a post-therapy sample from the subject, optionally wherein the therapy comprises a cancer vaccine comprising the neoantigen or expression system encoding the same, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read coverage is mean duplex read coverage, and optionally wherein obtaining the sequencing data comprises collecting or having collected the post-therapy sample from the subject, isolating or having isolated the post-therapy cfDNA, enriching or having enriched the post-therapy cfDNA, and/or sequencing or having sequenced the post-therapy cfDNA; and c. determining or having determined the frequency the mutations present in the exome of the pre-therapy cfDNA relative to the post-therapy cfDNA to assess the efficacy of the therapy, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable, wherein the cfDNA has been enriched prior to sequencing using (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest.

Also provided herein is a method for assessing efficacy of a therapy in a subject, had, or suspected of having having cancer, wherein the method comprises the steps of: a. obtaining or having obtained sequencing data of tumor-derived DNA from a cancer-diseased tissue from the subject, optionally wherein obtaining the sequencing data comprises collecting or having collected the cancer-diseased tissue, isolating or having isolated the tumor-derived DNA, and sequencing or having sequenced the tumor-derived DNA; b. determining or having determined one or more tumor-associated mutations relative to a wild-type germline nucleic acid sequence of the subject from the tumor-derived DNA sequencing data, optionally wherein one or more of the one or more tumor-associated mutations is associated with a neoantigen comprising at least one alteration that makes a peptide sequence encoded by the tumor-derived DNA distinct from the corresponding peptide sequence encoded by the wild-type germline nucleic acid sequence of the subject; c. designing and/or selecting or having designed and/or selected (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture at least the tumor-associated mutations optionally wherein the polynucleotide regions of interest comprise at least 50 tumor-associated mutations; d. obtaining or having obtained sequencing data of cell-free DNA (cfDNA) from a pre-therapy sample from the subject, wherein the pre-therapy cfDNA was enriched prior to sequencing using the polynucleotide probes, and wherein the sequencing data comprises a target coverage of at least 50% of all polynucleotide regions of interest corresponding to the tumor-associated mutations and wherein the sequenced polynucleotide regions of interest comprise read depth of at least 1000×, optionally wherein the mean read coverage is mean duplex read coverage, and optionally wherein obtaining the sequencing data comprises collecting or having collected the pre-therapy sample from the subject, isolating or having isolated the pre-therapy cfDNA, enriching or having enriched the pre-therapy cfDNA, and/or sequencing or having sequenced the pre-therapy cfDNA; e. obtaining or having obtained sequencing data of cell-free DNA (cfDNA) from a post-therapy sample from the subject, optionally wherein the therapy comprises a cancer vaccine comprising the neoantigen or expression system encoding the same, wherein the post-therapy cfDNA was enriched prior to sequencing using the polynucleotide probes, and wherein the sequencing data comprises a target coverage of at least 50% of all polynucleotide regions of interest corresponding to the tumor-associated mutations and wherein the sequenced polynucleotide regions of interest comprise read depth of at least 1000×, optionally wherein the mean read coverage is mean duplex read coverage, and optionally wherein obtaining the sequencing data comprises collecting or having collected the post-therapy sample from the subject, isolating or having isolated the post-therapy cfDNA, enriching or having enriched the post-therapy cfDNA, and/or sequencing or having sequenced the post-therapy cfDNA; and f. determining or having determined the frequency of the tumor-associated mutations of the pre-therapy cfDNA relative to the post-therapy cfDNA to assess the efficacy of the therapy, optionally wherein at least the one or more tumor-associated mutations associated with the neoantigen is determined, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable.

In some aspects, the method comprises designing and/or selecting or having designed and/or selected a combination panel of a panel of tumor-informed polynucleotide probes and a panel of tumor-naïve polynucleotide probes. In some aspects, the designed and/or selected combination panel comprises any one of the tumor-informed/tumor-naïve combination panels provided herein.

Also provided herein is a method for enriching cfDNA, the method comprising: (a) providing a sample comprising cfDNA; (b) providing a panel of polynucleotide probes, wherein the panel comprises: (i) one or more tumor-informed polynucleotide probes; and (ii) one or more tumor-naïve polynucleotide probes; (c) contacting the sample comprising cfDNA with the panel of polynucleotide probes under conditions sufficient for cfDNA comprising a target sequence of interest to hybridize with its respective polynucleotide probe; and (d) capturing the hybridized cfDNA and polynucleotide probe pairs to enrich the cfDNA. In some aspects, the panel comprises any one of the tumor-informed/tumor-naïve combination panels provided herein.

In some aspects, the method comprises one or more of the steps of: a. collecting or having collected the sample from the subject; b. isolating or having isolated the cfDNA; c. enriching or having enriched the cfDNA; or d. sequencing or having sequenced the cfDNA.

In some aspects, the method comprises each of the steps of: a. collecting or having collected the sample from the subject; b. isolating or having isolated the cfDNA; c. enriching or having enriched the cfDNA; and d. sequencing or having sequenced the cfDNA.

In some aspects, the mean read depth comprises at least 1500×, at least 2000×, at least 2500×, 3000×, at least 3500×, at least 4000×, at least 4500×, or at least 5000× mean read coverage. In some aspects, the mean read depth comprises a range from 1000× to 5000× mean read coverage. In some aspects, the mean read depth comprises a range from 1000× to 4000×, 1000× to 3000×, 1000× to 2000×, 2000× to 5000×, 2000× to 4000×, 2000× to 3000×, 3000× to 5000×, 3000× to 4000×, or 4000× to 5000× mean read coverage. In some aspects, the mean read depth comprises mean read duplex depth.

In some aspects, each of the polynucleotide regions of interest corresponding to the mutations present in the exome comprise a read depth of at least 1000×. In some aspects, each of the polynucleotide regions of interest corresponding to the mutations present in the exome comprise a read depth of at least 1000×, at least 1500×, at least 2000×, at least 2500×, 3000×, at least 3500×, at least 4000×, at least 4500×, or at least 5000×. In some aspects, the target coverage comprises at least 60%, at least 70%, at least 80%, or at least 90% of polynucleotide regions of interest corresponding to the mutations present in the exome of the cancer. In some aspects, the target coverage comprises at least 95%, at least 96%, at least 97%, at least 98%, at least 99%, at least 99.5%, at least 99.9%, or 100% of polynucleotide regions of interest corresponding to the mutations present in the exome of the cancer.

In some aspects, the target coverage comprises at least 95% of polynucleotide regions of interest corresponding to the mutations present in the exome of the cancer. In some aspects, the polynucleotide regions of interest comprise at least 50, at least 60, at least 70, at least 80, or at least 90 mutations.

In some aspects, the polynucleotide regions of interest comprise at least 50 mutations. In some aspects, the polynucleotide regions of interest comprise at least 100, at least 150, at least 200, at least 250, at least 300, at least 400, at least 500, at least 600, at least 700, at least 800, at least 900, or at least 1000 mutations.

In some aspects, the method comprises the steps of: a. obtaining or having obtained sequencing data of tumor-derived DNA from a cancer-diseased tissue from the subject, optionally wherein obtaining the sequencing data comprises collecting or having collected the cancer-diseased tissue, isolating or having isolated the tumor-derived DNA, and sequencing or having sequenced the tumor-derived DNA; b. determining or having determined one or more tumor-associated mutations relative to a wild-type germline nucleic acid sequence of the subject from the tumor-derived DNA sequencing data, optionally wherein one or more of the one or more tumor-associated mutations is associated with a neoantigen comprising at least one alteration that makes a peptide sequence encoded by the tumor-derived DNA distinct from the corresponding peptide sequence encoded by the wild-type germline nucleic acid sequence of the subject; c. designing and/or selecting or having designed and/or selected (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest corresponding to the tumor-associated mutations optionally wherein the polynucleotide regions of interest comprise at least 50 tumor-associated mutations; and d. enriching or having enriched the cfDNA using the polynucleotide probes prior to sequencing.

In some aspects, the cancer is selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer.

In some aspects, the subject has been administered a therapy. In some aspects, the therapy comprises a cancer vaccine. In some aspects, the cancer vaccine comprises an epitope-encoding nucleic acid sequence encoding at least one of the mutations present in the exome of the cancer. In some aspects, the cancer vaccine comprises a self-amplifying alphavirus-based expression system. In some aspects, the cancer vaccine comprises a chimpanzee adenovirus (ChAdV)-based expression system.

In some aspects, the method comprises obtaining sequencing data of cfDNA from two or more samples from the subject. In some aspects, the two or more samples are collected at different time points. In some aspects, the two or more samples are collected at different time points relative to administration of a therapy. In some aspects, a pre-therapy sample is collected prior to administration of the therapy and a post-therapy cfDNA is collected subsequent to administration of the therapy. In some aspects, the determining step comprises determining or having determined the frequency of the mutations of the pre-therapy cfDNA relative to the post-therapy cfDNA to assess the efficacy of the therapy, optionally wherein at least the one or more tumor-associated mutations associated with the neoantigen is determined, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable.

In some aspects, an increase in the frequency of one or more of the mutations in the tumor-naïve panel in the post-therapy cfDNA relative to the pre-therapy cfDNA indicates a likelihood of an immune evasion mechanism tumor mutation. In some aspects, an increase in the frequency of the mutations in the post-therapy cfDNA relative to the pre-therapy cfDNA indicates an increased likelihood that tumor burden of the subject is increasing. In some aspects, a decrease or maintenance of the frequency of the mutations in the post-therapy cfDNA relative to the pre-therapy cfDNA indicates an increased likelihood that tumor burden of the subject is decreasing or stable. In some aspects, the decrease comprises a Complete Response (CR) or a Partial Response (PR).

In some aspects, the method further comprises administering a therapy to the subject following the assessment of the status of the cancer. In some aspects, the assessment of the frequency of the mutations in the cfDNA indicates a likelihood the subject has or still has cancer.

In some aspects, the therapy comprises a cancer vaccine. In some aspects, the cancer vaccine comprises an epitope-encoding nucleic acid sequence encoding at least one of the mutations present in the exome. In some aspects, the cancer vaccine comprises a self-amplifying alphavirus-based expression system. In some aspects, the cancer vaccine comprises a chimpanzee adenovirus (ChAdV)-based expression system.

In some aspects, the collecting step comprises collecting a blood sample.

In some aspects, the isolation step comprises centrifugation to separate cfDNA from cells and/or cellular debris. In some aspects, the isolation step comprises isolating cfDNA from whole blood. In some aspects, isolating cfDNA from whole blood comprises separating the plasma layer, buffy coat, and red blood cells. In some aspects, the cfDNA is isolated from the plasma layer.

In some aspects, the sequencing step comprises next generation sequencing (NGS) or Sanger sequencing. In some aspects, NGS comprises duplex sequencing, whole-exome sequencing, whole-genome sequencing, de novo sequencing, phased sequencing, targeted amplicon sequencing, or shotgun sequencing.

In some aspects, the enrichment step comprises enriching the cfDNA for the polynucleotide regions of interest corresponding to the mutations present in the exome prior to sequencing. In some aspects, the enrichment comprises the combination of the panel of tumor-informed polynucleotide probes and the panel of tumor-naïve polynucleotide probes. In some aspects, separate samples are separately enriched for each of the panel of tumor-informed polynucleotide probes and the panel of tumor-naïve polynucleotide probes.

In some aspects, the tumor-informed polynucleotide probes comprises each of the polynucleotide regions of interest corresponding to the mutations present in the exome. In some aspects, the tumor-informed polynucleotide probes comprises at least 50%, at least 60%, at least 70%, at least 80%, at least 90%, at least 95%, at least 96%, at least 97%, at least 98%, at least 99%, at least 99.5%, at least 99.9%, or 100% of polynucleotide regions of interest corresponding to the mutations present in the exome of the cancer. In some aspects, the tumor-informed polynucleotide probes comprises at least 50, at least 60, at least 70, at least 80, at least 90 mutations, at least 100, at least 150, at least 200, at least 250, at least 300, at least 400, at least 500, at least 600, at least 700, at least 800, at least 900, or at least 1000 mutations, optionally the mutations present in the exome of the cancer.

In some aspects, the enrichment step comprises hybridizing one or more polynucleotide probes to the one or more polynucleotide regions of interest.

In some aspects, the polynucleotide probes are 80 to 150 base pairs (bp) in length. In some aspects, the polynucleotide probes are 50-100, 50-150, 80 to 140, 80 to 130, 80 to 120, 80 to 110, 80 to 100, 80 to 90, 90 to 150, 90 to 140, 90 to 130, 90 to 120, 90 to 110, 90 to 100, 100 to 150,100 to 140, 100 to 130, 100 to 120,100 to 110, 110 to 150, 110 to 140, 110 to 130, 110 to 120, 120 to 150, 120 to 140, 120 to 130,130 to 150, 130 to 140, 140 to 150, 50, 60, 70, 80, 90, 100, 110, 120, 130, 140, or 150 bp in length.

In some aspects, the one or more polynucleotide probes are biotinylated.

In some aspects, the tumor-informed polynucleotide probes are designed or selected following sequencing of a tumor of the subject. In some aspects, the tumor-informed polynucleotide probes are designed or selected following exome sequencing of the tumor of the subject. In some aspects, the tumor-informed polynucleotide probes are designed or selected to target all mutations of the sequenced tumor.

In some aspects, the sequencing step comprises ligating sequencing adaptors to the cfDNA. In some aspects, the sequencing adaptors are configured for duplex sequencing.

In some aspects, one or more of the mutations comprises a point mutation, a frameshift mutation, a non-frameshift mutation, a deletion mutation, an insertion mutation, a splice variant, a genomic rearrangement, a proteasome-generated spliced antigen, or combinations thereof. In some aspects, one or more of the mutations comprises at least one alteration that makes a peptide sequence encoded by the cfDNA distinct from the corresponding peptide sequence encoded by the wild-type germline nucleic acid sequence of the subject. In some aspects, the one or more mutations consists of coding mutations comprising at least one alteration that makes a peptide sequence encoded by the cfDNA distinct from the corresponding peptide sequence encoded by the wild-type germline nucleic acid sequence of the subject.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows a detailed pipeline for the isolation and processing of ctDNA from patient. Briefly, tumor-specific DNA variant alleles are identified from biopsied tumor tissue (point 1). Blood is drawn from patients at specific points of their dosing schedules, and ctDNA is isolated and used to generate a UMI library (points 2 and 4). Baits designed based on variants identified in patient tumor DNA (point 3) are used to purify ctDNA containing identified variants (point 5).

FIG. 2 shows a detailed pipeline following the isolation and processing of ctDNA for analysis from patient following isolation and processing as outlined in FIG. 1. Purified ctDNA is sequenced (point 6) to quantify prevalence of specific identified variants. Repeated testing of ctDNA over the course of treatment allows monitoring of tumor progression or response to therapy.

FIG. 3A exemplifies the isolation and sequencing of circulating tumor DNA (ctDNA) in two patients receiving GRANITE therapy and is a graph showing the absolute duplex read coverage of identified DNA variants in ctDNA isolated from Patient #1 (identified as pt0009).

FIG. 3B exemplifies the isolation and sequencing of circulating tumor DNA (ctDNA) in two patients receiving GRANITE therapy and is a graph showing the normalized duplex read coverage of identified DNA variants in ctDNA isolated from Patient #1 (identified as pt0009).

FIG. 3C exemplifies the isolation and sequencing of circulating tumor DNA (ctDNA) in two patients receiving GRANITE therapy and is a graph showing the monitoring of tumor-specific DNA variant alleles in Patient #1 over the course of treatment, with TP52 R175H, APC T1556fs, and CDKN2A W110* highlighted.

FIG. 3D exemplifies the isolation and sequencing of circulating tumor DNA (ctDNA) in two patients receiving GRANITE therapy and is a graph showing the absolute duplex read coverage of identified DNA variants in ctDNA isolated from Patient #2 (identified as pt0005).

FIG. 3E exemplifies the isolation and sequencing of circulating tumor DNA (ctDNA) in two patients receiving GRANITE therapy and is a graph showing the normalized duplex read coverage of identified DNA variants in ctDNA isolated from Patient #2 (identified as pt0005).

FIG. 3F exemplifies the isolation and sequencing of circulating tumor DNA (ctDNA) in two patients receiving GRANITE therapy and is a graph showing the monitoring of tumor-specific DNA variant alleles in Patient #2 over the course of treatment, including TRABD2B A385T, ADAR G751R, VILL L273fs, SURF2 P146L, TP53 P153fs, CSH2 A156V, and MAP2K2 E66K.

FIG. 4A is a graph showing the monitoring of variant allele frequency (VAF) in Patient #1 (pt0009) over the course of GRANITE therapy and shows the frequency of 11 identified tumor-specific variant alleles over the course of treatment.

FIG. 4B is a graph showing the monitoring of variant allele frequency (VAF) in Patient #1 (pt0009) over the course of GRANITE therapy and shows the trend in VAF of all variant alleles in isolated ctDNA over the course of treatment.

FIG. 4C is a graph showing the monitoring of variant allele frequency (VAF) in Patient #1 (pt0009) over the course of GRANITE therapy and shows the average percent change in VAF between consecutive dosages over the course of treatment.

FIG. 5A is a graph that exemplifies the monitoring of ctDNA in additional patients receiving GRANITE therapy and shows the monitoring of ctDNA in a patient with non-small cell lung cancer (NSCLC) who received GRANITE therapy.

FIG. 5B is a graph that exemplifies the monitoring of ctDNA in additional patients receiving GRANITE therapy and shows the tracking of ctDNA in a patient with microsatellite-stable colorectal cancer (MSS-CRC).

FIG. 6A is a graph that shows the monitoring of ctDNA in a patient receiving SLATE therapy (identified as pt0101) and shows the absolute duplex read coverage of specified KRAS allele variants in ctDNA isolated from patient plasma.

FIG. 6B is a graph that shows the monitoring of ctDNA in a patient receiving SLATE therapy (identified as pt0101) and shows the normalized duplex read coverage of specified KRAS allele variants in ctDNA isolated from patient plasma.

FIG. 6C is a graph that shows the monitoring of ctDNA in a patient receiving SLATE therapy (identified as pt0101) and shows the changes in KRAS variant allele duplexes between consecutive doses.

FIG. 7 is a graph that shows the monitoring of ctDNA associated with the KRAS G12C mutation in a patient with NSCLC.

FIG. 8 shows a detailed pipeline for the isolation and processing of ctDNA from patients for patient-specific vaccine screening and manufacture.

FIG. 9 shows a schematic for the ctDNA monitoring assay. Shotgun libraries from cfDNA, biopsy DNA, or gDNA from whole blood were prepared with duplex UMIs. Duplex sequencing reduces noise by requiring variants to be observed on both strands of a duplex molecule. Multiple patient-specific sets were combined to create a superset containing probes for 6-9 patients. The universal panel captured a set of common targets in all patient samples.

FIG. 10A shows the number of potential variants covered by the indicated NGS panels. FIG. 10B shows the percentage of WES variants potentially covered by various NGS panels.

FIG. 11 shows the blood collection protocol for patients enrolled in SLATE (“off-the-shelf” vaccine program) and GRANITE (“personalized cancer vaccine” program).

FIG. 12 shows that patient assays monitored an average of approximately 140 variants per patient at high sequencing depth for variant calling at >1000× duplex consensus coverage.

FIG. 13A shows the cassette mutations observed in the indicated patient's ctDNA and biopsies. * indicate patients with unavailable biopsies or where the tumor content was too low to detect variants in the assay. FIG. 13B shows that significant overlap was found when comparing the variants in cfDNA and corresponding biopsies using the GRANITE assay, especially with the ability to call variants at a lower frequency in high-quality (RNALater or fresh frozen) biopsies.

FIG. 14A shows the presence of de novo variants in the cfDNA samples from the indicated patient and tumor tissue type (GEA, CRC, or NSCLC). Many patients had additional variants present in their cfDNA that were not in the original biopsy. The new variants often occurred where another patient had a targeted variant. FIG. 14B shows that CHIP mutations were identified and ruled out as somatic tumor variants using the matched normal gDNA from whole blood or PMBCs. FIG. 14C shows a representative patient G08 with two NLRC5 mutations, one of which tracked with the average VAF of all variants, and two TAP1 mutations that appeared after nearly a year on therapy. FIG. 14D shows an additional analysis summary of variants observed in cfDNA that were found outside of the patient-specific variants. FIG. 14E shows G09 cfDNA dynamics of new variants including multiple KRAS variants.

FIG. 15 shows that all patient-specific variants captured in WES of the biopsy were also captured using the patient-specific assay (100% concordance).

FIG. 16A shows that the variants in the baseline biopsy were at a low frequency in the archival biopsy. FIG. 16B shows that the on-treatment biopsy variants were more representative of those present in the archival biopsy. Despite all biopsies being from the primary site, only 12/135 of the targeted variants were shared among the three, indicating tumor heterogeneity.

FIG. 17A shows the variant dynamics in cfDNA over time in patient G01. FIG. 17B shows the targeted low frequency variants in ctDNA for the indicated variants (SSH3, GRIA4, ZNF541, TMEM217, ZNF697, AHNAK2, SCHIP1, and CNR1). FIG. 17C shows the targeted variants in the WES of ctDNA of patient G01 over time. FIG. 17D shows the brain met biopsy variants in the WES of ctDNA of patient G01 over time.

FIG. 18A-E show ctDNA monitoring of tumor variants in SLATE patients. FIG. 18A shows the ctDNA % VAF for patient S2. FIG. 18B shows the ctDNA % VAF for patient S5. FIG. 18C shows the ctDNA % VAF for patient S10. FIG. 18D shows the ctDNA % VAF for patient S13. FIG. 18E provides a representative patient with no MR, showing loss of B2M start codon. SD=stable disease, PD=progressive disease, best overall response is denoted.

FIG. 19A shows the fold change in the HLA allele read fraction from the molecular responder (MR). FIG. 19B shows the fold change in the HLA allele read fraction from the non-molecular responder (non-MR).

FIG. 20 provides a diagram outlining the considerations for inclusion of subject-specific, tumor-informed probes.

FIG. 21A provides the percentage of CRC and NSCLC samples that are covered by the target probes in the Universal Panel. The data is based on analysis of 10,586 samples from cbioportal.org. FIG. 21B shows a retrospective analysis of the variants in prior study (GO-004) patients identified by the universal panels version 1 (v1) or version 2 (v2).

FIG. 22 shows the general strategy for monitoring chromosome 6 for loss-of-heterozygosity for HLA genes. SEQ ID NOs: 1-6 are shown respectively, top to bottom, for the six sequences displayed.

### DETAILED DESCRIPTION

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. Such shared neoantigens are useful for inducing an immune response in a subject via administration. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISpot” means Enzyme-linked immunosorbent spot assay—which is a common method for monitoring immune responses in humans and animals.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, and smoking history.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a 26S promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, or other 26S subgenomic promoter sequence, a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between.

The term “pharmaceutically effective amount” is an amount of a vaccine component (such as a peptide, engineered vector, and/or adjuvant) that is effective in a route of administration to provide a cell with sufficient levels of protein, protein expression, and/or cell-signaling activity (e.g., adjuvant-mediated activation) to provide a vaccinal benefit, i.e., some measurable level of immunity.

Terms such as “obtaining,” “isolating,” “enriching,” “sequencing,” “acquiring,” “collecting,” and “determining” as used herein refers to directly performing a process (e.g., directly performing a method) to acquire a result, such as directly acquiring a product, including, but not limited to, directly sequencing cfDNA to acquire cfDNA sequencing data, directly isolating cfDNA to acquire isolated cfDNA, directly enriching cfDNA to acquire enriched cfDNA samples including cfDNA, etc. Terms such as “having obtained,” “having isolated,” “having enriched,” “having sequenced,” “having acquired,” “having collected,” and “having determined” as used herein refers to indirectly receiving information or receiving a product without directly performing a process (e.g., without directly performing a method), such as by receiving the knowledge or product from another party or source (e.g., from a third party laboratory that itself directly acquired the cfDNA sequencing data, isolated cfDNA, enriched cfDNA, and/or collect a sample including cfDNA, etc.). In some instances, the other party or source is directed to directly perform a process (e.g., a third party laboratory directed to acquire cfDNA sequencing data, isolate cfDNA, enrich cfDNA, and/or collect a sample including cfDNA, etc.). In some instances, the knowledge or product is purchased from another party or source that directly performed a process (e.g., purchasing cfDNA sequencing data, isolated cfDNA, enriched cfDNA, and/or a collected sample including cfDNA, etc.).

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### Monitoring Disease Status and Therapy Efficacy

Provided herein are methods for monitoring disease status in a subject through analysis of cell-free DNA (cfDNA), particularly through monitoring mutation frequency (e.g., tumor associated mutations associated with a cancer). For example, cfDNA can be used to monitor the progression of disease in patients receiving therapy. The methods of cfDNA analysis described herein provide a non-invasive manner of assessing and/or monitoring disease, in particular relative to the more invasive procedures such as tumor biopsies. The methods of cfDNA analysis described herein are particularly useful for analyzing large numbers of mutations, such as analyzing all or the majority of a tumor's exome. In general, the monitoring is performed through sequencing of cfDNA with both broad target coverage (e.g., at least 50% of all polynucleotide regions of interest corresponding to mutations present in a cancer exome of a subject) and a high read depth of sequencing (“deep sequenced,” e.g., a mean read depth of at least 1000×).

In one aspect, methods for monitoring cancer status in a subject includes the steps of: a. obtaining or having obtained sequencing data of cfDNA from a sample from a subject, and wherein the sequencing data includes a target coverage of at least 50% of all polynucleotide regions of interest corresponding to mutations present in an exome of the cancer and wherein the sequenced polynucleotide regions of interest have a mean read depth of at least 1000×; and b. determining or having determined a frequency of the mutations present in the exome to assess the status of the cancer.

More than one sample can be analyzed to assess the status of a disease in the subject. Accordingly, in one aspect, methods for monitoring cancer status in a subject includes the steps of: a. obtaining or having obtained sequencing data of cfDNA from a first sample from the subject, and wherein the sequencing data includes a target coverage of at least 50% of all polynucleotide regions of interest corresponding to mutations present in an exome of the cancer and wherein the sequenced polynucleotide regions of interest have a mean read depth of at least 1000×; b. obtaining or having obtained sequencing data of cfDNA from a second sample from the subject wherein the sequencing data includes a target coverage of at least 50% of all polynucleotide regions of interest corresponding to mutations present in an exome of the cancer and wherein the sequenced polynucleotide regions of interest have a mean read depth of at least 1000×; and c. determining or having determined the frequency the mutations present in the exome of the first cfDNA relative to the second cfDNA to assess the status of the cancer.

Multiple samples containing cfDNA can be collected from a subject at different time points and used to monitor a disease, such as monitoring disease burden and/or response to a therapy over the course of treatment. Time points can be selected to monitor disease status as specific intervals. For example, time points can be selected based on therapy dosing schedule. Time points based on dosing schedules can include the same day as administration of a therapy. Time points based on dosing schedules can include, but are not limited to, one day, two days, three days, four days, five days, six days after a dose. Time points based on dosing schedules can include, but are not limited to, one week, two weeks, three weeks, four weeks, five weeks, six weeks, eight weeks, ten weeks, twelve weeks after a dose. Time points based on dosing schedules can include, but are not limited to, one month, two months, three months, six months, and twelve months after a dose.

Time points can be at regular time intervals, such as regular time intervals over the course of therapy, including, but not limited to, every day, every two days, every three days, every four days, every five days, every six days. Time points based on regular time intervals can include, but are not limited to, once every week, once every two weeks, once every three weeks, once every four weeks, once every five weeks, once every six weeks, every eight weeks, every ten weeks, every twelve weeks. Time points can also be selected base on regular time intervals including, but not limited to, once every month, once every two months, once every three months, once every six months, and once every twelve months. Combinations of one or more of the above mentioned time intervals may also be used.

Analysis of cfDNA can be used to monitor the progression of disease in patients receiving a therapy. For example, longitudinal samples can be collected over the course of therapy to monitor cancer status (e.g., tumor burden over time). Increases in the frequency of monitored mutations over longitudinal samples can indicate an increased likelihood that tumor burden of the subject is increasing. Decreases or maintenance of the frequency of the mutations in of monitored mutations over longitudinal samples can indicate an increased likelihood that tumor burden of the subject is decreasing or stable.

Analysis of cfDNA can be used to asses efficacy of a therapy administered to a subject. Accordingly, in one aspect, methods for assessing efficacy of a therapy in a subject having cancer includes the steps of: a. obtaining or having obtained sequencing data of cfDNA from a pre-therapy sample from the subject, and wherein the sequencing data includes a target coverage of at least 50% of all polynucleotide regions of interest corresponding to mutations present in an exome of the cancer and wherein the sequenced polynucleotide regions of interest have a mean read depth of at least 1000×; b. obtaining or having obtained sequencing data of cfDNA from a post-therapy sample from the subject, wherein the sequencing data includes a target coverage of at least 50% of all polynucleotide regions of interest corresponding to mutations present in an exome of the cancer and wherein the sequenced polynucleotide regions of interest have a mean read depth of at least 1000×; and c. determining or having determined the frequency the mutations present in the exome of the pre-therapy cfDNA relative to the post-therapy cfDNA to assess the efficacy of the therapy.

Multiple samples having cfDNA can be collected at different time points relative to administration of a therapy. Samples having cfDNA can be collected prior to administration of a therapy. Samples having cfDNA can be collected subsequent to administration of a therapy. Samples having cfDNA can be collected concurrently with administration of a therapy. Samples having cfDNA can be collected both prior to and subsequent to administration of a therapy. For example, a first sample having cfDNA can be collected prior to administration of a therapy to a subject and a second sample having cfDNA can be collected subsequent to administration of the therapy. Samples having cfDNA can be collected both concurrently with and subsequent to administration of a therapy. For example, a first sample having cfDNA can be collected concurrently with administration of a therapy to a subject and a second sample having cfDNA can be collected subsequent to administration of the therapy. Multiple samples (e.g., longitudinal samples) having cfDNA can be collected subsequent to administration of a therapy.

Obtaining the sequencing data can include one or more of the following steps: collecting or having collected a sample from a subject; isolating or having isolated cfDNA; enriching or having enriched cfDNA, and/or sequencing or having sequenced cfDNA. Obtaining the sequencing data can include each of the following steps: collecting or having collected a sample from a subject; isolating or having isolated cfDNA; enriching or having enriched cfDNA, and/or sequencing or having sequenced cfDNA. An intermediate can be acquired for performing any of the above steps. For example, isolated cfDNA can be acquired from a third-party source and used for performing one or more of the remaining steps, such as enrichment and sequencing. An intermediate can be produced and a third-party directed to perform any of the above steps. For example, enriched cfDNA can be produced and provided to a third-party source for performing one or more of the remaining steps, such as sequencing.

### Cancer Monitoring

Methods described herein can be used to monitor cancer status, such as tumor burden.

A subject's disease can include cancer. Cancer cells can release their genomic DNA into the circulation upon cell death, referred to as circulating tumor DNA (ctDNA) or as cfDNA from a cancer cell. A variety of cancers can be monitored. For example, cancers that can be monitored include but are not limited to, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, Hurthle cell adenoma, renal cell carcinoma, Grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, Warthin's tumor, thymoma, specialized gonadal neoplasms, sex cord stromal tumor, thecoma, granulosa cell tumor, arrhenoblastoma, Sertoli-Leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as Waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin's lymphomas (nodular sclerosis, mixed cellularity, lymphocyte-rich, lymphocyte depleted or not depleted), and nodular lymphocyte-predominant Hodgkin's lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. Cancers that can be monitored include but are not limited to lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer.

Cancer monitoring can also include monitoring for cancer evasion mutations (also referred to as secondary mutations or escape mutants). For example, cancer monitoring can include monitoring for de novo mutations relative to an earlier sequencing dataset, such as an initial biopsy, longitudinal sample, pre-therapy sample, or any other archival sample. Monitoring for cancer evasion mutations can inform whether additional therapy is warranted (e.g., a therapy effective against cancers with the particular de novo mutation) and/or whether efficacy of the current or proposed therapy will be impacted. Cancer evasion mutations include those genes targeted by tumor-naïve probe panels described herein, such as genes and mutations generally considered oncogenic (e.g., “driver” mutations thought to promote cancer and generally considered gain-of-function mutations), tumor-suppressor genes (e.g., genes generally considered to monitor and/or control tumor-associated properties, such as cell division, where mutations can interfere with controlling such properties and are generally considered loss-of-function mutations), interferon-y signaling pathway genes (including JAK/STAT signaling pathway genes), antigen-processing pathway genes (including monitoring for HLA loss of heterozygosity), and mutations otherwise generally associated with cancer but may not be otherwise annotated (e.g., not yet annotated as an oncogene or tumor-suppressor).

The tumor-informed/tumor-naïve combination panels described herein can simultaneously monitor for cancer status, such as tumor burden, as well as cancer evasion mutations using a single panel.

### Tumor Specific Mutations

Methods described herein are applicable to the tracking of the presence of tumor specific mutations associated with cancer cells that are present in cfDNA (“ctDNA”). Tumor specific mutations can include previously identified tumor specific mutations, for example found at the Catalogue of Somatic Mutations in Cancer (COSMIC) database.

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in international patent application publications WO/2017/106638, WO/2018/195357, and WO/2018/208856, each of which are herein incorporated by reference, in their entirety, for all purposes.

Genetic mutations in tumors can be considered useful for the immunological targeting of tumors and/or monitoring tumor burden (e.g., disease status) if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of non-frameshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA, or protein in tumor versus normal cells.

A variety of methods are available for detecting the presence of a particular mutation or allele in an individual's DNA or RNA. Any of the sequencing methods described herein can be used to determine tumor specific mutations. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. Capture primers function as a fixed position site for sequence determination using multiple cycles of synthesis, detection, and chemical cleavage of the dye-linker to remove the dye. Each cycle adds the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to isolate nucleic acid samples for use in methods of identifying tumor specific mutations described herein. For example, a DNA or RNA sample can be isolated from a tumor or a bodily fluid, e.g., blood, collected by known techniques (e.g. venipuncture) or saliva. Alternatively, nucleic acid tests can be performed on dry samples (e.g. hair or skin). In addition, a sample can be collected for sequencing from a tumor and another sample can be collected from normal tissue for sequencing where the normal tissue is of the same tissue type as the tumor. A sample can be collected for sequencing from a tumor and another sample can be obtained from normal tissue for sequencing where the normal tissue is of a distinct tissue type relative to the tumor. Tumors from which tumor specific mutations can be identified include, but are not limited to, any of the tumors described herein, such as lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer. Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

Processing of cfDNA

Methods for processing cfDNA (e.g., isolation and purification of cfDNA) are generally known to those skilled in the art. For example, general methods for isolating cfDNA are described in US-2020/0277667-A1, which is herein incorporated by reference for all purposes. See also, e.g., Current Protocols in Molecular Biology, latest edition. Exemplary methods for isolating cfDNA are also described in U.S. Pat. No. 10,385,369-B2 and US-2020/0277667-A1, Cell-Free Plasma DNA as a Predictor of Outcome in Severe Sepsis and Septic Shock. Clin. Chem. 2008, v. 54, p. 1000—Diagnostics. Clin. Chem 1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular. 2003, v. 49, p. 1953-1955; Chiu R W K, Poon L M, Lau T K, Leung T N, Wong E M C, Lo Y M D. effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526, each of which is herein incorporated by reference for all purposes.

Commercially available kits for isolation and purification of cfDNA are known to those skilled in the art including, but not limited to, the QIAamp circulating nucleic acid kit and the Apostle MiniMax cfDNA Isolation Kit (Beckman Coulter; Indianapolis, IN).

Blood/plasma samples can be collected from a subject and cfDNA can be isolated from the blood/plasma samples. Samples having cfDNA other than blood can be collected (e.g., stool, mucus) for cfDNA isolation and purification. Isolation of cfDNA can occur, for example, through centrifugation to separate cfDNA from cells or cellular debris or from whole blood by separation of the plasma layer, which can contain cfDNA, from the buffy coat and red blood cells. Whole blood can be collected in cell-free DNA BCT tubes, centrifuged at an appropriate speed to separate the plasma layer, buffy coat, and red bloods. The plasma layer can then be removed and spun again to remove any residual cellular material. The supernatant can then be collected and stored at −80° C. until extraction. As an exemplary, non-limiting example, whole blood can be collected in 10 mL Streck cell-free DNA BCT tubes (Streck; La Vista, NE, USA), spun at 1600×g for 10 minutes at ambient temperature to separate the plasma layer, buffy coat, and red bloods. The plasma layer can then be removed and spun again at 5000×g for 10 minutes to remove any residual cellular material. The supernatant can then collected and stored at −80° C. until extraction. One having ordinary skill in the art can recognize that the above non-limiting exemplary protocol can be optimized based on specific experimental conditions.

To prepare a cfDNA library for sequencing, the cfDNA is generally fragmented, for example, sheared or enzymatically prepared (e.g., fragmented using a NEBNext Ultra II FS DNA Module; NEB, Ipswich, MA), to produce a library of polynucleotide regions of interest. Isolated nucleic acid (e.g., isolated cfDNA) can be fragmented or sheared by practicing routine techniques. For example, DNA can be fragmented by physical shearing methods, enzymatic cleavage methods, chemical cleavage methods, and other methods well known to those skilled in the art. One having ordinary skill in the art can recognize that the above non-limiting illustrative protocols can be optimized for producing a library of desired fragment length depending on desired sequencing applications, such as optimized for exome sequencing. For example, the time of enzymatic digestion can be optimized (e.g., as an illustrative example, 25 minutes using a NEBNext Ultra II FS DNA Module). Fragment length can be at least 100, at least 150, at least 200, at least 250, at least 300, at least 400, at least 500, at least 600, at least 700, at least 800, at least 900, or at least 1000 bp in length. Fragment length can be 100-250, 150-350, 200-450, 300-700, or 500-1000 bp in length. Fragment length can average at least 100, at least 150, at least 200, at least 250, at least 300, at least 400, at least 500, at least 600, at least 700, at least 800, at least 900, or at least 1000 bp in length. Fragment length can average 100-250, 150-350, 200-450, 300-700, or 500-1000 bp in length.

cfDNA Enrichment

cfDNA can be enriched to improve detection and measurement of specific polynucleotide regions of interest. Typically, enrichment is performed on a library of fragmented cfDNA (e.g., a library of polynucleotide regions of interest). Regions of interest can comprise polynucleotides known or suspected to encode one or more mutations. Regions of interest can also comprise gene translocations (e.g., Bcr-Abl fusion). Regions of interest can comprise polynucleotides encoding a gene coding region or a fragment of a gene coding region, which can include tumor exome polynucleotides, such as tumor exome polynucleotides known or suspected of having subject and/or tumor specific mutations. Enrichment of polynucleotide regions of interest in general can improve targeted measurement of DNA regions of interest (e.g., increasing sensitivity) through subtracting noise from sequencing results. The terms “enrich” and “enrichment” refers to a partial purification of analytes that have a certain feature (e.g., nucleic acids that are known or suspected to have tumor-specific mutations) from analytes that do not have the feature (e.g., nucleic acids that do not contain tumor-specific mutations). Enrichment typically increases the concentration of the analytes that have the feature (e.g., nucleic acids that contain tumor-specific mutations) by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more tumor-specific mutations that have been modified to contain a capture tag.

Enriching cfDNA can comprise hybridizing one or more polynucleotide probes (also referred to herein as “baits”) to the one or more polynucleotide regions of interest. Bait sequences can be based on tumor-specific mutations derived from genomic sequencing, such as sequencing of a tumor exome of a biopsy. Baits can comprise a single polynucleotide sequence or a library of polynucleotide sequences derived from tumor sequencing. Bait sequences derived from tumor sequencing can be subject-specific. For example, a subject's tumor can be biopsied and sequenced to determine mutations associated with the subject's tumor, following which the subject and tumor-specific sequences can be used to design subject-specific baits for enriching regions of interest of the tumor exome, including baits capable of enriching all regions of interest having patient specific-tumor variants.

Baits can include panels that include a combination of tumor-informed polynucleotide probes and tumor-naïve polynucleotide probes (also referred to as “combination panels”).

Tumor-informed polynucleotide probes include probes that are configured to capture a target sequence (e.g., through hybridization and other modifications, such as biotinylation, as described elsewhere herein). Target sequences can include an epitope sequence encoded by a cancer vaccine administered to a subject, wherein the subject has been determined to have a tumor expressing the epitope sequence. For example, probes to such epitope sequences can be considered tumor-informed when given a cancer vaccine administered to a subject when either (a) the vaccine is a personalized vaccine, such that prior cancer/tumor sequencing informs the selection of epitopes for inclusion in the cancer vaccine itself, or (b) the vaccine is an “off-the-shelf” vaccine, where the vaccine includes commonly occurring epitopes but requires prior cancer/tumor sequencing to determine if a subject meets the eligibility requirements to received the vaccine.

Exemplary epitope sequences that can be encoded by a cancer vaccine include epitopes have a mutation including, but not limited to, KRAS, G13D, KRAS_Q61K, TP53_R249M, CTNNB1_S45P, CTNNB1_S45F, ERBB2_Y772_A775dup, KRAS_G12D, KRAS_Q61R, CTNNB1_T41A, TP53_K132N, KRAS_G12A, KRAS_Q61L, TP53_R213L, BRAF_G466V, KRAS_G12V, KRAS_Q61H, CTNNB1_S37F, TP53_S127Y, TP53_K132E, and KRAS_G12C.

Exemplary epitope sequences that can be encoded by a cancer vaccine include a KRAS mutation, such as a KRAS_G12C mutation, a KRAS_G12D mutation, a KRAS_G12V mutation, and a KRAS_Q61H mutation. Exemplary epitope sequences that can be encoded by a cancer vaccine include a KRAS_G12C mutation. Exemplary epitope sequences that can be encoded by a cancer vaccine include a KRAS_G12D mutation. Exemplary epitope sequences that can be encoded by a cancer vaccine include a KRAS_G12V mutation. Exemplary epitope sequences that can be encoded by a cancer vaccine include a KRAS_Q61H mutation.

Exemplary epitope sequences that can be encoded by a cancer vaccine include an EGFR mutation, such as an EGFR_L858R mutation. Exemplary epitope sequences that can be encoded by a cancer vaccine include an an EGFR_L858R mutation.

Epitope sequences can include one or more subject-specific epitopes. Epitope sequences can include one or more subject-specific epitopes where the tumor of the subject has been sequenced to determine the subject-specific epitopes to be encoded by the cancer vaccine. Subject-specific epitopes can include at least 2 subject-specific epitopes, at least 10 subject-specific epitopes, at least 20 subject-specific epitopes, or between 2-20 subject-specific epitopes. Subject-specific epitopes can include at least 2 subject-specific epitopes. Subject-specific epitopes can include at least 10 subject-specific epitopes. Subject-specific epitopes can include at least 20 subject-specific epitopes. Subject-specific epitopes can include between 2-20 subject-specific epitopes.

Panels can further include additional tumor-informed polynucleotide probes that capture additional target sequences that are not encoded by a cancer vaccine, such as additional target sequences determined through cancer/tumor sequencing. For example, panels can include additional target sequences that have been predicted to be presented by a subject's HLA alleles but were not chosen for inclusion in a cancer vaccine. Panels can include additional target sequences additional target sequences determined through cancer/tumor sequencing that are known or considered to be associated with cancer. Additional target sequences can include at least 10 target sequences, at least 20 target sequences, at least 30 target sequences, at least 100 target sequences, between 10-500 target sequences, between 30-500 target sequences, between 100-500 target sequences, between 10-100 target sequences, between 30-100 target sequences, or between 100-100 target sequences. Additional target sequences can include at least 10 target sequences. Additional target sequences can include at least 20 target sequences. Additional target sequences can include at least 30 target sequences. Additional target sequences can include at least 100 target sequences. Additional target sequences can include between 10-500 target sequences. Additional target sequences can include between 30-500 target sequences. Additional target sequences can include between 100-500 target sequences. Additional target sequences can include between 10-100 target sequences. Additional target sequences can include between 30-100 target sequences. Additional target sequences can include between 100-100 target sequences.

Tumor-naïve polynucleotide probes can be configured to capture a target sequence that includes a sequence of interest including, but not limited to, a cancer-associated gene, an oncogene, a tumor-suppressor gene, an interferon-y signaling pathway gene, an antigen-processing pathway gene, and combinations thereof.

Oncogenes (also referred to as “driver” mutations) are genes generally considered or predicted to promote cancer, an typically are considered gain-of-function mutations (e.g., KRAS mutations). A panel can include probes specific for oncogenes, including “hotspots” within them, that include, but are not limited to, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20. A panel can include probes specific for oncogenes, including “hotspots” within them, that include each of ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20.

Tumor-suppressor genes are genes generally considered to monitor and/or control tumor-associated properties, such as cell division, where mutations can interfere with controlling such properties, and are typically considered loss-of-function mutations. A panel can include probes specific for tumor-suppressor genes, including “hotspots” within them, that include, but are not limited to, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3. A panel can include probes specific for tumor-suppressor genes, including “hotspots” within them, that include each of: TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3.

Interferon-y signaling pathway genes are genes involved in interferon-y signaling, such as JAK/STAT signaling pathway genes. A panel can include probes specific for interferon-y signaling pathway genes, including “hotspots” within them, that include, but are not limited to, IFNGR1, INFGR2, JAK1, JAK2, and STAT1. A panel can include probes specific for interferon-y signaling pathway genes, including “hotspots” within them, that include each of IFNGR1, INFGR2, JAK1, JAK2, and STAT1.

Antigen-processing pathway genes are genes involved in antigen processing and/or presentation (e.g., presentation by MHC), and can include monitoring for HLA loss of heterozygosity. A panel can include probes specific for antigen-processing pathway genes, including “hotspots” within them, that include, but are not limited to, B2M, HLA-A, HLA-B, HLA-C, HLA-E, TAP1, TAP2, NLRC5, CALR, CANX, PSMB2, and TAPBP. A panel can include probes specific for antigen-processing pathway genes, including “hotspots” within them, that include each of: B2M, HLA-A, HLA-B, HLA-C, HLA-E, TAP1, TAP2, NLRC5, CALR, CANX, PSMB2, and TAPBP.

Other mutations otherwise generally associated with cancer can also be monitored although not otherwise annotated, e.g., not yet annotated as an oncogene or tumor-suppressor. A panel can include probes specific for cancer-associated genes, including “hotspots” within them, that include, but are not limited to, ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2. A panel can include probes specific for cancer-associated genes, including “hotspots” within them, that include each of ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2.

A panel with tumor-naïve polynucleotide probes can be designed to capture each of a cancer-associated gene, an oncogene, a tumor-suppressor gene, an interferon-γ signaling pathway gene, and an antigen-processing pathway gene.

An exemplary, non-limiting, set of tumor-naïve polynucleotide probes include probes specific for genes, including “hotspots” within them, that include, but are not limited to, ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2, TAPBP, and combinations thereof.

An exemplary, non-limiting, set of tumor-naïve polynucleotide probes include probes specific for each of ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2.

An exemplary, non-limiting, set of tumor-naïve polynucleotide probes include probes specific for each of ABL1, AKT2, ALK, APC, AR, ATR, ATRX, BARD1, BCL6, BMPR1A, BRAF, BRCA1, BRCA2, BTK, CARD11, CCND1, CCND3, CDK12, CFH, CREBBP, CTNNB1, DDR2, DNMT3A, EGFR, EP300, ERBB2, ERBB3, ERCC2, ERCC5, EXT1, FANCA, FANCD2, FANCI, FANCM, FBXW7, FGF10, FGF6, FGFR1, FGFR3, FLI1, FLT1, FLT3, GNAS, HNF1A, HRAS, KDR, KIT, KRAS, MAGI1, MAP2K1, MAP2K2, MAX, MED12, MET, MLH1, MMAB, MSH3, MSH6, MTOR, NF1, NFE2L2, NOTCH1, NOTCH2, NOTCH3, NRAS, NRG1, NTRK1, NTRK3, PDGFRA, PDGFRB, PIK3CA, PIK3CG, PIK3R1, PMS2, PPARG, PROC, PTCH1, RAD54L, RAF1, RECQL4, RET, ROS1, SF3B1, SF3B2, SLX4, SMO, TERT promoter, TET2, TP53BP1, TSC1, TSC2, WRN, XPA, XPC, ZNF395, B2M, HLA-A, HLA-B, HLA-C, TAP1, TAP2, NLRC5, IFNGR1, INFGR2, JAK1, JAK2, TP53, PTEN, and ARID1A.

Probes in a panel can be designed to monitor the full coding-regions of select genes, e.g. through a series of overlapping probes across all exons of a specific gene. Probes in a panel can be designed to monitor particular regions or mutations (“hotspots”) within a gene. Probes in a panel can be designed to include two or more probes configured to capture a genomic region of interest (e.g., either a full coding region or a “hotspot”) associated-with cancer. Probes in a panel can be designed to include probes that include overlapping sequences of each other. As an illustrative, non-limiting examples overlapping probes can include a probe design of two probes each 90 nucleotides in length and shifted 20 bases from each other that can cover 110 bp for each target.

A probe panel can include at least 20 probes, at least 30 probes, at least 40 probes, at least 50 probes, at least 60 probes, at least 70 probes, at least 80 probes, at least 90 probes, at least 100 probes, at least 200 probes, at least 300 probes, at least 400 probes, or at least 500 probes. A probe panel can include at least 20 probes. A probe panel can include at least 30 probes. A probe panel can include at least 40 probes. A probe panel can include at least 50 probes. A probe panel can include at least 60 probes. A probe panel can include at least 70 probes. A probe panel can include at least 80 probes. A probe panel can include at least 90 probes. A probe panel can include at least 100 probes. A probe panel can include at least 200 probes. A probe panel can include at least 300 probes. A probe panel can include at least 400 probes. A probe panel can include at least 500 probes.

A probe panel can be configured to cover at least 100 kb, at least 300 kb, at least 300 kb, at least 400 kb, between 100-400 kb, between 200-400 kb, between 300-400 kb, between 100-500 kb, between 200-500 kb, between 300-500 kb, or between 340-400 kb of a subject's genome. A probe panel can be configured to cover at least 100 kb of a subject's genome. A probe panel can be configured to cover at least 300 kb of a subject's genome. A probe panel can be configured to cover at least 300 kb of a subject's genome. A probe panel can be configured to cover at least 400 kb of a subject's genome. A probe panel can be configured to cover between 100-400 kb of a subject's genome. A probe panel can be configured to cover between 200-400 kb of a subject's genome. A probe panel can be configured to cover between 300-400 kb of a subject's genome. A probe panel can be configured to cover between 100-500 kb of a subject's genome. A probe panel can be configured to cover between 200-500 kb of a subject's genome. A probe panel can be configured to cover between 300-500 kb of a subject's genome. A probe panel can be configured to cover between 340-400 kb of a subject's genome.

Tumor-naïve polynucleotide probes can include polynucleotide probes configured to capture sequences associated with a given cancer the subject is known to have or suspected of having, such as CRC or NSCLC. Tumor-naïve polynucleotide probes can include polynucleotide probes configured to capture sequences associated with CRC. Tumor-naïve polynucleotide probes can include polynucleotide probes configured to capture sequences associated with NSCLC. Tumor-naïve polynucleotide probes can include polynucleotide probes configured to capture sequences associated with GEA.

A panel can further include additional polynucleotide probes configured to capture sequences comprising polymorphisms (e.g., single-nucleotide polymorphisms “SNPs”) in the human population, where the sequences comprising polymorphisms are capable in combination of uniquely identifying (“fingerprinting”) a subject. Such sequences can be used, for example, if multiple subject samples are multiplexed for sequencing.

Hybridization typically refers to the process by which a strand of nucleic acid joins with a complementary strand through base pairing as known in the art. A nucleic acid is generally considered to selectively hybridize to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3^{rd} ed., Wiley & Sons 1995 and Sambrook et al., Molecular Cloning: A Laboratory Manual, Third Edition, 2001 Cold Spring Harbor, N.Y.). A hybridization protocol can occur at about 42° C. A hybridization buffer can include, but is not limited to, formamide, SSC, Denhardt's solution, SDS and/or denatured carrier DNA. A hybridization protocol can include washing steps in a buffer that can include SSC and SDS at 42° C. An illustrative, non-limiting hybridization protocol involves hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. Another illustrative, non-limiting example of high-stringency conditions includes hybridization overnight using custom-designed xGen Lockdown Probes and the xGen Hybridization and Wash kit (IDT), which involves hybridizing in xGen Hybridization Buffer, plus Hybridization Buffer Enhancer in a thermocycler at 95° C. for 30 seconds, followed by 65° C. for 4-16 hours; then washing once in xGen wash buffer once at room temperature; then washing twice in xGen Stringent Wash Buffer at 65° C.; and finally washing three times at room temperature in Wash Buffer 1, Wash Buffer 2, and Wash Buffer 3, respectively (per the manufacturer's instructions). One having ordinary skill in the art can recognize that the above non-limiting illustrative protocols can be optimized based on specific hybridization reactions.

Baits can be 80 to 150 base pairs (bp) in length, including 80 to 140, 80 to 130, 80 to 120, 80 to 110, 80 to 100, 80 to 90, 90 to 150, 90 to 140, 90 to 130, 90 to 120, 90 to 110, 90 to 100, 100 to 150, 100 to 140, 100 to 130,100 to 120, 100 to 110, 110 to 150,110 to 140, 110 to 130,110 to 120, 120 to 150, 120 to 140,120 to 130, 130 to 150, 130 to 140,140 to 150 bp in length. Baits can be 80 to 150 bp in length. Baits can be 80 to 140 bp in length. Baits can be 80 to 130 bp in length. Baits can be 80 to 120 bp in length. Baits can be 80 to 110 bp in length. Baits can be 80 to 100 bp in length. Baits can be 80 to 90 bp in length. Baits can be 90 to 150 bp in length. Baits can be 90 to 140 bp in length. Baits can be 90 to 130 bp in length. Baits can be 90 to 120 bp in length. Baits can be 90 to 110 bp in length. Baits can be 90 to 100 bp in length. Baits can be 100 to 150 bp in length. Baits can be 100 to 140 bp in length. Baits can be 100 to 130 bp in length. Baits can be 100 to 120 bp in length. Baits can be 100 to 110 bp in length. Baits can be 110 to 150 bp in length. Baits can be 110 to 140 bp in length. Baits can be 110 to 130 bp in length. Baits can be 110 to 120 bp in length. Baits can be 120 to 150 bp in length. Baits can be 120 to 140 bp in length. Baits can be 120 to 130 bp in length. Baits can be 130 to 150 bp in length. Baits can be 130 to 140 bp in length. Baits can be 140 to 150 bp in length.

Polynucleotide probes can include an affinity tag. Affinity tags are typically molecules that are capable of covalent linkage to a substrate molecule (e.g., a hybridization probe) and used for subsequent purification by binding of the tag to another surface or material with (e.g., a biotin tag binding to streptavidin resin). Enrichment of polynucleotides can occur by affinity purification or any other suitable method based on the affinity tag used. In some embodiments, an affinity tag is added to polynucleotide probes, enriching for the DNA molecules that hybridize with probes tagged with the affinity tag; and sequencing the enriched DNA molecules.

Polynucleotide probes (“baits”) can be biotinylated. Biotinylation refers to the covalent addition of a biotin moiety to the polynucleotide probes. A biotin moiety can include biotin or a biotin analogue, such as desthiobiotin, oxybiotin, 2-iminobiotin, diaminobiotin, biotin sulfoxide, biocytin, etc. Biotin moieties typically bind to streptavidin with an affinity of at least 10-8 M. Enrichment steps using biotinylated polynucleotide probes may be done using magnetic streptavidin beads, although other supports could be used including but not limited to microparticles, fibers, beads, and supports.

In an illustrative non-limiting example, enrichment can comprise steps of: (a) linking a biotin moiety to the oligonucleotide probes; (b) hybridizing biotinylated probes to cfDNA; (c) enriching for biotinylated DNA molecules by binding to a support that binds to biotin (e.g., streptavidin beads); (d) amplifying the enriched DNA using polymerase chain reaction; and (f) sequencing the amplified DNA to produce a plurality of sequence reads.

Multiple polynucleotide regions of interest can be selected for enrichment based on the specific disease or therapy being monitored. In cancer patients for example, sequence analysis of tumor genomic DNA can be used to identify tumor-specific mutations, which can be used to select regions of interest for disease monitoring.

Regions of interest can be enriched from cfDNA prior to sequencing. Regions of interest can also comprise polynucleotides encoding a coding region, which can include tumor exome polynucleotides.

Sequencing of cfDNA

Methods for sequencing of cfDNA are generally known to those skilled in the art. For example, general methods for sequencing cfDNA are described in US-2020/0277667-A1, which is herein incorporated by reference for all purposes. In general, any of the sequencing methods described herein can be used.

Sequencing of isolated cfDNA can comprise next-generation sequencing (NGS) or Sanger sequencing. The terms “next-generation sequencing” or “high-throughput sequencing”, as used herein, refer to the so-called parallelized sequencing-by-synthesis or sequencing-by-ligation platforms. NGS methods may also include nanopore sequencing methods or electronic-detection based methods NGS can comprise duplex sequencing, whole-exome sequencing, whole-genome sequencing, de novo sequencing, phased sequencing, targeted amplicon sequencing, or shotgun sequencing. NGS can be performed on platforms such as NovaSeq using 2×151 bp and 8 bp index reads. Other NGS platforms include but are not limited to Illumina HiSeq or MiSeq, Thermo PGM or Proton, the Pac Bio RS II or Sequel, Qiagen's Gene Reader, and the Oxford Nanopore MinION, or any other appropriate platform. Examples of such methods are described in Margulies et al. (Nature 2005 437:376-80); Ronaghi et al. (Analytical Biochemistry 1996 242:84-9); Shendure (Science 2005 309:1728); Imelfort et al. (Brief Bioinform. 2009 10:609-18); Fox et al. (Methods Mol Biol. 2009; 55379-108); Appleby et al. (Methods Mol Biol. 2009; 513:19-39) English (PloS One. 2012 7:e47768) and Morozova (Genomics. 2008 92:255-64), which are incorporated by reference for the general descriptions of the methods and the particular steps of the methods, including all starting products, reagents, and final products for each of the steps.

NGS can result in at least 10,000, at least 50,000, at least 100,000, at least 500,000, at least 1M, at least 10M, at least 100M, or at least 1B sequence reads. NGS can result in at least 10,000 sequence reads. NGS can result in at least 50,000 sequence reads. NGS can result in at least 100,000 sequence reads. NGS can result in at least 500,000 sequence reads. NGS can result in at least 1M sequence reads. NGS can result in at least 10M sequence reads. NGS can result in at least 100M sequence reads. NGS can result in at least 1B sequence reads. Sequence reads can be analyzed by a computer and, thus instructions for performing the steps can be set forth as programming that may be recorded in a suitable physical computer readable storage medium.

Whole library amplification can be performed on cfDNA, including enriched cfDNA, using kits such as KAPA HiFi HotStart ReadyMix and NEBNext Multiple Oligos for Illumina.

As an illustrative non-limiting example of the process described herein, whole blood can be collected for a given subject or collected from a subject with cancer undergoing therapy and cfDNA can be isolated from the whole blood. Sequencing of DNA from a diseased tissue (e.g., a cancer-disease tissue, such as from a tumor biopsy) can be used to identify subject-specific and/or tumor-specific mutations. Subject-specific and/or tumor-specific mutations can be used to design a library of biotinylated polynucleotide probes and/or guide selection of biotinylated polynucleotide probes to enrich polynucleotide regions of interest from subject cfDNA specific to a subject's cancer/tumor. Duplex sequencing adaptors can be ligated to the cfDNA, which can then be analyzed by duplex sequencing to measure the frequency of all variant alleles probed.

### Sequencing Adaptors and Duplex Sequencing

In general, for methods involving next-generation sequencing, adaptors are ligated to the cfDNA to facilitate sequencing. The terms “sequencing adaptor” or “adaptor” refer to oligonucleotides that are ligated onto the ends of polynucleotides from prepared libraries prior to sequencing (e.g., a fragmented cfDNA library of polynucleotide regions of interest). Adaptor ligation can be performed on fragmented, end-repaired DNA using 5-mer non-random unique molecular identifiers (IDT, Coralville, Iowa).

Sequencing adaptors can be configured for duplex sequencing. In general, duplex sequencing allows for independent tracking during sequencing of both strands of individual DNA molecules. The paired sequences can be compared to reduce sequencing errors by excluding variations that do not occur on both DNA strands. Adaptors configured for duplex sequencing can include xGen UMI adaptors (IDT). General descriptions of sequencing adaptors for duplex sequencing and uses thereof are described in US 2017/0211140 A1, which is hereby incorporated by reference for all purposes.

### Read Depth

Sequencing read depth (presented as X-fold, e.g., 1000×, read depth and referred to in some instances as sequencing read coverage) as used herein refers to the level of coverage of reads (e.g., number of unique reads), after detection and removal of duplicate reads (e.g., PCR duplicate reads). In general, greater sequencing read depth correlates with greater variant detection reliability. For example, reliable detection of a variant, e.g., a point mutation, that appears at a frequency of greater than 5% and up to 10, 15 or 20% can typically need >200× sequencing depth to ensure high detection reliability.

Sequencing read depth can be the read depth for an individual mutation. Sequencing read depth for an individual mutation can be at least 1000×. Sequencing read depth for an individual mutation can be at least 1500×, at least 2000×, at least 2500×, at least 3000×, at least 3500×, at least 4000×, at least 4500×, or at least 5000×. Sequencing read depth for an individual mutation can be an at least 1500×. Sequencing read depth for an individual mutation can be at least 2000×. Sequencing read depth for an individual mutation can be at least 2500×. Sequencing read depth for an individual mutation can be at least 3000×. Sequencing read depth for an individual mutation can be at least 3500×. Sequencing read depth for an individual mutation can be at least 4000×. Sequencing read depth for an individual mutation can be at least 4500×. Sequencing read depth for an individual mutation can be at least 5000×. Sequencing read depth for an individual mutation can range from 1000× to 5000×, including 1000× to 4000×, 1000× to 3000×, 1000× to 2000×, 2000× to 5000×, 2000× to 4000×, 2000× to 3000×, 3000× to 5000×, 3000× to 4000×, and 4000× to 5000×. Sequencing read depth for an individual mutation can range from 1000× to 5000×. Sequencing read depth for an individual mutation can range from 1000× to 4000×. Sequencing read depth for an individual mutation can range from 1000× to 3000×. Sequencing read depth for an individual mutation can range from 1000× to 2000×. Sequencing read depth for an individual mutation can range from 2000× to 5000×. Sequencing read depth for an individual mutation can range from 2000× to 4000×. Sequencing read depth for an individual mutation can range from 2000× to 3000×. Sequencing read depth for an individual mutation can range from 3000× to 5000×. Sequencing read depth for an individual mutation can range from 3000× to 4000×. Sequencing read depth for an individual mutation can range from 4000× to 5000×. Sequencing read depth for an individual mutation can range from at least 100× to 1000×.

Sequencing read depth can be duplex read depth. Sequencing read depth can be duplex read depth for an individual mutation. Duplex read depth for an individual mutation can be at least 1000×. Duplex read depth for an individual mutation can be at least 1500×, at least 2000×, at least 2500×, at least 3000×, at least 3500×, at least 4000×, at least 4500×, or at least 5000×. Duplex read depth for an individual mutation can be at least 1500×. Duplex read depth for an individual mutation can be at least 2000×. Duplex read depth for an individual mutation can be at least 2500×. Duplex read depth for an individual mutation can be at least 3000×. Duplex read depth for an individual mutation can be at least 3500×. Duplex read depth for an individual mutation can be at least 4000×. Duplex read depth for an individual mutation can be at least 4500×. Duplex read depth for an individual mutation can be at least 5000×. Duplex read depth for an individual mutation can range from 1000× to 5000×, including 1000× to 4000×, 1000× to 3000×, 1000× to 2000×, 2000× to 5000×, 2000× to 4000×, 2000× to 3000×, 3000× to 5000×, 3000× to 4000×, and 4000× to 5000×. Duplex read depth for an individual mutation can range from 1000× to 5000×. Duplex read depth for an individual mutation can range from 1000× to 4000×. Duplex read depth for an individual mutation can range from 1000× to 3000×. Duplex read depth for an individual mutation can range from 1000× to 2000×. Duplex read depth for an individual mutation can range from 2000× to 5000×. Duplex read depth for an individual mutation can range from 2000× to 4000×. Duplex read depth for an individual mutation can range from 2000× to 3000×. Duplex read depth for an individual mutation can range from 3000× to 5000×. Duplex read depth for an individual mutation can range from 3000× to 4000×. Duplex read depth for an individual mutation can range from 4000× to 5000×. Duplex read depth for an individual mutation can range from at least 100× to 1000×.

Sequencing read depth can be the mean read depth. Mean read depth refers to the mean sequencing depth of a plurality of polynucleotide regions of interest (e.g., a cancer exome and/or regions of interest targeted for enrichment, such as by any of the tumor-informed/tumor-naïve combination panels described herein, and/or by baits for regions having subject-specific and tumor-specific variants). Mean read depth can be the mean read depth of a cancer exome. Mean read depth can be the mean read depth of regions of interest targeted for enrichment by any of the tumor-informed/tumor-naïve combination panels described herein. Mean read depth can be the mean read depth of previously identified regions of interest having subject-specific and/or tumor-specific mutations. Mean read depth can be the mean read depth of enriched cfDNA. Mean read depth can be the mean read depth of cfDNA enriched by baits for regions having subject-specific and tumor-specific variants.

Mean read depth can be at least 1000×. Mean read depth can be at least 1500×, at least 2000×, at least 2500×, at least 3000×, at least 3500×, at least 4000×, at least 4500×, or at least 5000×. Mean read depth can be at least 1500×. Mean read depth can be at least 2000×. Mean read depth can be at least 2500×. Mean read depth can be at least 3000×. Mean read depth can be at least 3500×. Mean read depth can be at least 4000×. Mean read depth can be at least 4500×. Mean read depth can be at least 5000×. Mean read depth can range from 1000× to 5000×, including 1000× to 4000×, 1000× to 3000×, 1000× to 2000×, 2000× to 5000×, 2000× to 4000×, 2000× to 3000×, 3000× to 5000×, 3000× to 4000×, and 4000× to 5000×. Mean read depth can range from 1000× to 5000×. Mean read depth can range from 1000× to 4000×. Mean read depth can range from 1000× to 3000×. Mean read depth can range from 1000× to 2000×. Mean read depth can range from 2000× to 5000×. Mean read depth can range from 2000× to 4000×. Mean read depth can range from 2000× to 3000×. Mean read depth can range from 3000× to 5000×. Mean read depth can range from 3000× to 4000×. Mean read depth can range from 4000× to 5000×. Mean read depth can range from at least 100× to 1000×.

Mean read depth can be mean duplex read depth. Mean duplex read depth can be at least 1000×. Mean duplex read depth can be at least 1500×, at least 2000×, at least 2500×, at least 3000×, at least 3500×, at least 4000×, at least 4500×, or at least 5000×. Mean duplex read depth can be at least 1500×. Mean duplex read depth can be at least 2000×. Mean duplex read depth can be at least 2500×. Mean duplex read depth can be at least 3000×. Mean duplex read depth can be at least 3500×. Mean duplex read depth can be at least 4000×. Mean duplex read depth can be at least 4500×. Mean duplex read depth can be at least 5000×. Mean duplex read depth can range from 1000× to 5000×, including 1000× to 4000×, 1000× to 3000×, 1000× to 2000×, 2000× to 5000×, 2000× to 4000×, 2000× to 3000×, 3000× to 5000×, 3000× to 4000×, and 4000× to 5000×. Mean duplex read depth can range from 1000× to 5000×. Mean duplex read depth can range from 1000× to 4000×. Mean duplex read depth can range from 1000× to 3000×. Mean duplex read depth can range from 1000× to 2000×. Mean duplex read depth can range from 2000× to 5000×. Mean duplex read depth can range from 2000× to 4000×. Mean duplex read depth can range from 2000× to 3000×. Mean duplex read depth can range from 3000× to 5000×. Mean duplex read depth can range from 3000× to 4000×. Mean duplex read depth can range from 4000× to 5000×. Mean duplex read depth can range from at least 100× to 1000×.

### Multiplexed Analysis

Methods described herein include multiplex arrays that can sequence (“detect”) multiple polynucleotide regions of interest from a cfDNA sample. A cfDNA sample can comprise ctDNA containing one or more mutant alleles encoding genes in the tumor exome. One or more polynucleotide regions of interest can be selectively enriched through designing baits to target the one or more polynucleotide regions of interest. One or more polynucleotide regions of interest can be selectively enriched through designing baits to target the one or more polynucleotide regions of interest from a tumor exome. One or more polynucleotide regions of interest can be selectively enriched through designing baits to target the one or more polynucleotide regions of interest from a tumor exome known or suspected of having subject and tumor-specific mutations.

One or more polynucleotide regions or interest can comprise 10 or more polynucleotide regions of interest. One or more polynucleotide regions or interest can comprise 20 polynucleotide regions of interest. One or more polynucleotide regions or interest can comprise 30 or more polynucleotide regions of interest. One or more polynucleotide regions or interest can comprise 40 or more polynucleotide regions of interest. One or more polynucleotide regions or interest can comprise 50 or more polynucleotide regions of interest. One or more polynucleotide regions or interest can comprise 60 or more polynucleotide regions of interest. One or more polynucleotide regions or interest can comprise 70 or more polynucleotide regions of interest. One or more polynucleotide regions or interest can comprise 80 or more polynucleotide regions of interest. One or more polynucleotide regions or interest can comprise 90 or more polynucleotide regions of interest. One or more polynucleotide regions of interest can comprise 100 or more polynucleotide regions of interest. One or more polynucleotide regions of interest can comprise 150 or more polynucleotide regions of interest. One or more polynucleotide regions of interest can comprise 200 or more polynucleotide regions of interest. One or more polynucleotide regions of interest can comprise 250 or more polynucleotide regions of interest. One or more polynucleotide regions of interest can comprise 300 or more polynucleotide regions of interest. One or more polynucleotide regions of interest can comprise 400 or more polynucleotide regions of interest. One or more polynucleotide regions of interest can comprise 500 or more polynucleotide regions of interest. One or more polynucleotide regions of interest can comprise 600 or more polynucleotide regions of interest. One or more polynucleotide regions of interest can comprise 700 or more polynucleotide regions of interest. One or more polynucleotide regions of interest can comprise 800 or more polynucleotide regions of interest. Or One or more polynucleotide regions of interest can comprise 900 or more polynucleotide regions of interest.

One or more polynucleotide regions of interest can comprise at least 10% of polynucleotide regions of interest corresponding to mutations present in a tumor exome of the subject (in other words, at least 10% of all subject and tumor-specific mutations associated with a tumor exome). One or more polynucleotide regions of interest can comprise at least 20% of polynucleotide regions of interest corresponding to mutations present in a tumor exome of the subject. One or more polynucleotide regions of interest can comprise at least 30% of polynucleotide regions of interest corresponding to mutations present in a tumor exome of the subject. One or more polynucleotide regions of interest can comprise at least 40% of polynucleotide regions of interest corresponding to mutations present in a tumor exome of the subject. One or more polynucleotide regions of interest can comprise at least 50% of polynucleotide regions of interest corresponding to mutations present in a tumor exome of the subject. One or more polynucleotide regions of interest can comprise at least 60% of polynucleotide regions of interest corresponding to mutations present in a tumor exome of the subject. One or more polynucleotide regions of interest can comprise at least 70% of polynucleotide regions of interest corresponding to mutations present in a tumor exome of the subject. One or more polynucleotide regions of interest can comprise at least 80% of polynucleotide regions of interest corresponding to mutations present in a tumor exome of the subject. One or more polynucleotide regions of interest can comprise at least 90% of polynucleotide regions of interest corresponding to mutations present in a tumor exome of the subject. The one or more polynucleotide regions of interest can comprise at least 95% of polynucleotide regions of interest corresponding to mutations present in a tumor exome of the subject. The one or more polynucleotide regions of interest can comprise at least 96% of polynucleotide regions of interest corresponding to mutations present in a tumor exome of the subject. The one or more polynucleotide regions of interest can comprise at least 97% of polynucleotide regions of interest corresponding to mutations present in a tumor exome of the subject. The one or more polynucleotide regions of interest can comprise at least 98% of polynucleotide regions of interest corresponding to mutations present in a tumor exome of the subject. The one or more polynucleotide regions of interest can comprise at least 99% of polynucleotide regions of interest corresponding to mutations present in a tumor exome of the subject. The one or more polynucleotide regions of interest can comprise at least 99.5% of polynucleotide regions of interest corresponding to mutations present in a tumor exome of the subject. The one or more polynucleotide regions of interest can comprise at least 99.9% of polynucleotide regions of interest corresponding to mutations present in a tumor exome of the subject. The one or more polynucleotide regions of interest can comprise 100% of polynucleotide regions of interest corresponding to mutations present in a tumor exome of the subject.

Mutations can comprise but are not limited to a point mutation, a frameshift mutation, a non-frameshift mutation, a deletion mutation, an insertion mutation, a splice variant, a genomic rearrangement, a proteasome-generated spliced antigen, or combinations thereof. Mutations can comprise at least one alteration that makes a peptide sequence encoded by the cfDNA distinct from the corresponding peptide sequence encoded by the wild-type germline nucleic acid sequence of the subject. Mutations can consist of coding mutations comprising at least one alteration that makes a peptide sequence encoded by the cfDNA distinct from the corresponding peptide sequence encoded by the wild-type germline nucleic acid sequence of the subject. One or more mutations can include 10 or more, 20 or more, 30 or more, 40 or more, 50 or more, 60 or more, 70 or more, 80 or more, or 90 or more mutations. One or more mutations can include 100 or more, 150 or more, 200 or more, 250 or more, 300 or more, 400 or more, 500 or more, 600 or more, 700 or more, 800 or more, or 900 or more mutations. Mutations can be associated with a tumor exome. One or more mutations can include at least 10%, at least 20%, at least 30%, at least 40%, at least 50%, at least 60%, at least 70%, at least 80%, or at least 90% of mutations present in a tumor exome of the subject. One or more mutations can include at least 95%, at least 96%, at least 97%, at least 98%, at least 99%, at least 99.5%, at least 99.9%, 100% of mutations present in a tumor exome of the subject.

### Target Coverage

Target coverage (typically presented as a percentage) as used herein refers to the proportion of a polynucleotide region or plurality of regions that is sequenced (e.g., regions represented in a sequencing data set to at least some read depth). In general, target coverage is described as a proportion of a desired region or plurality of regions to be covered (e.g., a plurality of polynucleotide regions of interest). For example, target coverage can be the proportion of a whole genome, an exome, a cancer genome, a cancer exome, and/or an enriched region (e.g., a cancer exome and/or regions of interest targeted for enrichment, such as by any of the tumor-informed/tumor-naïve combination panels described herein, and/or by baits for regions having subject-specific and tumor-specific variants).

Target coverage can be the proportion of a tumor and/or cancer exome of a subject that is sequenced. Target coverage can be at least 10% of a tumor and/or cancer exome. Target coverage can be at least 20% of a tumor and/or cancer exome. Target coverage can be at least 30% of a tumor and/or cancer exome. Target coverage can be at least 40% of a tumor and/or cancer exome. Target coverage can be at least 50% of a tumor and/or cancer exome. Target coverage can be at least 60% of a tumor and/or cancer exome. Target coverage can be at least 70% of a tumor and/or cancer exome. Target coverage can be at least 80% of a tumor and/or cancer exome. Target coverage can be at least 90% of a tumor and/or cancer exome. Target coverage can be at least 95% of a tumor and/or cancer exome. Target coverage can be at least 96% of a tumor and/or cancer exome. Target coverage can be at least 97% of a tumor and/or cancer exome. Target coverage can be at least 98% of a tumor and/or cancer exome. Target coverage can be at least 99% of a tumor and/or cancer exome. Target coverage can be at least 99.5% of a tumor and/or cancer exome. Target coverage can be at least 99.9% of a tumor and/or cancer exome. Target coverage can be 100% of a tumor and/or cancer exome.

Target coverage can be the proportion of polynucleotide regions of interest that is sequenced. Target coverage can be at least 10% of polynucleotide regions of interest. Target coverage can be at least 20% of polynucleotide regions of interest. Target coverage can be at least 30% of polynucleotide regions of interest. Target coverage can be at least 40% of polynucleotide regions of interest. Target coverage can be at least 50% of polynucleotide regions of interest. Target coverage can be at least 60% of polynucleotide regions of interest. Target coverage can be at least 70% of polynucleotide regions of interest. Target coverage can be at least 80% of polynucleotide regions of interest. Target coverage can be at least 90% of polynucleotide regions of interest. Target coverage can be at least 95% of polynucleotide regions of interest. Target coverage can be at least 96% of polynucleotide regions of interest. Target coverage can be at least 97% of polynucleotide regions of interest. Target coverage can be at least 98% of polynucleotide regions of interest. Target coverage can be at least 99% of polynucleotide regions of interest. Target coverage can be at least 99.5% of polynucleotide regions of interest. Target coverage can be at least 99.9% of polynucleotide regions of interest. Target coverage can be 100% of polynucleotide regions of interest.

Target coverage can be the proportion of polynucleotide regions of interest targeted for enrichment that is sequenced (e.g., a cancer exome and/or regions of interest targeted for enrichment, such as by any of the tumor-informed/tumor-naïve combination panels described herein, and/or by baits for regions having subject-specific and tumor-specific variants). Target coverage can be at least 10% of polynucleotide regions of interest targeted for enrichment. Target coverage can be at least 20% of polynucleotide regions of interest targeted for enrichment. Target coverage can be at least 30% of polynucleotide regions of interest targeted for enrichment. Target coverage can be at least 40% of polynucleotide regions of interest targeted for enrichment. Target coverage can be at least 50% of polynucleotide regions of interest targeted for enrichment. Target coverage can be at least 60% of polynucleotide regions of interest targeted for enrichment. Target coverage can be at least 70% of polynucleotide regions of interest targeted for enrichment. Target coverage can be at least 80% of polynucleotide regions of interest targeted for enrichment. Target coverage can be at least 90% of polynucleotide regions of interest targeted for enrichment. Target coverage can be at least 95% of polynucleotide regions of interest targeted for enrichment. Target coverage can be at least 96% of polynucleotide regions of interest targeted for enrichment. Target coverage can be at least 97% of polynucleotide regions of interest targeted for enrichment. Target coverage can be at least 98% of polynucleotide regions of interest targeted for enrichment. Target coverage can be at least 99% of polynucleotide regions of interest targeted for enrichment. Target coverage can be at least 99.5% of polynucleotide regions of interest targeted for enrichment. Target coverage can be at least 99.9% of polynucleotide regions of interest targeted for enrichment. Target coverage can be 100% of polynucleotide regions of interest targeted for enrichment.

Target coverage can be the proportion of polynucleotide regions targeted for enrichment by any of the tumor-informed/tumor-naïve combination panels described herein.

Target coverage can be the proportion of polynucleotide regions of interest that is sequenced that corresponds to mutations present in a tumor and/or cancer exome of a subject. Target coverage can be at least 10% of all polynucleotide regions of interest corresponding to mutations present in a tumor and/or cancer exome of a subject (e.g., coverage is at least 10% of all subject-specific and tumor-specific mutations associated with a tumor and/or cancer exome). Target coverage can be at least 20% of all polynucleotide regions of interest corresponding to mutations present in a tumor and/or cancer exome of a subject. Target coverage can be at least 30% of all polynucleotide regions of interest corresponding to mutations present in a tumor and/or cancer exome of a subject. Target coverage can be at least 40% of all polynucleotide regions of interest corresponding to mutations present in a tumor and/or cancer exome of a subject. Target coverage can be at least 50% of all polynucleotide regions of interest corresponding to mutations present in a tumor and/or cancer exome of a subject. Target coverage can be at least 60% of all polynucleotide regions of interest corresponding to mutations present in a tumor and/or cancer exome of a subject. Target coverage can be at least 70% of all polynucleotide regions of interest corresponding to mutations present in a tumor and/or cancer exome of a subject. Target coverage can be at least 80% of all polynucleotide regions of interest corresponding to mutations present in a tumor and/or cancer exome of a subject. Target coverage can be at least 90% of all polynucleotide regions of interest corresponding to mutations present in a tumor and/or cancer exome of a subject. Target coverage can be at least 95% of all polynucleotide regions of interest corresponding to mutations present in a tumor and/or cancer exome of a subject. Target coverage can be at least 96% of all polynucleotide regions of interest corresponding to mutations present in a tumor and/or cancer exome of a subject. Target coverage can be at least 97% of all polynucleotide regions of interest corresponding to mutations present in a tumor and/or cancer exome of a subject. Target coverage can be at least 98% of all polynucleotide regions of interest corresponding to mutations present in a tumor and/or cancer exome of a subject. Target coverage can be at least 99% of all polynucleotide regions of interest corresponding to mutations present in a tumor and/or cancer exome of a subject. Target coverage can be at least 99.5% of all polynucleotide regions of interest corresponding to mutations present in a tumor and/or cancer exome of a subject. Target coverage can be at least 99.9% of all polynucleotide regions of interest corresponding to mutations present in a tumor and/or cancer exome of a subject. Target coverage can be 100% of all polynucleotide regions of interest corresponding to mutations present in a tumor and/or cancer exome of a subject.

Target coverage can be the proportion of a tumor and/or cancer genome of a subject that is sequenced. Target coverage can be at least 10% of a tumor and/or cancer genome. Target coverage can be at least 20% of a tumor and/or cancer genome. Target coverage can be at least 30% of a tumor and/or cancer genome. Target coverage can be at least 40% of a tumor and/or cancer genome. Target coverage can be at least 50% of a tumor and/or cancer genome. Target coverage can be at least 60% of a tumor and/or cancer genome. Target coverage can be at least 70% of a tumor and/or cancer genome. Target coverage can be at least 80% of a tumor and/or cancer genome. Target coverage can be at least 90% of a tumor and/or cancer genome. Target coverage can be at least 95% of a tumor and/or cancer genome. Target coverage can be at least 96% of a tumor and/or cancer genome. Target coverage can be at least 97% of a tumor and/or cancer genome. Target coverage can be at least 98% of a tumor and/or cancer genome. Target coverage can be at least 99% of a tumor and/or cancer genome. Target coverage can be at least 99.5% of a tumor and/or cancer genome. Target coverage can be at least 99.9% of a tumor and/or cancer genome. Target coverage can be 100% of a tumor and/or cancer genome.

Target coverage can be the percentage of regions of interest that are sequenced and where the sequenced regions of interest exceed a particular read depth. Target coverage can be the percentage of regions of interest that are sequenced and where the sequenced regions of interest have a read depth of at least 1000×. Target coverage can be the percentage of regions of interest that are sequenced and where the sequenced regions of interest have a read depth of at least 1500×, at least 2000×, at least 2500×, at least 3000×, at least 3500×, at least 4000×, at least 4500×, or at least 5000×. Target coverage can be the percentage of regions of interest that are sequenced and where the sequenced regions of interest have a read depth of at least 1500×. Target coverage can be the percentage of regions of interest that are sequenced and where the sequenced regions of interest have a read depth of at least 2000×. Target coverage can be the percentage of regions of interest that are sequenced and where the sequenced regions of interest have a read depth of at least 2500×. Target coverage can be the percentage of regions of interest that are sequenced and where the sequenced regions of interest have a read depth of at least 3000×. Target coverage can be the percentage of regions of interest that are sequenced and where the sequenced regions of interest have a read depth of at least 3500×. Target coverage can be the percentage of regions of interest that are sequenced and where the sequenced regions of interest have a read depth of at least 4000×. Target coverage can be the percentage of regions of interest that are sequenced and where the sequenced regions of interest have a read depth of at least 4500×. Target coverage can be the percentage of regions of interest that are sequenced and where the sequenced regions of interest have a read depth of at least 5000×.

Target coverage can be the percentage of regions of interest that are sequenced and where the sequenced regions of interest exceed a particular mean read depth. Target coverage can be the percentage of regions of interest that are sequenced and where the sequenced regions of interest have a mean read depth of at least 1000×. Target coverage can be the percentage of regions of interest that are sequenced and where the sequenced regions of interest have a mean read depth of at least 1500×, at least 2000×, at least 2500×, at least 3000×, at least 3500×, at least 4000×, at least 4500×, or at least 5000×. Target coverage can be the percentage of regions of interest that are sequenced and where the sequenced regions of interest have a mean read depth of at least 1500×. Target coverage can be the percentage of regions of interest that are sequenced and where the sequenced regions of interest have a mean read depth of at least 2000×. Target coverage can be the percentage of regions of interest that are sequenced and where the sequenced regions of interest have a mean read depth of at least 2500×. Target coverage can be the percentage of regions of interest that are sequenced and where the sequenced regions of interest have a mean read depth of at least 3000×. Target coverage can be the percentage of regions of interest that are sequenced and where the sequenced regions of interest have a mean read depth of at least 3500×. Target coverage can be the percentage of regions of interest that are sequenced and where the sequenced regions of interest have a mean read depth of at least 4000×. Target coverage can be the percentage of regions of interest that are sequenced and where the sequenced regions of interest have a mean read depth of at least 4500×. Target coverage can be the percentage of regions of interest that are sequenced and where the sequenced regions of interest have a mean read depth of at least 5000×.

Target coverage can be the percentage of regions of interest that are sequenced and where the sequenced regions of interest exceed a particular duplex read depth. Target coverage can be the percentage of regions of interest that are sequenced and where the sequenced regions of interest have a duplex read depth of at least 1000×. Target coverage can be the percentage of regions of interest that are sequenced and where the sequenced regions of interest have a duplex read depth of at least 1500×, at least 2000×, at least 2500×, at least 3000×, at least 3500×, at least 4000×, at least 4500×, or at least 5000×. Target coverage can be the percentage of regions of interest that are sequenced and where the sequenced regions of interest have a duplex read depth of at least 1500×. Target coverage can be the percentage of regions of interest that are sequenced and where the sequenced regions of interest have a duplex read depth of at least 2000×. Target coverage can be the percentage of regions of interest that are sequenced and where the sequenced regions of interest have a duplex read depth of at least 2500×. Target coverage can be the percentage of regions of interest that are sequenced and where the sequenced regions of interest have a duplex read depth of at least 3000×. Target coverage can be the percentage of regions of interest that are sequenced and where the sequenced regions of interest have a duplex read depth of at least 3500×. Target coverage can be the percentage of regions of interest that are sequenced and where the sequenced regions of interest have a duplex read depth of at least 4000×. Target coverage can be the percentage of regions of interest that are sequenced and where the sequenced regions of interest have a duplex read depth of at least 4500×. Target coverage can be the percentage of regions of interest that are sequenced and where the sequenced regions of interest have a duplex read depth of at least 5000×.

Target coverage can be the percentage of regions of interest that are sequenced and where the sequenced regions of interest exceed a particular mean duplex read depth. Target coverage can be the percentage of regions of interest that are sequenced and where the sequenced regions of interest have a mean duplex read depth of at least 1000×. Target coverage can be the percentage of regions of interest that are sequenced and where the sequenced regions of interest have a mean duplex read depth of at least 1500×, at least 2000×, at least 2500×, at least 3000×, at least 3500×, at least 4000×, at least 4500×, or at least 5000×.

Target coverage can be at least 10% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 20% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 30% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 40% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 50% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 60% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 70% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 80% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 90% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 95% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 96% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 97% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 98% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 99% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 99.5% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be at least 99.9% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×. Target coverage can be 100% of regions of interest and where the sequenced regions of interest have a read depth or mean read depth of at least 1000×, at least 1500×, at least 2000×, at least 2500×, at least 3000×, at least 3500×, at least 4000×, at least 4500×, or at least 5000×.

Target coverage can be at least 10% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 20% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 30% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 40% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 50% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 60% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 70% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 80% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 90% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 95% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 96% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 97% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 98% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 99% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 99.5% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be at least 99.9% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×. Target coverage can be 100% of regions of interest and where the sequenced regions of interest have a duplex read depth or mean duplex read depth of at least 1000×, at least 1500×, at least 2000×, at least 2500×, at least 3000×, at least 3500×, at least 4000×, at least 4500×, or at least 5000×.

Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 1000×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 1500×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 2000×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 2500×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 3000×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 3500×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 4000×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 4500×. Target coverage can be at least 50%, 55%, 60%, 65%, 70%, 75%, 80%, 85%, 90%, 95%, 96%, 97%, 98%, or 99% of regions of interest and where the sequenced regions of interest have a read depth or mean read depth of at least 5000×.

Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 1000×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 1500×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 2000×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 2500×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 3000×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 3500×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 4000×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 4500×. Target coverage can be at least 50%, 55%, 60%, 65%, 70%, 75%, 80%, 85%, 90%, 95%, 96%, 97%, 98%, or 99% of regions of interest and where the sequenced regions of interest have a duplex read depth or mean duplex read depth of at least 5000×.

### Assessment

Following sequencing, sequence reads can be analyzed to provide a quantitative determination of the frequency of variant alleles (also referred to as mutant allele frequency) within the cfDNA of a subject. Methods for quantifying sequencing reads and variant allele frequencies (VAF) are known to those skilled in the art. Computational programs for sequencing analysis and VAF, include, but are not limited to, BWA-MEM (Durbin et al, Bioinformatics, 2010), fgbio toolkit (Fulcrum Genomics), and freebayes (Marth et al, arXiv 2012), each of which is herein incorporated by reference for all purposes. In general, frequency of one or more mutations in a subject's cfDNA (e.g., VAF) is presented as the percentage of mutation specific sequencing reads relative to reads of wild-type germline nucleic acid sequences of the subject. For example, mutational frequency can be determined by counting the reads of a specific variant allele in comparison to total cfDNA counts for samples taken from a subject. Additionally, VAF assessments can be combined with cfDNA concentration in plasma (e.g., ng/ml) to estimate tumor genome concentrations in plasma (see Bos, et al Molecular Oncology (2020) doi: 10.1002/1878-0261.12827 and Reinert et al, JAMA Oncol. 2019; 5(8):1124-1131. Doi:10.1001/jamaoncol.2019.0528, each herein incorporated by reference for all purposes).

Following determination of the frequency of one or more mutations (or alternatively estimated tumor genomes per ml of plasma) in a subject's cfDNA (e.g., VAF), mutational frequency or estimated tumor genome content can then be assessed to characterize various disease or subject attributes, such as a status of a disease of a subject, efficacy of a therapy, or combinations thereof. Mutational frequency

Assessment can be done, for example, to assess disease status of a subject, such as assessing tumor burden of a subject. Assessment of tumor burden can be used in various applications, such as part of disease diagnosis, disease prognosis, disease prediction, and/or monitoring of disease progression. Assessment of disease progression can be done by comparing mutational frequency in samples taken from a subject at various timepoints. Changes in mutational frequency can be relative to a fixed timepoint, e.g., a baseline mutational frequency such as the mutational frequency determined on the first day of a therapy regimen.

An increase in mutational frequency from cfDNA mutation analysis of a first sample collected (e.g., an earlier longitudinal sample) relative to mutational frequency from cfDNA mutation analysis of a second sample (e.g., an later longitudinal sample) can be assessed as disease progression, unresponsiveness to therapy, and/or disease recurrence. A decrease in mutational frequency from cfDNA mutation analysis of a first sample collected (e.g., an earlier longitudinal sample) relative to mutational frequency from cfDNA mutation analysis of a second sample (e.g., an later longitudinal sample) can be assessed as a response. A response can be either a complete response (CR) or a partial response (PR). An increase in frequency of mutations in post-therapy cfDNA relative to pre-therapy cfDNA can indicate an increased likelihood that tumor burden of the subject is increasing. A decrease or maintenance of frequency of mutations in post-therapy cfDNA relative to pre-therapy cfDNA can indicate an increased likelihood that tumor burden of the subject is decreasing or stable.

An increase in mutational frequency (or alternatively estimated tumor genomes per ml of plasma) over time can be assessed as disease progression and/or recurrence. An increase in mutational frequency can be an at least 10%, at least 20%, at least 30%, at least 40%, at least 50%, at least 60%, at least 70%, at least 80%, at least 90%, or at least 100% relative increase in mutational frequency between timepoints to be assessed as progression and/or recurrence. An increase in mutational frequency can be an at least 2-fold, at least 3-fold, at least 4-fold, at least 5-fold, at least 6-fold, at least 7-fold, at least 8-fold, at least 9-fold, or at least 10-fold relative increase in mutational frequency between timepoints to be assessed as progression and/or recurrence. An increase in mutational frequency can be an at least 20-fold, at least 30-fold, at least 40-fold, at least 50-fold, at least 60-fold, at least 70-fold, at least 80-fold, at least 90-fold, or at least 100-fold relative increase in mutational frequency between timepoints to be assessed as progression and/or recurrence.

A decrease in mutational frequency (or alternatively estimated tumor genomes per ml of plasma) over time can be assessed as disease remission. A decrease in mutational frequency can be an at least 10%, at least 20%, at least 30%, at least 40%, at least 50%, at least 60%, at least 70%, at least 80%, at least 90%, or at least 100% relative increase in mutational frequency between timepoints to be assessed as remission. A decrease in mutational frequency can be an at least 2-fold, at least 3-fold, at least 4-fold, at least 5-fold, at least 6-fold, at least 7-fold, at least 8-fold, at least 9-fold, or at least 10-fold relative increase in mutational frequency between timepoints to be assessed as remission. A decrease in mutational frequency can be an at least 20-fold, at least 30-fold, at least 40-fold, at least 50-fold, at least 60-fold, at least 70-fold, at least 80-fold, at least 90-fold, or at least 100-fold relative increase in mutational frequency between timepoints to be assessed as remission. A decrease in mutational frequency can be to an undetectable level of mutations in the cfDNA to be assessed as remission, e.g., assessed as a complete remission.

Assessment can be done to assess de novo mutation status of a subject, such as assessing whether a subject's cancer or tumor develops an tumor evasion mutant (e.g., see “Cancer Monitoring”). An increase in mutational frequency from cfDNA mutation analysis of a first sample collected (e.g., an earlier longitudinal sample) relative to mutational frequency from cfDNA mutation analysis of a second sample (e.g., an later longitudinal sample) can be assessed based upon frequency of mutations (including de novo appearance) associated with regions targeted by tumor-naïve probe panels described herein.

To assess the effects of therapy on disease, the frequency of mutations (or alternatively estimated tumor genomes per ml of plasma) in cfDNA can be compared between a sample collected prior to therapy and a sample collected subsequent to therapy. An increase in mutational frequency from cfDNA mutation analysis of a sample collected prior to therapy relative to mutational frequency from cfDNA mutation analysis of a sample collected subsequent to therapy can be assessed as disease progression, unresponsiveness to therapy, and/or disease recurrence. A decrease in mutational frequency from cfDNA mutation analysis of a sample collected prior to therapy relative to mutational frequency from cfDNA mutation analysis of a sample collected subsequent to therapy can be assessed as a response. A response can be either a complete response (CR) or a partial response (PR). An increase in frequency of mutations in post-therapy cfDNA relative to pre-therapy cfDNA can indicate an increased likelihood that tumor burden of the subject is increasing. A decrease or maintenance of frequency of mutations in post-therapy cfDNA relative to pre-therapy cfDNA can indicate an increased likelihood that tumor burden of the subject is decreasing or stable.

Further therapy can be administered to a subject following an assessment step. For example, an initial measurement can be obtained from a patient before beginning a multi-dose anti-cancer therapy regimen. Subsequent measurements can be taken prior to administration of each dose. Analysis of variant-allele frequency in cfDNA at each stage can allow assessment of patient response to each dose of the therapy regimen. Assessment can further guide clinical decisions including dosages, therapy choices, etc. For example, clinical decisions (including introducing a new therapy or cessation of a current therapy) can be informed by assessment of tumor evasion mutation status associated with regions targeted by tumor-naïve probe panels described herein.

### Therapeutic Treatment

The methods described herein can follow the administration of a therapy to the patient. A therapy can comprise a cancer vaccine. A therapy can include targeted radiation therapy (e.g., external beam radiation, brachytherapy). A therapy can include an immune checkpoint inhibitor, including but not limited to a PD-1 inhibitor (e.g., nivolumab, pembrolizumab), a PD-L1 inhibitor (e.g., avelumab, durvalumab), or a CTLA-4 inhibitor (e.g., ipilimumab). A therapy can include targeted therapy technologies, such as monoclonal antibody therapies (e.g., trastuzumab, bevacizumab), retinoids (e.g., ATRA, bexarotene), selective steroid hormone receptor modulators (e.g., tamoxifen, toremifene), or inhibitors of oncoprotein such as tyrosine kinases (TK) (e.g., imatinib, erlotinib), mammalian target of rapamyciun (mTOR) (e.g., everolimus, temsirolimus), or histone deacetylase (HDAC) (e.g., valproate, vorinostat). A therapy can include cytotoxic chemotherapy. Examples of cytotoxic chemotherapeutic agents include cisplatin, carboplatin, oxaliplatin, nedaplatin, azacytidine, capecitabine, carmofur, cladribine, clofarabine, cytarabine, decitabine, florouracil, floxuridine, fludaramine, mercaptopurine, nelarabine, pentostatin, tegafur, tioguanine, methotrexate, pemetrexed, raltitrexed, hydroxycarbamide, irinotecan, topotecan, danorubicin, doxorubicin, epirubicin, idarubicin, mitoxantrone, valrubicin, etoposide, teniposide, docetaxel, paclitaxel, vinblastine, vincristine, vindesine, vinflunine, vinorelbine, bendamustine, busulfan, carmustine, chlorambucil, chlormethine, dacarbazine, fotemustine, ifosfamide, lomustine, melphalan, streptozotocin, gemcitabine, cyclophosphamide, temozolomide, dacarbazine, altretamine, bleomycin, bortezomib, actinomycin D, estramustine, ixabepilone, mitomycin, and procarbazine.

Also provided is a method of inducing a tumor specific immune response in a subject, vaccinating against a tumor, treating and/or alleviating a symptom of cancer in a subject by administering to the subject one or more antigens such as a plurality of antigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

An antigen can be administered in an amount sufficient to induce a CTL response. An antigen can be administered in an amount sufficient to induce a T cell response. An antigen can be administered in an amount sufficient to induce a B cell response.

An antigen can be administered alone or in combination with other therapeutic agents, e.g., a chemotherapeutic therapy, immune checkpoint blockade, and/or other immunotherapy.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, cancer, and/or subject-specific. For instance, the exact selection of peptides can be guided by expression patterns of the parent proteins in a given tissue or guided by mutation or disease status of a patient. The selection can be dependent on the specific type of cancer, the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for cancer, antigens with similar normal self-peptides that are expressed in high amounts in normal tissues can be avoided or be present in low amounts in a composition described herein. On the other hand, if it is known that the tumor of a patient expresses high amounts of a certain antigen, the respective pharmaceutical composition for treatment of a cancer can be present in high amounts and/or more than one antigen specific for this particularly antigen or pathway of this antigen can be included.

Compositions comprising an antigen can be administered to an individual already suffering from cancer. In therapeutic applications, compositions are administered to a patient in an amount sufficient to elicit a therapeutically effective response, e.g., in an amount sufficient to stimulate an effective CTL response to the tumor antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of an antigen, it is possible and can be felt desirable by the treating physician to administer substantial excesses of these compositions.

For therapeutic use, administration can begin at the detection or surgical removal of tumors. This can be followed by boosting doses until at least symptoms are substantially abated and for a period thereafter.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. Compositions can be administered at the site of surgical excision to induce a local immune response to the tumor. Compositions can be administered to target specific diseased tissues and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. Resulting aqueous solutions can be packaged for use as is, or lyophilized, the lyophilized preparation being combined with a sterile solution prior to administration. Compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to immune cells, a ligand to be incorporated into a liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, a nucleic acid can be delivered directly, as “naked DNA”. This approach is described, for instance, in Wolff et al., Science 247: 1465-1468 (1990) as well as U.S. Pat. Nos. 5,580,859 and 5,589,466. Nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

Nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. Sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, vector infected cells express the antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A vaccine can include an epitope-encoding nucleic acid whose sequence encodes one or more tumor and/or subject-specific mutations, such as one or more of the mutations whose frequency is determined in the cfDNA. A vaccine system can comprise a self-replicating alphavirus-based expression system encoding an epitope-encoding nucleic acid whose sequence encodes one or more tumor and/or subject-specific mutations. Self-replicating alphavirus-based expression systems for use as cancer vaccines are described in international patent application publication WO/2018/208856, which is herein incorporated by reference, in its entirety, for all purposes. A vaccine system can comprise a chimpanzee adenovirus (ChAdV)-based expression system encoding an epitope-encoding nucleic acid whose sequence encodes one or more tumor and/or subject-specific mutations. ChAdV-based expression system for use as cancer vaccines are described in international patent application publication WO/2018/098362, which is herein incorporated by reference, in its entirety, for all purposes.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art.

For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NSO cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to at least one nucleic acid sequence that encodes the antigen or vector. In certain embodiments the isolated polynucleotide can be cDNA.

### Antigens

Antigens can include nucleotides or polypeptides. For example, an antigen can be an RNA sequence that encodes for a polypeptide sequence. Antigens useful in vaccines can therefore include nucleotide sequences or polypeptide sequences. Antigens that can be used for cancer vaccines are described in international patent application publication WO/2019/226941, which is herein incorporated by reference, in its entirety, for all purposes.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be is immunogenic in a subject having a tumor, e.g., capable of eliciting a T cell response or a B cell response in the subject.

One or more antigens that induce an autoimmune response in a subject can be excluded from consideration in the context of vaccine generation for a subject having a tumor.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids-thus bypassing the need for computational or in vitro test-based selection of the strongest HLA-presented shorter peptide. In both cases, use of a longer peptide allows endogenous processing by patient cells and may lead to more effective antigen presentation and induction of T cell responses.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. The peptide contains the tumor specific mutation. In some aspects the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

The examples outline a cell-free DNA (cfDNA) assay used to monitor mutation frequency is provided. Additionally, data for on-treatment monitoring of mutation frequency in cfDNA from patient plasma was processed and analyzed using the provided protocol (see below) is presented. Notably, for GRANITE patients, greater than 200 mutations were monitored, representing all or a majority of high quality mutation calls associated with the tumor exome for each patient. The results demonstrate the method described provides a robust method for monitoring mutation frequency.

### Example 1—Cell-Free DNA Monitoring

### Methods

Below is a protocol for the methods describing the Cell-Free DNA Monitoring Assay.

### Plasma Sample Collection

Whole blood was drawn from patients at regularly scheduled clinical visits (approximately 1 month apart) that coincided with dosing. Whole blood was collected in 10 mL Streck cell-free DNA BCT tubes (Streck; La Vista, NE, USA) spun at 1600×g for 10 minutes at ambient temperature to separate the plasma layer, buffy coat, and red bloods. The plasma layer was removed and spun again at 5000×g for 10 minutes to remove any residual cellular material. The supernatant was collected and stored at −80° C. until extraction.

cfDNA Extraction and Quantification

Upon thawing separated plasma at ambient temperature, the plasma was spun at 5,000×g for 5 minutes to remove any cryoprecipitates formed during the storage process. cfDNA was extracted using the Apostle MiniMax cfDNA Isolation Kit (Beckman Coulter; Indianapolis, IN). Extracted cfDNA was quantified using the Qubit 1× High Sensitivity dsDNA Assay on a Qubit Fluorometer 4.0 (Thermo Fisher Scientific). For select samples, 1 uL was to visualize samples on an Agilent TapeStation using the HSD1000 kit.

gDNA isolation

For genomic DNA from each sample, 50,000 PMBCs were isolated and extracted using the Qiagen Tissue AllPrep Kit. For RNAlater samples, the Qiagen DNA/RNA Mini AllPrep kit was used to isolate genomic DNA from tissue that had been preserved in RNAlater.

### Library Preparation of Duplex Libraries and Hybrid Capture

Libraries were prepared with up to 20 ng cfDNA using the KAPA Hyper Prep kit per the manufacturer's instructions (KAPA Biosystems; Wilmington, MA). For libraries from gDNA, 30 ng of gDNA was first fragmented using the NEBNext Ultra II FS DNA Module (NEB, Ipswich, MA) with the following conditions: 25 minutes at 37° C. followed by 30 minutes at 65° C. After end repair, adaptor ligation was performed for 30 minutes with a pool of duplexed adaptors containing 5-mer non-random unique molecular identifiers (IDT, Coralville, Iowa). Whole library amplification was performed using the KAPA HiFi HotStart ReadyMix and NEBNext Multiple Oligos for Illumina (96 Unique Dual Index Primer Pairs).

After the preparation of duplex libraries, select regions of interest were hybridized to 750 ng duplex library overnight using custom-designed xGen Lockdown Probes and the xGen Hybridization and Wash kit per the manufacturer's instructions (IDT). Final libraries were quantified using the Qubit 1× High Sensitivity dsDNA assay and normalized.

### Sequencing and Analysis

Normalized samples were pooled in equimolar amounts and sequenced on a NovaSeq using a 2×151 bp and 8 bp index reads.

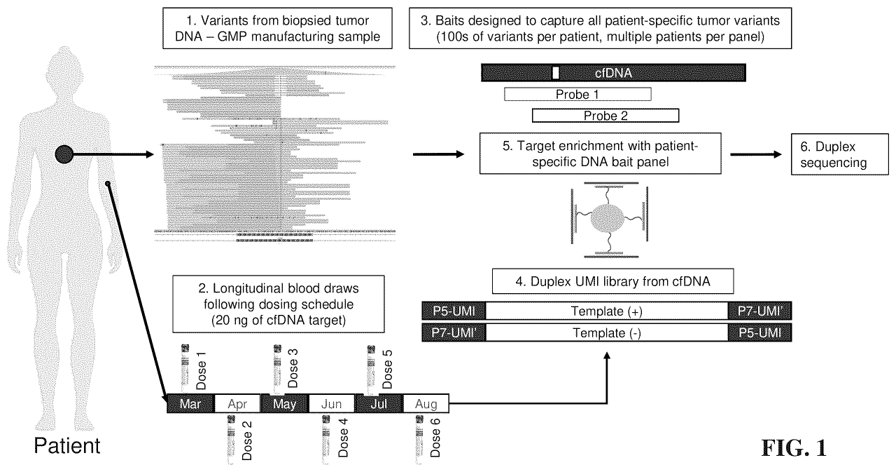

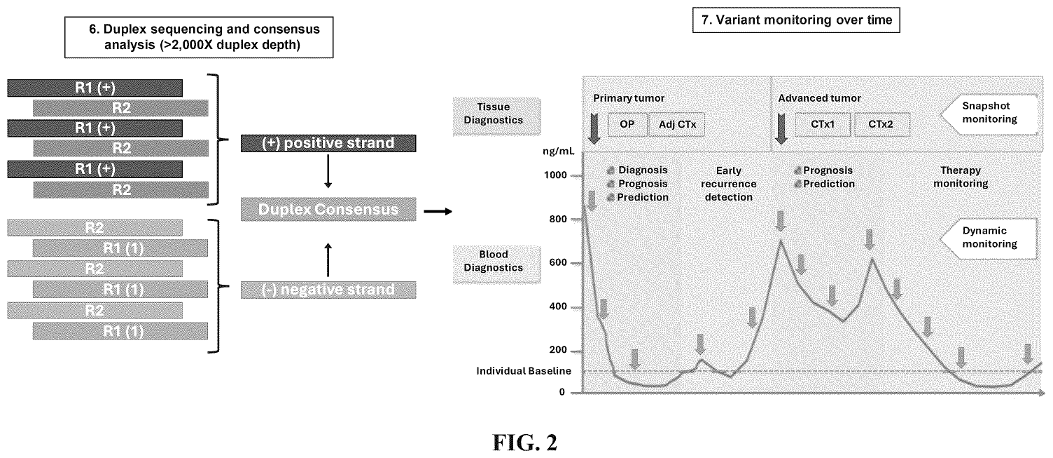

FIGS. 1 and 2 diagram and Table 1 shows the specifications for the process used to isolate and monitor mutant alleles in an individual patient's ctDNA.

TABLE 1
Assay Specifications for ctDNA Monitoring

| | |
|---|---|
| cfDNA input | 20 ng (as low as 5 ng - limited variant sensitivity) |
| Panel footprint | 296 Kb |
| Number of baits | 5,460 |
| Variants per patient | 90-461 (mean: 283) |
| Duplex depth | 2000X-4000X duplex consensus |

Tumor-specific DNA variant alleles were identified in patients from biopsied tumor tissue and used to create baits to isolate tumor-specific DNA from all circulating cell-free DNA (cfDNA) in patient blood samples. Isolated ctDNA was duplex sequenced and analyzed for duplex consensus. Sequencing of multiple blood draws over the course of treatment allowed less-invasive monitoring of patient response.

After sequencing was done and FASTQ files generated, the UMI was extracted and assigned to each read tag before alignment with BWA-MEM (Durbin et al, Bioinformatics, 2010). fgbio toolkit (Fulcrum Genomics) was used to group reads by UMI on aligned bam files and call Duplex consensus reads. Prior to data analysis, the unaligned bam files from fgbio were aligned using BWA-MEM and then used freebayes (Marth et al, arXiv 2012) to get Variant Allele Frequency (VAF) of each somatic variant of interest.

### Cancer Vaccine Administration

An open-label, multi-center, multi-dose Phase 1/2 study was performed to assess the dose, safety and tolerability, immunogenicity, and early clinical activity of a heterologous prime/boost vaccination strategy. Two vaccine programs, GRANITE and SLATE, were assessed. The clinical trial design is described in international patent application publication WO/2019/226941, which is herein incorporated by reference, in its entirety, for all purposes.

A personalized neoantigen cancer vaccine (“GRANITE”) was administered in combination with immune checkpoint blockade in patients with advanced cancer. The GRANITE heterologous prime/boost vaccine regimen included (1) a ChAdV that is used as a prime vaccination [GRT-C901] and (2) a SAM formulated in a LNP that is used for boost vaccinations [GRT-R902] following GRT-C901. The ChAdV vector is based on a modified ChAdV68 sequence. The SAM vector is based on an RNA alphavirus backbone. Both GRT-C901 and GRT-R902 expressed the same 20 personalized neoantigens as well as two universal CD4 T-cell epitopes (PADRE and Tetanus Toxoid). Tumors were used for whole-exome and transcriptome sequencing to detect somatic mutations, and blood was used for HLA typing and detection/subtraction of germline exome variants to generate the personalized neoantigen cassette using the EDGE algorithm for 10 subjects (Patients 1-10, referred to herein as patients G1-G10).

A shared neoantigen cancer vaccine (“SLATE”) was administered in combination with immune checkpoint blockade in patients with advanced cancer. The SLATE heterologous prime/boost vaccine regimen included (1) a ChAdV that is used as a prime vaccination [GRT-C903] and (2) a SAM formulated in a LNP that is used for boost vaccinations [GRT-R904] following GRT-C903. Both GRT-C903 and GRT-R904 expressed the same 20 shared neoantigens derived from a specific list of oncogenic mutations as well as two universal CD4 T-cell epitopes (PADRE and Tetanus Toxoid). For subject inclusion, tumors were used for whole-exome and transcriptome sequencing to detect somatic mutations, and blood was used for HLA typing. Enrolled SLATE subjects were determined to have HLA A02:01 and KRAS mutation G12C predicted to be presented by HLA A02:01 (Patients S1, S2, and S3), HLA A01:01 and KRAS mutation Q61H predicted to be presented by HLA A01:01 (Patients S4 and S7), or HLA A03:01 or A11:01 and KRAS mutation G12V predicted to be presented by HLA A03:01 or A11:01 (A03:01 for Patient S9; A11:01 for Patients S11 and S15).

Both treatment studies (i.e., the GRANITE and SLATE vaccine regimens) administered the vaccine via IM injection bilaterally (e.g., in each deltoid muscle) in combination with immune checkpoint blockade, specifically SC ipilimumab and IV nivolumab. The studies followed two sequential phases.

GRT-C901 and GRT-C903 are replication-defective, E1 and E3 deleted adenoviral vectors based on chimpanzee adenovirus 68. The vector contained an expression cassette encoding 20 neoantigens as well as two universal CD4 T-cell epitopes (PADRE and Tetanus Toxoid). GRT-C901 and GRT-C903 were formulated in solution at 5×10¹¹ vp/mL and 1.0 mL was injected IM at each of 2 bilateral vaccine injection sites in opposing deltoid muscles. The GRT-C901 and GRT-C903 vectors differ only by the encoded neoantigens within the cassette.

GRT-R902 and GRT-R904 are SAM vectors derived from an alphavirus. The GRT-R902 and GRT-R904 vectors encoded the viral proteins and the 5′ and 3′ RNA sequences required for RNA amplification but encoded no structural proteins. The SAM vectors were formulated in LNPs that included 4 lipids: an ionizable amino lipid, a phosphatidylcholine, cholesterol, and a PEG-based coat lipid to encapsulate the SAM and form LNPs. The GRT-R902 vector contained the same neoantigen expression cassette as used in GRT-C901 for each patient, respectively. The GRT-R904 vector contained the same neoantigen expression cassette as used in GRT-C903. GRT-R902 and GRT-R904 were formulated in solution at 1 mg/mL and was injected IM at each of 2 bilateral vaccine injection sites in opposing deltoid muscles (deltoid muscle preferred, gluteus [dorso or ventro] or rectus femoris on each side may be used). The boost vaccination sites were as close to the prime vaccination site as possible. The injection volume was based on the dose to be administered. The dose level amount refers explicitly to the amount of the SAM vector, i.e., it does not refer to other components, such as the LNP. The ratio of LNP:SAM was approximately 24:1. Accordingly, the dose of LNP was 720 μg, 2400 μg, and 7200 μg for each respective GRT-R902/GRT-R904 dose level (see below).

Ipilimumab is a human monoclonal IgG1 antibody that binds to the cytotoxic T-lymphocyte associated antigen 4 (CTLA-4). Ipilimumab was formulated in solution at 5 mg/mL and was injected SC proximally (within −2 cm) to each of the bilateral vaccination sites. Ipilimumab was administered at a dose of 30 mg of antibody in four 1.5 mL (7.5 mg) injections proximal to the vaccine draining LN at each of the bilateral vaccination sites (i.e., 1.5 mL below the vaccination site and 1.5 mL above the vaccination site on each bilateral side in each deltoid, ventrogluteal, dorsogluteal, or rectus femoris [deltoid preferred, but dependent on clinical site and patient preference])

Nivolumab is a human monoclonal IgG4 antibody that blocks the interaction of PD-1 and its ligands, PD-L1 and PD-L2. Nivolumab was formulated in solution at 10 mg/mL and was administered as an IV infusion (480 mg) through a 0.2-micron to 1.2-micron pore size, low-protein binding in-line filter at the protocol-specified doses. It was not administered as an IV push or bolus injection. Nivolumab infusion was promptly followed by a flush of diluent to clear the line. Nivolumab was administered following each vaccination (i.e., each of GRT-C901, GRT-R902, GRT-C903, or GRT-R904) with or without ipilimumab on the same day. The dose and route of nivolumab was based on the Food and Drug Administration approved dose and route.

### Results

Monitoring of ctDNA in cfDNA-containing samples was used to track patient response to therapy. Specifically, patients receiving tumor neoantigen-based vaccine therapies (GRANITE and SLATE) were monitored over the course of treatment. Sequencing of cancer exome associated mutations was conducted at both high target coverage and at high read depth.

The ctDNA of two separate patients (G1 and G2) receiving GRANITE therapy were monitored to examine response. The details of all ctDNA isolations from each patient are given in Table 2.

TABLE 2
Details of ctDNA Isolations from Patients
Receiving GRANITE Therapy

| | | Yield | Total | Plasma | |
|---|---|---|---|---|---|
| Patient | Sample | ng/uL | ng | mL | ng/mL |
| G1 | Dose 1 Day 1 | 0.526 | 52.6 | 6.00 | 8.77 |
| (Pt0009) | Dose 2 Day 1 | 0.388 | 38.8 | 7.75 | 5.01 |
| | Dose 3 Day 1 | 0.471 | 47.1 | 8.00 | 5.89 |
| | Dose 4 Day 1 | 0.461 | 34.6 | 5.25 | 6.59 |
| | Dose 5 Day 1 | 0.407 | 20.4 | 3.25 | 6.26 |
| | Dose 6 Day 1 | 0.248 | 12.4 | 2.75 | 4.51 |
| | Dose 7 Day 1 | 0.766 | 22.98 | 2.50 | 9.19 |
| | Dose 8 Day 1 | 2.00 | 100 | 5.75 | 17.39 |
| G2 | Dose 1 Day 1 | 1.34 | 107.2 | 8.00 | 13.40 |
| (Pt0005) | Dose 2 Day 1 | 2.19 | 219.0 | 10.0 | 23.05 |
| | Dose 3 Day 1A | 2.92 | 262.8 | 9.00 | 31.85 |
| | Dose 3 Day 1B | 2.43 | 218.7 | 6.75 | 32.40 |
| | Dose 4 Day 1 | 3.89 | 291.8 | 7.00 | 41.68 |
| | Dose 5 Day 1 | 1.38 | 103.5 | 8.25 | 12.55 |

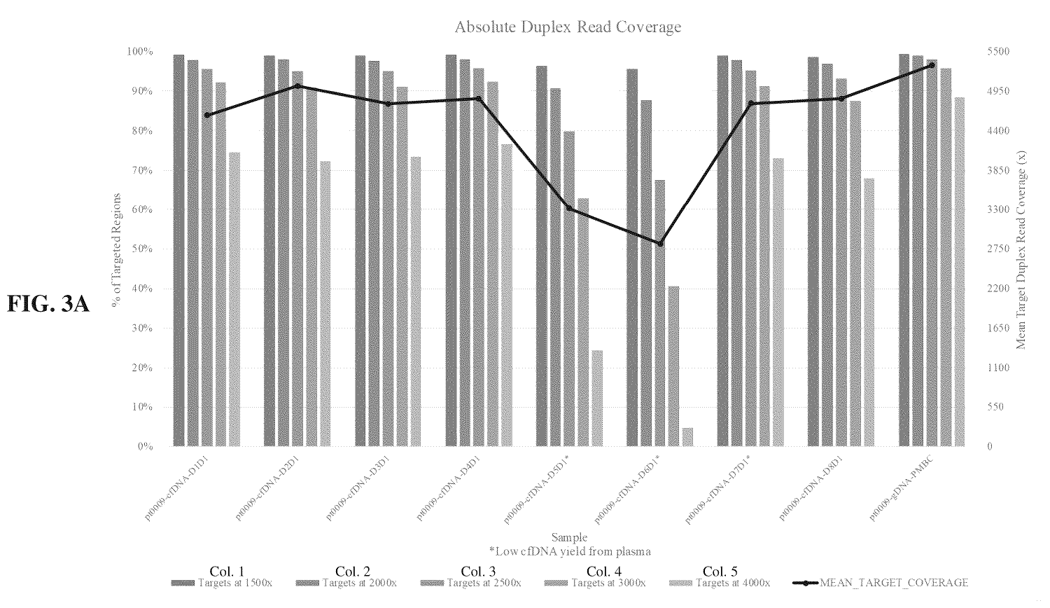

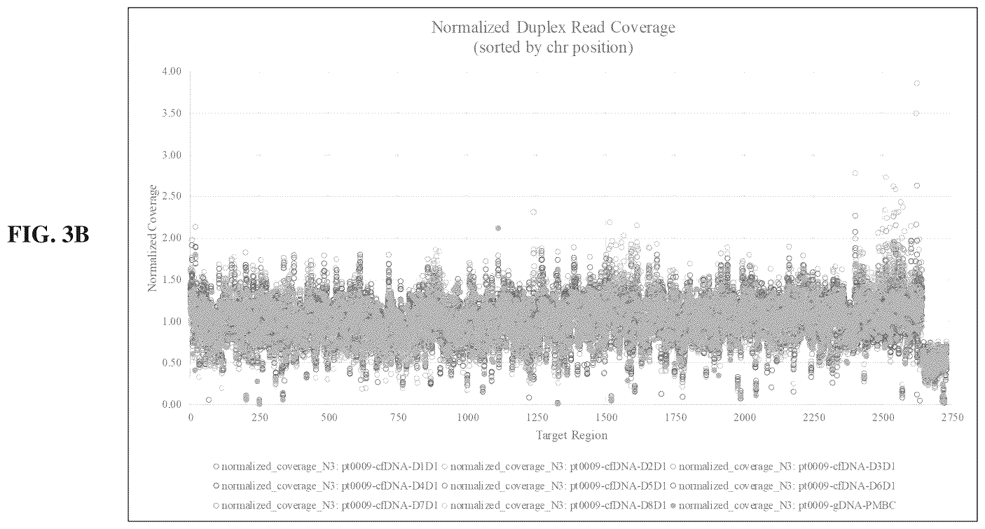

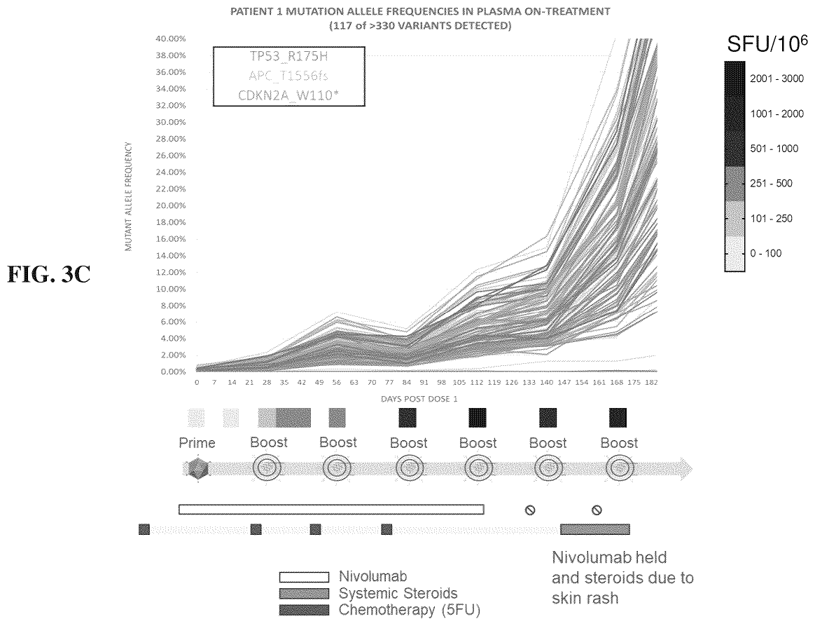

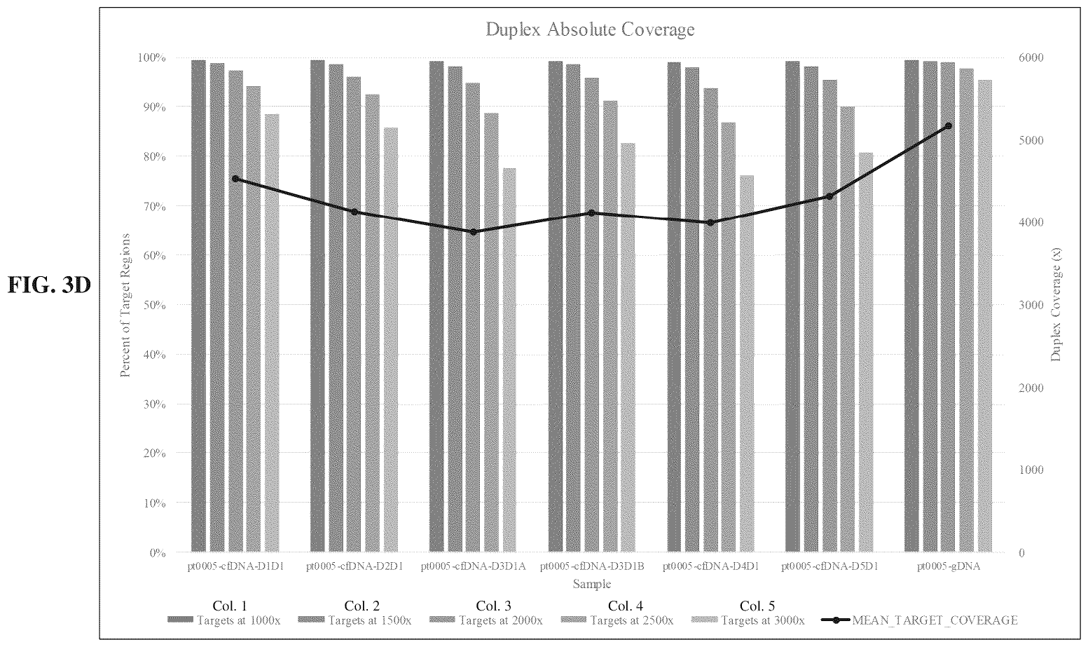

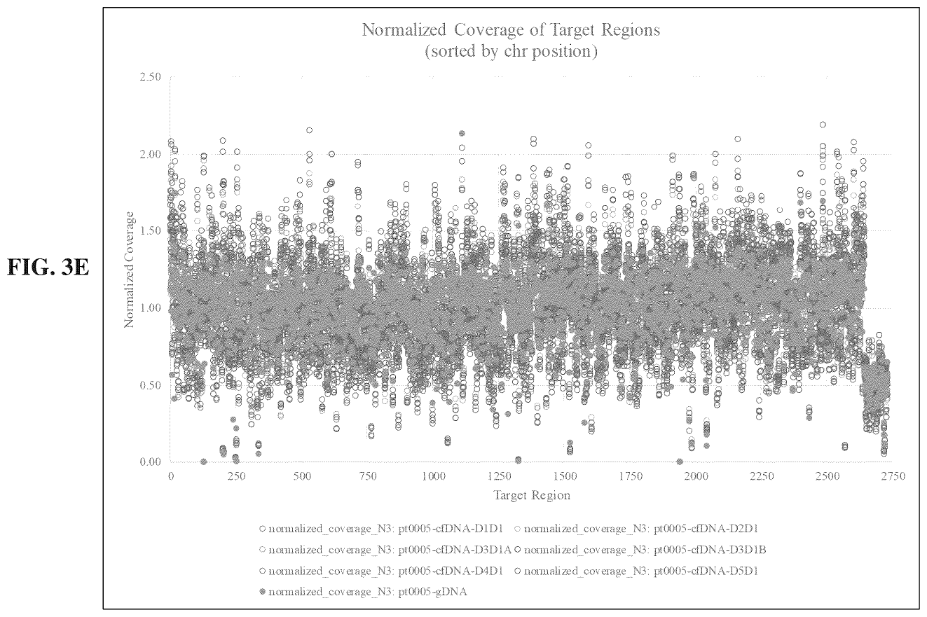

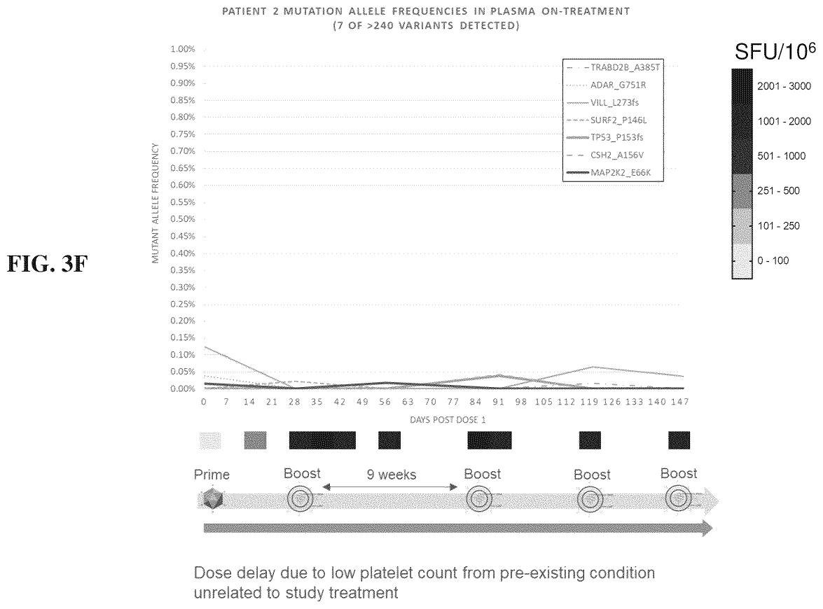

Duplex read coverage over the course of treatment for patient G1 is shown in FIG. 3A and FIG. 3B. Mean sequencing read depth (mean target duplex read coverage [x]) for targets ranged from 2817×-5017× in cfDNA samples with >87% of targets (greater than 330 variants monitored) with >2000× duplex reads and >68% of targets with >4000× duplex read (excluding D5D1 and D6D1). The sequencing profile demonstrated high target coverage at high read depth.

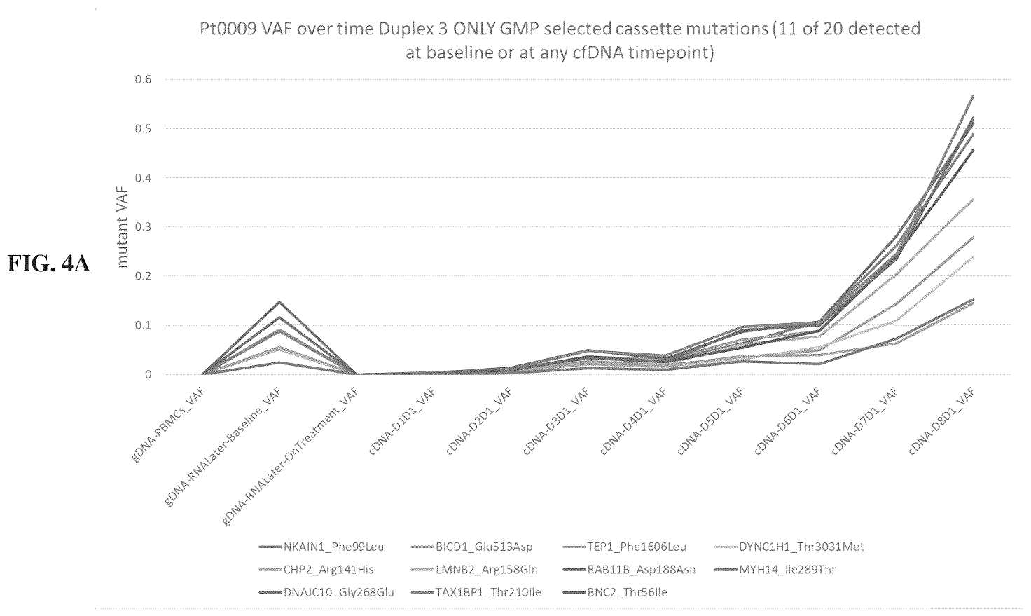

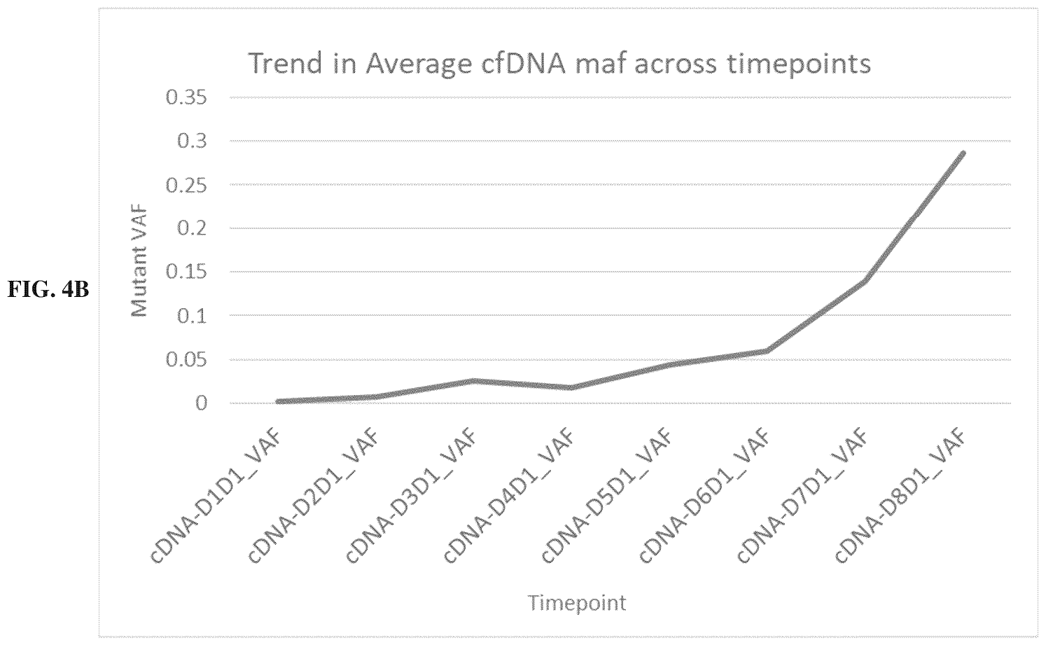

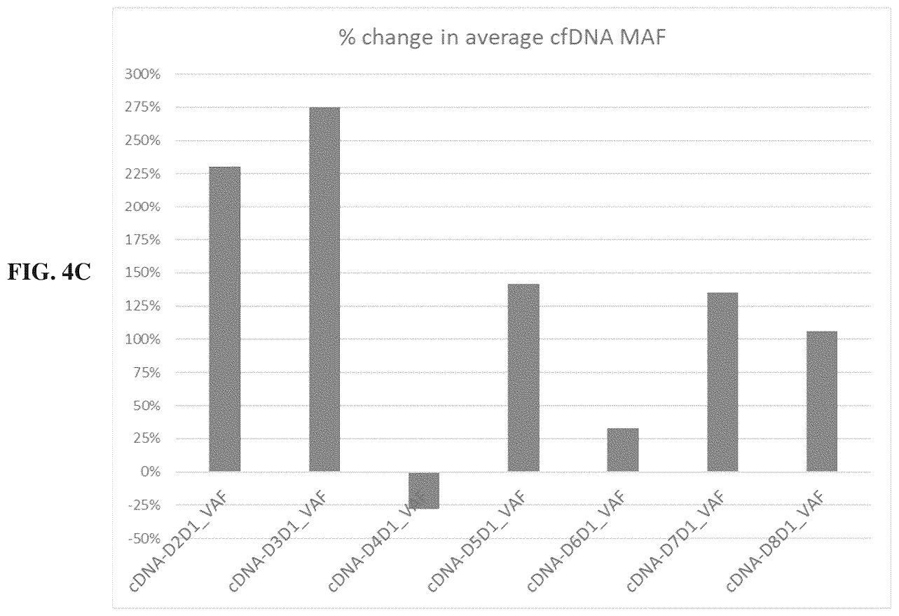

Mutation allele frequency in cfDNA was monitored over the course of treatment for GRANITE patient G1. As shown in FIG. 3C and Table 3, 117 mutant alleles out of greater than 330 subject and tumor-specific variants were monitored in the ctDNA of G1. FIG. 4A-C also shows the frequency of mutant alleles in ctDNA isolated from G1 over the course of disease. FIG. 4A shows mutant allele frequency for 11 of 20 mutations detected at baseline. FIG. 4B shows average mutant allele frequency. FIG. 4C shows the percent change in the average mutant allele frequency. An initial spike in tumor-specific variant allele frequency (VAF), which is also given as mutant allele frequency (MAF), following doses 1 and 2 is followed by a decrease after dose 3 suggesting a response to treatment, then increased moderately over the first 168 days, correlating with stable disease. Mutant allele frequency then noticeably increased after day 168 (week 24), correlating with progressive disease. Accordingly, monitoring mutation allele frequency in cfDNA served as an effective non-invasive proxy for monitoring status of disease, including assessing disease progression and the efficacy of a therapeutic regimen.

TABLE 3
VAF Values from c(DNA of Patient G1

| | | gDNA- | gDNA- | | | | | | | | |
|---|---|---|---|---|---|---|---|---|---|---|---|
| | gDNA- | RNALater- | RNALater- | cfDNA- | cfDNA- | cfDNA- | cfDNA- | cfDNA- | cfDNA- | cfDNA- | cfDNA- |
| | PBMCs | Baseline | OnTreatment | D1D1 | D2D1 | D3D1 | D4D1 | D5D1 | D6D1 | D7D1 | D8D1 |
| Variants | VAF | VAF | VAF | VAF | VAF | VAF | VAF | VAF | VAF | VAF | VAF |
| NKAIN1_Phe99Leu | 0.000 | 0.090 | 0.000 | 0.002 | 0.007 | 0.036 | 0.027 | 0.063 | 0.108 | 0.263 | 0.489 |
| TXLNA_Glul161Lys | 0.000 | 0.075 | 0.000 | 0.004 | 0.007 | 0.032 | 0.016 | 0.048 | 0.086 | 0.190 | 0.417 |
| FPGT_Pro141His | 0.000 | 0.079 | 0.000 | 0.001 | 0.006 | 0.027 | 0.020 | 0.051 | 0.080 | 0.164 | 0.431 |
| FLG_Glu1962Lys | 0.000 | 0.070 | 0.000 | 0.001 | 0.005 | 0.029 | 0.021 | 0.049 | 0.087 | 0.171 | 0.334 |
| KIRREL_Gly160Trp | 0.000 | 0.071 | 0.000 | 0.003 | 0.007 | 0.028 | 0.020 | 0.060 | 0.082 | 0.189 | 0.345 |
| INFSF4_His46Tyr | 0.000 | 0.077 | 0.000 | 0.001 | 0.008 | 0.018 | 0.021 | 0.038 | 0.050 | 0.128 | 0.232 |
| RC3H1_Pro409Ala | 0.000 | 0.069 | 0.000 | 0.002 | 0.012 | 0.029 | 0.028 | 0.050 | 0.068 | 0.156 | 0.359 |
| XPR1_Arg157Gln | 0.000 | 0.093 | 0.000 | 0.003 | 0.013 | 0.045 | 0.034 | 0.077 | 0.099 | 0.189 | 0.400 |
| RGS18_Glu159* | 0.000 | 0.074 | 0.000 | 0.002 | 0.005 | 0.022 | 0.015 | 0.035 | 0.052 | 0.127 | 0.244 |
| MALRD1_Asp1770Tyr | 0.000 | 0.062 | 0.000 | 0.002 | 0.008 | 0.020 | 0.018 | 0.036 | 0.048 | 0.119 | 0.205 |
| PRKG1_Glu472Lys | 0.000 | 0.049 | 0.000 | 0.001 | 0.005 | 0.014 | 0.013 | 0.027 | 0.033 | 0.042 | 0.110 |
| TMEM26_Thr103Asn | 0.000 | 0.098 | 0.000 | 0.004 | 0.012 | 0.043 | 0.038 | 0.070 | 0.086 | 0.171 | 0.377 |
| GRID1_Asp670Glu | 0.000 | 0.052 | 0.000 | 0.001 | 0.006 | 0.021 | 0.011 | 0.037 | 0.052 | 0.110 | 0.238 |
| SORCS3_Ala4Val | 0.000 | 0.055 | 0.000 | 0.000 | 0.003 | 0.020 | 0.017 | 0.044 | 0.044 | 0.116 | 0.262 |
| C2CD3_Ser2088Phe | 0.000 | 0.024 | 0.000 | 0.000 | 0.001 | 0.003 | 0.002 | 0.004 | 0.013 | 0.013 | 0.023 |
| PICALM_Lys40Arg | 0.000 | 0.109 | 0.000 | 0.005 | 0.023 | 0.072 | 0.051 | 0.123 | 0.150 | 0.405 | 0.709 |
| C2CD2L_Thr673Met | 0.000 | 0.000 | 0.000 | 0.000 | 0.000 | 0.000 | 0.000 | 0.000 | 0.000 | 0.000 | 0.000 |
| BICD1_Glu513Asp | 0.000 | 0.053 | 0.000 | 0.001 | 0.008 | 0.027 | 0.019 | 0.036 | 0.049 | 0.144 | 0.279 |
| COL2A1_Arg989His | 0.000 | 0.000 | 0.000 | 0.000 | 0.000 | 0.000 | 0.000 | 0.000 | 0.000 | 0.000 | 0.000 |
| SCN8A_Arg558Cys | 0.000 | 0.051 | 0.000 | 0.004 | 0.008 | 0.026 | 0.023 | 0.050 | 0.071 | 0.158 | 0.306 |
| C12orf80_Thr102fs | 0.000 | 0.053 | 0.000 | 0.001 | 0.006 | 0.019 | 0.016 | 0.030 | 0.051 | 0.129 | 0.267 |
| LRP1_Glu3658Val | 0.000 | 0.064 | 0.000 | 0.002 | 0.004 | 0.027 | 0.017 | 0.041 | 0.044 | 0.141 | 0.275 |
| LRIG3_Gln750* | 0.000 | 0.079 | 0.000 | 0.001 | 0.005 | 0.020 | 0.014 | 0.033 | 0.041 | 0.112 | 0.263 |
| SETD1B_Arg323Cys | 0.000 | 0.061 | 0.000 | 0.001 | 0.004 | 0.027 | 0.017 | 0.045 | 0.058 | 0.125 | 0.275 |
| AMER2_Glu336Lys | 0.000 | 0.093 | 0.000 | 0.005 | 0.011 | 0.038 | 0.024 | 0.074 | 0.078 | 0.155 | 0.357 |
| SPG20_Thr84Ala | 0.000 | 0.000 | 0.000 | 0.000 | 0.000 | 0.000 | 0.001 | 0.002 | 0.000 | 0.001 | 0.003 |
| TEP1_Phe1606Leu | 0.000 | 0.051 | 0.000 | 0.005 | 0.009 | 0.036 | 0.021 | 0.061 | 0.078 | 0.204 | 0.356 |
| BAZ1A_Pro610Pro | 0.000 | 0.000 | 0.000 | 0.000 | 0.000 | 0.000 | 0.000 | 0.000 | 0.000 | 0.001 | 0.000 |
| NID2_Arg497Trp | 0.000 | 0.000 | 0.000 | 0.000 | 0.000 | 0.000 | 0.000 | 0.000 | 0.000 | 0.000 | 0.001 |
| ESRRB_Thr389Met | 0.000 | 0.058 | 0.000 | 0.002 | 0.006 | 0.031 | 0.021 | 0.047 | 0.068 | 0.148 | 0.265 |
| FLRT2_Pro419Ser | 0.000 | 0.041 | 0.000 | 0.000 | 0.007 | 0.031 | 0.016 | 0.043 | 0.060 | 0.171 | 0.320 |
| SERPINA12_Ser182Asn | 0.000 | 0.000 | 0.000 | 0.000 | 0.000 | 0.000 | 0.000 | 0.000 | 0.001 | 0.000 | 0.000 |
| DYNC1H1_Thr3031Met | 0.000 | 0.054 | 0.000 | 0.003 | 0.006 | 0.021 | 0.015 | 0.031 | 0.056 | 0.109 | 0.239 |
| OTUD7A_Gly286Gly | 0.000 | 0.077 | 0.000 | 0.006 | 0.012 | 0.052 | 0.033 | 0.083 | 0.110 | 0.273 | 0.570 |
| RYR3_Gln241His | 0.000 | 0.080 | 0.000 | 0.005 | 0.013 | 0.053 | 0.038 | 0.099 | 0.114 | 0.269 | 0.556 |
| SEMA6D_Gln651* | 0.000 | 0.057 | 0.000 | 0.004 | 0.010 | 0.038 | 0.024 | 0.072 | 0.091 | 0.219 | 0.475 |
| TICRR_Arg931Gln | 0.000 | 0.000 | 0.000 | 0.000 | 0.000 | 0.000 | 0.000 | 0.000 | 0.000 | 0.000 | 0.000 |
| LMF1_Ala469Thr | 0.000 | 0.100 | 0.000 | 0.004 | 0.013 | 0.040 | 0.031 | 0.079 | 0.108 | 0.265 | 0.558 |
| ZNF598_Glu777Lys | 0.000 | 0.110 | 0.001 | 0.002 | 0.010 | 0.034 | 0.034 | 0.067 | 0.102 | 0.311 | 0.568 |
| CHP2_Arg141His | 0.000 | 0.091 | 0.000 | 0.002 | 0.008 | 0.037 | 0.030 | 0.071 | 0.088 | 0.241 | 0.517 |
| NFATC2IP_Asp196Glu | 0.000 | 0.071 | 0.000 | 0.004 | 0.007 | 0.036 | 0.019 | 0.048 | 0.085 | 0.201 | 0.449 |
| WWP2_Thr483Met | 0.000 | 0.001 | 0.000 | 0.000 | 0.000 | 0.000 | 0.000 | 0.000 | 0.000 | 0.000 | 0.000 |
| DDX19B_Arg11His | 0.000 | 0.100 | 0.000 | 0.004 | 0.016 | 0.061 | 0.039 | 0.102 | 0.125 | 0.333 | 0.641 |
| MYO1C_Gly11Arg | 0.000 | 0.001 | 0.000 | 0.000 | 0.000 | 0.000 | 0.000 | 0.000 | 0.000 | 0.000 | 0.000 |
| TP53_Arg175His | 0.000 | 0.117 | 0.000 | 0.003 | 0.013 | 0.045 | 0.036 | 0.079 | 0.105 | 0.245 | 0.555 |
| HNF1B_Arg310Trp | 0.000 | 0.103 | 0.001 | 0.002 | 0.011 | 0.038 | 0.024 | 0.059 | 0.086 | 0.191 | 0.392 |
| SMARCE1_Thr361Met | 0.000 | 0.050 | 0.000 | 0.001 | 0.005 | 0.016 | 0.009 | 0.028 | 0.035 | 0.080 | 0.170 |
| BZRAP1_Leu256Phe | 0.000 | 0.049 | 0.000 | 0.001 | 0.006 | 0.019 | 0.015 | 0.025 | 0.035 | 0.081 | 0.177 |
| MEP1B_Arg622* | 0.000 | 0.046 | 0.001 | 0.001 | 0.005 | 0.011 | 0.013 | 0.018 | 0.035 | 0.070 | 0.144 |
| SETBP1_Lys675Arg | 0.000 | 0.050 | 0.000 | 0.002 | 0.005 | 0.013 | 0.018 | 0.032 | 0.044 | 0.075 | 0.183 |
| LOXHD1_Arg915Gln | 0.000 | 0.054 | 0.000 | 0.003 | 0.008 | 0.045 | 0.023 | 0.058 | 0.105 | 0.232 | 0.450 |
| SMAD4_Arg497His | 0.000 | 0.044 | 0.000 | 0.002 | 0.006 | 0.019 | 0.010 | 0.035 | 0.037 | 0.084 | 0.169 |
| DCC_Val50Ile | 0.000 | 0.102 | 0.000 | 0.008 | 0.017 | 0.062 | 0.048 | 0.112 | 0.163 | 0.338 | 0.635 |
| LMNB2_Arg158Gln | 0.000 | 0.056 | 0.000 | 0.001 | 0.005 | 0.021 | 0.017 | 0.038 | 0.039 | 0.063 | 0.145 |
| RAB11B_Asp188Asn | 0.000 | 0.088 | 0.000 | 0.002 | 0.008 | 0.036 | 0.026 | 0.055 | 0.089 | 0.242 | 0.457 |
| ZNF414_Arg337Trp | 0.000 | 0.037 | 0.000 | 0.001 | 0.005 | 0.021 | 0.013 | 0.031 | 0.037 | 0.064 | 0.139 |
| ZNF878_Pro170fs | 0.000 | 0.000 | 0.000 | 0.000 | 0.000 | 0.000 | 0.000 | 0.000 | 0.000 | 0.000 | 0.000 |
| ZNF729_Asn498Lys | 0.000 | 0.052 | 0.000 | 0.002 | 0.004 | 0.021 | 0.015 | 0.039 | 0.039 | 0.054 | 0.138 |
| RINL_Asp27Tyr | 0.000 | 0.026 | 0.000 | 0.002 | 0.003 | 0.018 | 0.013 | 0.031 | 0.035 | 0.043 | 0.087 |
| FCGBP_Arg154Lys | 0.000 | 0.042 | 0.000 | 0.001 | 0.004 | 0.014 | 0.010 | 0.022 | 0.033 | 0.070 | 0.112 |
| PVR_Arg269His | 0.000 | 0.060 | 0.000 | 0.002 | 0.008 | 0.032 | 0.023 | 0.056 | 0.065 | 0.163 | 0.318 |
| RTN2_Arg78Cys | 0.000 | 0.160 | 0.000 | 0.005 | 0.016 | 0.059 | 0.040 | 0.115 | 0.145 | 0.304 | 0.547 |
| MYH14_Ile289Thr | 0.000 | 0.147 | 0.000 | 0.003 | 0.013 | 0.049 | 0.033 | 0.086 | 0.108 | 0.282 | 0.511 |
| ZNF160_Ser487Ile | 0.000 | 0.128 | 0.000 | 0.003 | 0.016 | 0.066 | 0.037 | 0.105 | 0.127 | 0.296 | 0.471 |
| ZNF581_Pro24Ala | 0.000 | 0.039 | 0.000 | 0.002 | 0.004 | 0.013 | 0.007 | 0.019 | 0.027 | 0.065 | 0.096 |
| PEG3_Pro612Leu | 0.000 | 0.038 | 0.000 | 0.002 | 0.006 | 0.018 | 0.015 | 0.030 | 0.032 | 0.048 | 0.093 |
| MYT1L_Gly557Arg | 0.000 | 0.000 | 0.000 | 0.000 | 0.000 | 0.000 | 0.000 | 0.000 | 0.000 | 0.000 | 0.000 |
| SMC6_Leu384Phe | 0.000 | 0.071 | 0.000 | 0.003 | 0.011 | 0.027 | 0.021 | 0.060 | 0.063 | 0.189 | 0.409 |
| OTOF_Asp1579Asn | 0.000 | 0.021 | 0.000 | 0.002 | 0.004 | 0.015 | 0.010 | 0.022 | 0.038 | 0.100 | 0.195 |
| NRXN1_Asp866Tyr | 0.000 | 0.030 | 0.000 | 0.001 | 0.004 | 0.013 | 0.007 | 0.031 | 0.037 | 0.095 | 0.173 |
| CTNNA2_Thr574Lys | 0.000 | 0.064 | 0.000 | 0.006 | 0.010 | 0.035 | 0.026 | 0.059 | 0.078 | 0.178 | 0.330 |
| KDM3A_Val610Ile | 0.000 | 0.078 | 0.000 | 0.002 | 0.010 | 0.037 | 0.021 | 0.048 | 0.058 | 0.160 | 0.341 |
| SH3RF3_Glu91Lys | 0.000 | 0.090 | 0.000 | 0.003 | 0.010 | 0.041 | 0.021 | 0.057 | 0.069 | 0.172 | 0.350 |
| EN1_Arg284His | 0.000 | 0.059 | 0.000 | 0.002 | 0.007 | 0.028 | 0.024 | 0.065 | 0.089 | 0.182 | 0.306 |
| PTPN4_Lys318Asn | 0.000 | 0.076 | 0.000 | 0.002 | 0.011 | 0.025 | 0.026 | 0.052 | 0.057 | 0.137 | 0.313 |
| LRP1B_Ala3706Ser | 0.000 | 0.047 | 0.000 | 0.003 | 0.008 | 0.022 | 0.025 | 0.039 | 0.073 | 0.137 | 0.268 |
| TTN_Val18074Phe | 0.000 | 0.055 | 0.000 | 0.002 | 0.006 | 0.027 | 0.023 | 0.056 | 0.074 | 0.171 | 0.290 |
| DNAJC10_Gly268Glu | 0.000 | 0.025 | 0.000 | 0.000 | 0.003 | 0.013 | 0.009 | 0.027 | 0.021 | 0.073 | 0.153 |
| ACSS1_Ala617Thr | 0.000 | 0.045 | 0.000 | 0.002 | 0.003 | 0.020 | 0.015 | 0.026 | 0.036 | 0.080 | 0.116 |
| TUBGCP6_Thr1792Ile | 0.000 | 0.052 | 0.000 | 0.002 | 0.006 | 0.032 | 0.023 | 0.045 | 0.070 | 0.188 | 0.431 |
| CNTN4_Asp407His | 0.000 | 0.024 | 0.000 | 0.002 | 0.003 | 0.011 | 0.011 | 0.024 | 0.040 | 0.087 | 0.203 |
| BSN_Ala329Val | 0.000 | 0.056 | 0.000 | 0.001 | 0.007 | 0.023 | 0.019 | 0.040 | 0.059 | 0.146 | 0.324 |
| TMEM108_Leu19fs | 0.000 | 0.053 | 0.000 | 0.002 | 0.005 | 0.028 | 0.018 | 0.048 | 0.070 | 0.150 | 0.316 |
| ZIC1_Gln177Lys | 0.000 | 0.037 | 0.000 | 0.001 | 0.001 | 0.014 | 0.011 | 0.019 | 0.034 | 0.094 | 0.207 |
| AHSG_Val102Leu | 0.000 | 0.028 | 0.000 | 0.001 | 0.005 | 0.029 | 0.017 | 0.037 | 0.050 | 0.147 | 0.270 |
| MASP1_Ser445Asn | 0.000 | 0.013 | 0.000 | 0.001 | 0.006 | 0.020 | 0.015 | 0.029 | 0.037 | 0.116 | 0.249 |
| IL1RAP_Arg576His | 0.000 | 0.029 | 0.000 | 0.002 | 0.006 | 0.016 | 0.013 | 0.028 | 0.036 | 0.114 | 0.235 |
| TFRC_Phe621Ile | 0.000 | 0.026 | 0.000 | 0.002 | 0.004 | 0.020 | 0.011 | 0.031 | 0.043 | 0.127 | 0.235 |
| GUF1_Leu254Leu | 0.000 | 0.048 | 0.000 | 0.003 | 0.004 | 0.026 | 0.020 | 0.032 | 0.062 | 0.104 | 0.273 |
| THEGL_Ala337Val | 0.000 | 0.023 | 0.000 | 0.002 | 0.005 | 0.009 | 0.007 | 0.021 | 0.033 | 0.085 | 0.207 |
| SLC9B1_Glu53Gly | 0.000 | 0.077 | 0.001 | 0.001 | 0.010 | 0.029 | 0.028 | 0.061 | 0.086 | 0.203 | 0.479 |
| MSH3_Thr282Ile | 0.000 | 0.000 | 0.000 | 0.000 | 0.000 | 0.000 | 0.000 | 0.000 | 0.000 | 0.002 | 0.000 |
| ADGRV1_Ala1254Glu | 0.000 | 0.083 | 0.000 | 0.002 | 0.008 | 0.027 | 0.023 | 0.063 | 0.059 | 0.150 | 0.409 |
| APC_Thr1556fs | 0.000 | 0.082 | 0.000 | 0.004 | 0.010 | 0.042 | 0.024 | 0.082 | 0.090 | 0.205 | 0.450 |
| MCC_Gly153Ala | 0.000 | 0.078 | 0.000 | 0.004 | 0.011 | 0.041 | 0.026 | 0.060 | 0.078 | 0.219 | 0.473 |
| FBN2_Glu1596Lys | 0.000 | 0.079 | 0.000 | 0.003 | 0.009 | 0.035 | 0.022 | 0.070 | 0.098 | 0.190 | 0.464 |
| PCDHGA4_Asn587Lys | 0.000 | 0.077 | 0.000 | 0.004 | 0.007 | 0.032 | 0.028 | 0.062 | 0.080 | 0.178 | 0.457 |
| NHLRC1_Gly159Glu | 0.000 | 0.042 | 0.000 | 0.001 | 0.001 | 0.010 | 0.009 | 0.024 | 0.032 | 0.064 | 0.111 |
| RAB44_Pro1013Leu | 0.000 | 0.051 | 0.000 | 0.001 | 0.003 | 0.019 | 0.010 | 0.027 | 0.031 | 0.045 | 0.085 |
| EYS_Glu3160* | 0.000 | 0.051 | 0.000 | 0.001 | 0.006 | 0.019 | 0.016 | 0.028 | 0.047 | 0.080 | 0.205 |
| SENP6_Lys1003Ile | 0.000 | 0.040 | 0.000 | 0.002 | 0.005 | 0.013 | 0.019 | 0.032 | 0.044 | 0.101 | 0.204 |
| GRIK2_Arg873Cys | 0.000 | 0.038 | 0.000 | 0.001 | 0.007 | 0.018 | 0.016 | 0.037 | 0.046 | 0.119 | 0.215 |
| RNF217_Thr213Met | 0.000 | 0.052 | 0.000 | 0.002 | 0.004 | 0.012 | 0.010 | 0.030 | 0.040 | 0.112 | 0.210 |
| ADGB_His1632Tyr | 0.000 | 0.054 | 0.000 | 0.004 | 0.018 | 0.048 | 0.038 | 0.084 | 0.100 | 0.253 | 0.459 |
| SYNE1_Arg5956His | 0.000 | 0.057 | 0.000 | 0.002 | 0.005 | 0.017 | 0.017 | 0.033 | 0.044 | 0.094 | 0.199 |
| MTRF1L_Ile114Met | 0.000 | 0.036 | 0.000 | 0.000 | 0.005 | 0.046 | 0.009 | 0.042 | 0.044 | 0.153 | 0.334 |
| MAP3K4_Ile1554Asn | 0.000 | 0.044 | 0.000 | 0.003 | 0.010 | 0.026 | 0.021 | 0.052 | 0.063 | 0.157 | 0.329 |
| NPVF_Cys148Tyr | 0.000 | 0.104 | 0.000 | 0.003 | 0.013 | 0.044 | 0.031 | 0.079 | 0.092 | 0.238 | 0.553 |
| TAX1BP1_Thr210Ile | 0.000 | 0.088 | 0.000 | 0.002 | 0.011 | 0.048 | 0.039 | 0.097 | 0.107 | 0.246 | 0.566 |
| CDK14_Ala410Gly | 0.000 | 0.055 | 0.000 | 0.002 | 0.007 | 0.027 | 0.016 | 0.053 | 0.061 | 0.132 | 0.278 |
| XKR6_Arg343Gln | 0.000 | 0.053 | 0.000 | 0.003 | 0.006 | 0.028 | 0.015 | 0.039 | 0.057 | 0.174 | 0.364 |
| MMP16_Pro326Leu | 0.000 | 0.056 | 0.000 | 0.001 | 0.007 | 0.021 | 0.020 | 0.043 | 0.063 | 0.138 | 0.324 |
| TNFRSF11B_Arg242Trp | 0.000 | 0.000 | 0.000 | 0.000 | 0.000 | 0.000 | 0.000 | 0.000 | 0.000 | 0.000 | 0.000 |
| BNC2_Thr56Ile | 0.000 | 0.116 | 0.000 | 0.002 | 0.011 | 0.033 | 0.026 | 0.090 | 0.100 | 0.235 | 0.523 |
| CDKN2A_Trp110* | 0.000 | 0.114 | 0.000 | 0.002 | 0.009 | 0.035 | 0.024 | 0.058 | 0.097 | 0.202 | 0.461 |
| MAMDC2_Glu273Lys | 0.000 | 0.000 | 0.000 | 0.000 | 0.000 | 0.000 | 0.000 | 0.000 | 0.000 | 0.000 | 0.000 |
| SVEP1_His182Tyr | 0.000 | 0.047 | 0.000 | 0.002 | 0.006 | 0.033 | 0.016 | 0.039 | 0.050 | 0.151 | 0.323 |
| HUWE1_Arg1477Cys | 0.000 | 0.000 | 0.001 | 0.000 | 0.000 | 0.000 | 0.000 | 0.000 | 0.000 | 0.000 | 0.000 |
| ZXDA_Gly83Val | 0.000 | 0.046 | 0.000 | 0.004 | 0.008 | 0.021 | 0.010 | 0.034 | 0.041 | 0.073 | 0.193 |
| GRIA3_Val55Met | 0.000 | 0.047 | 0.000 | 0.000 | 0.004 | 0.024 | 0.015 | 0.044 | 0.040 | 0.074 | 0.181 |
| MAGEA8_Asp196Tyr | 0.000 | 0.159 | 0.000 | 0.004 | 0.019 | 0.042 | 0.030 | 0.088 | 0.123 | 0.289 | 0.618 |
| HMGB3_Arg10His | 0.000 | 0.131 | 0.000 | 0.003 | 0.011 | 0.045 | 0.042 | 0.081 | 0.128 | 0.274 | 0.582 |

Duplex read coverage over the course of treatment for patient G2 is shown in FIG. 3D and FIG. 3E. Mean read coverage for targets ranged from 3877×-4534× after consensus in cfDNA samples with >93% of targets (greater than 240 variants monitored) with >2000× duplex reads and >76% targets with >3000× duplex reads. The sequencing profile demonstrated high target coverage at high read depth.

Mutation allele frequency in cfDNA was monitored over the course of treatment for GRANITE patient G2. As shown in FIG. 3F, ctDNA was not detected above the lowest call threshold over the course of the treatment regimen for patient G2, correlating with a prolonged disease free period (no evidence of disease at any timepoint on study post-surgery). Accordingly, monitoring mutation allele frequency in cfDNA served as an effective non-invasive proxy for monitoring disease, including assessing the presence of a disease and disease burden.

TABLE 4
VAF Values from ctDNA of Patient G2

| | | D1D1 | D2D1 | D3D1A | D3D1B | D4D1 | D5D1 |
|---|---|---|---|---|---|---|---|
| Variants | gDNA | VAF | VAF | VAF | VAF | VAF | VAF |
| TRABD2B_A385T | 0.000 | 0.000 | 0.000 | 0.000 | 0.000 | 0.000 | 0.000 |
| ADAR_G751R | 0.000 | 0.000 | 0.000 | 0.000 | 0.000 | 0.000 | 0.000 |
| VILL_L273fs | 0.000 | 0.001 | 0.000 | 0.000 | 0.000 | 0.001 | 0.000 |
| SURF2_P146L | 0.000 | 0.000 | 0.000 | 0.000 | 0.000 | 0.000 | 0.000 |
| TP53_P153fs | 0.000 | 0.000 | 0.000 | 0.000 | 0.000 | 0.000 | 0.000 |
| CSH2_A156V | 0.000 | 0.000 | 0.000 | 0.000 | 0.000 | 0.000 | 0.000 |
| MAP2K2_E66K | 0.000 | 0.000 | 0.000 | 0.000 | 0.000 | 0.000 | 0.000 |

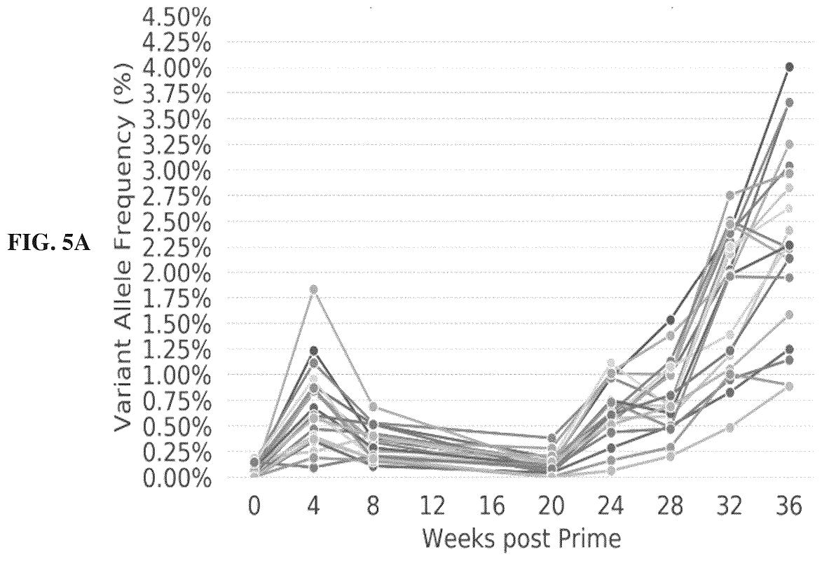

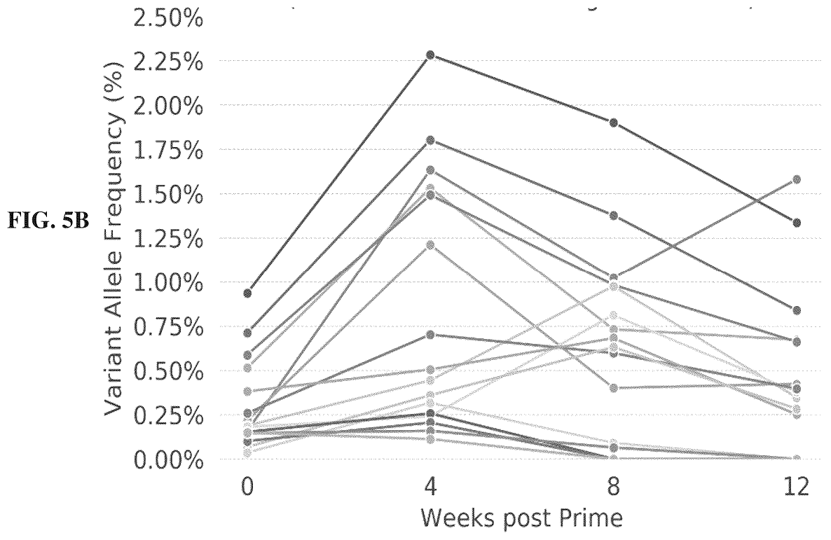

Mutation allele frequency in cfDNA was monitored over the course of treatment for GRANITE patients G3 and G8. FIG. 5A-B show the tracking of multiple variant alleles in each patient's ctDNA, respectively. Both patients showed a steady decrease in VAF following initial spikes around a month following initial treatment. In both patients, this decrease was associated with an overall reduction in tumor volume. Patient G3 demonstrated a maximum VAF reduction of 6-fold (average VAF 0.69% at week 4 vs average VAF 0.12% at week 20), with all 20 variants monitored detected. Patient G3's cfDNA profile correlated with disease progression at week 8, followed by stabilization by week 16, then minimally progressed at week 24 (T cell decline). Patient G8 demonstrated a continued decrease in mutant allele frequency, including loss of some variant detection (16 of 20 variants monitored detected), correlating with stable disease. Accordingly, monitoring mutation allele frequency in cfDNA served as an effective non-invasive proxy for monitoring status of disease, including assessing disease progression.

Mutation allele frequency in cfDNA was also monitored over the course of treatment for SLATE patient S1. The details of all ctDNA isolations are detailed in Table 5.

TABLE 5
Details of ctDNA Isolation from Patient
S1 receiving SLATE Therapy

| | Yield | Total | Plasma | |
|---|---|---|---|---|
| Sample | ng/uL | ng | mL | ng/mL |
| Dose 1 Day 1 | 1.43 | 143 | 8.00 | 17.88 |
| Dose 2 Day 1 | 0.988 | 98.8 | 8.00 | 12.35 |
| Dose 3 Day 1 | 1.06 | 106 | 8.00 | 13.25 |

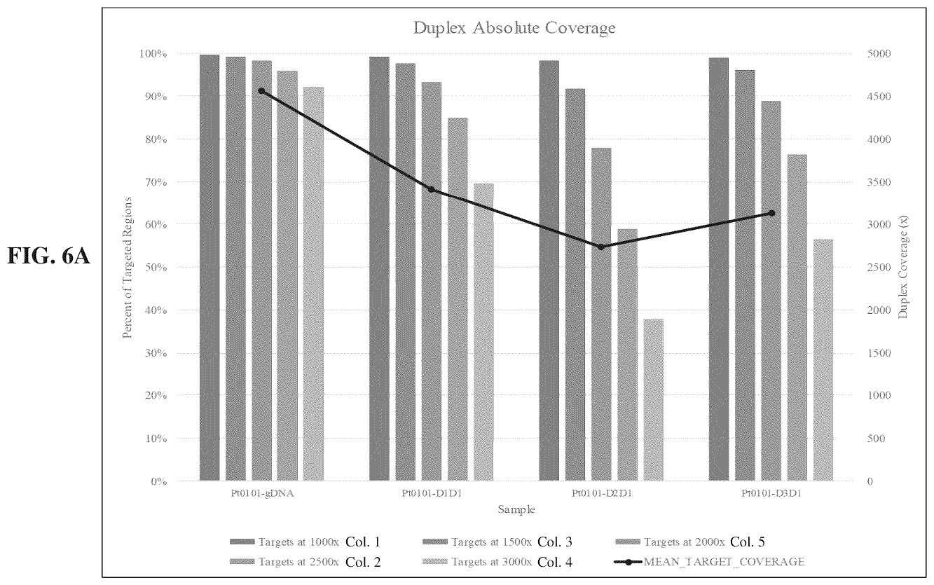

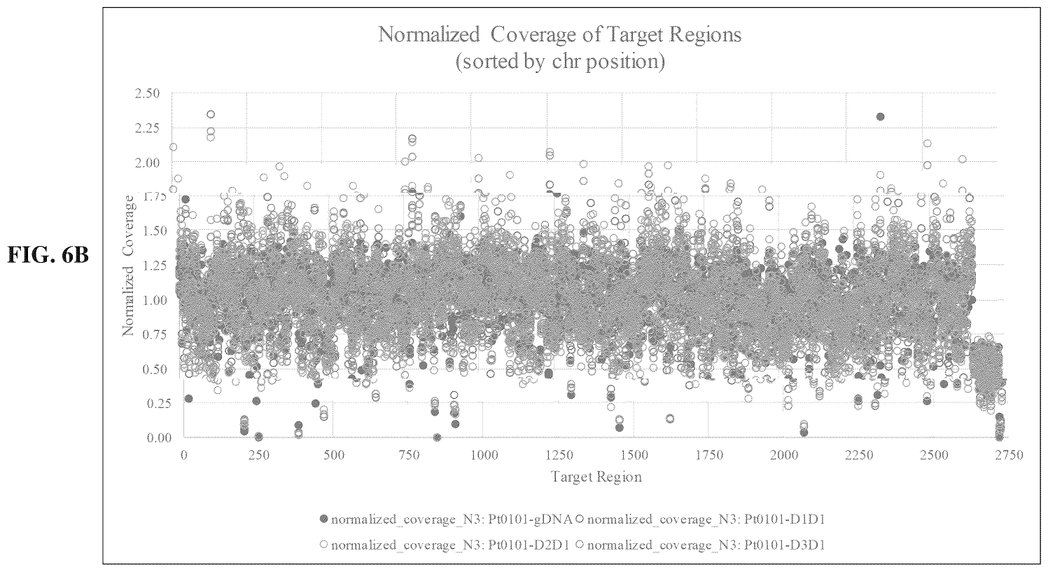

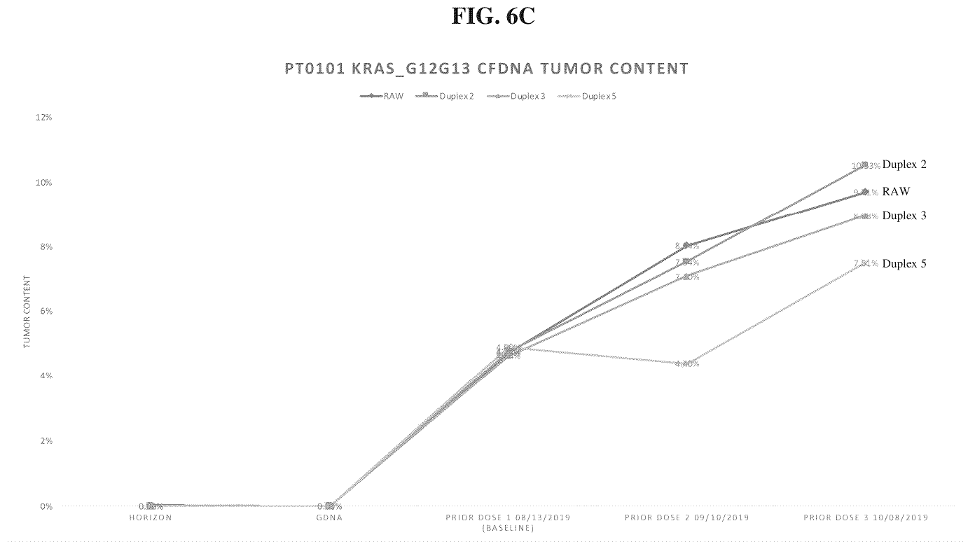

Duplex read coverage over the course of treatment for patient S1 is shown in FIG. 6A and FIG. 6B. Mean read coverage for targets ranged from 2728×-3660× after consensus in cfDNA samples with >98% of targets with >1000× duplex reads and >78% targets with >2000× duplex reads.

Mutation allele frequency in cfDNA was monitored over the course of treatment. As shown in FIG. 6C, a steady increase in ctDNA tumor content was observed is indicative of a progressing tumor. Results of all ctDNA analyses of patient S1 are given in Table 4. Accordingly, monitoring mutation allele frequency in cfDNA served as an effective non-invasive proxy for monitoring status of disease, including assessing disease progression.

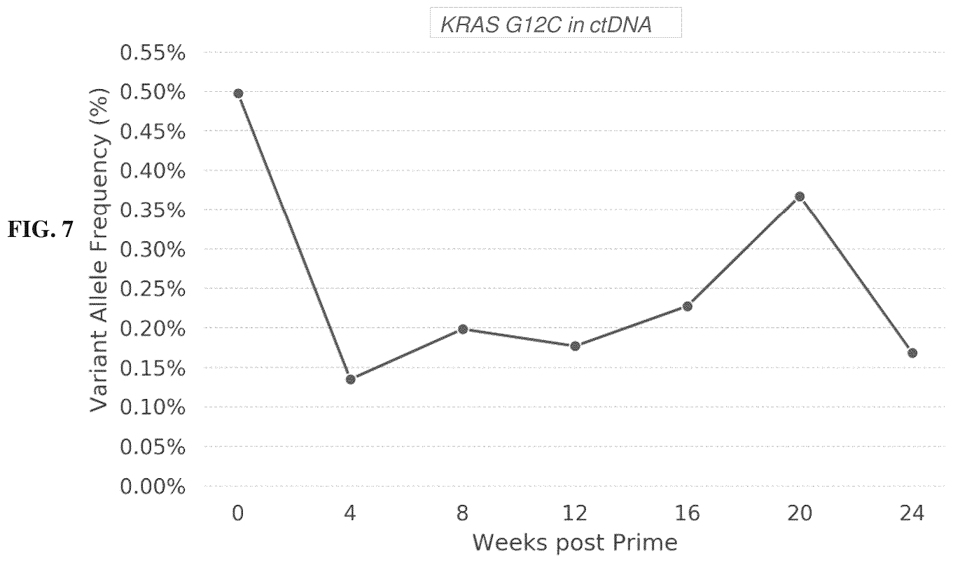

The tumor of SLATE patient S2 was determined to have a KRAS G12C mutation and variant-specific tracking of the KRAS G12C mutation was used for monitoring. As shown in FIG. 7, an overall decrease in VAF of the KRAS mutant was observed and correlated with a 20% reduction in tumor volume by week 8. Accordingly, monitoring mutation allele frequency in cfDNA served as an effective non-invasive proxy for monitoring status of disease, including assessing disease progression.

The results demonstrate mutation allele frequency in cfDNA could be monitored over the course of treatment for large numbers of tumor and subject-specific mutations. The results also demonstrated monitoring mutation allele frequency in cfDNA served as an effective non-invasive proxy for monitoring status of disease, including assessing disease progression, assessing the presence of a disease and disease burden, and the efficacy of a therapeutic regimen.

TABLE 6
Results of ctDNA Analyses from a Patient Receiving SLATE Therapy

| Sample | Duplex | Dose | WT | MUT | Tumor content | VAF |
|---|---|---|---|---|---|---|
| hz1pct-20 ng | 0 | NA | 66842 | 7 | 0.021% | 0.000 |
| (reference standard) | | | | | | |
| hz1pct-20 ng-Duplex-2 | 2 | NA | 2417 | 0 | 0.000% | 0.000 |
| (reference standard) | | | | | | |
| hz1pct-20 ng-Duplex-3 | 3 | NA | 2139 | 0 | 0.000% | 0.000 |
| (reference standard) | | | | | | |
| hz1pct-20 ng-Duplex-5 | 5 | NA | 1530 | 0 | 0.000% | 0.000 |
| (reference standard) | | | | | | |
| Pt0101-cfDNA-D1D1 | 0 | 1 | 56829 | 1377 | 4.731% | 0.024 |
| Pt0101-cfDNA-D1D1-Duplex-2 | 2 | 1 | 3067 | 75 | 4.774% | 0.024 |
| Pt0101-cfDNA-D1D1-Duplex-3 | 3 | 1 | 2441 | 58 | 4.642% | 0.023 |
| Pt0101-cfDNA-D1D1-Duplex-5 | 5 | 1 | 1393 | 35 | 4.902% | 0.025 |
| Pt0101-cfDNA-D2D1 | 0 | 2 | 44945 | 1883 | 8.042% | 0.040 |
| Pt0101-cfDNA-D2D1-Duplex-2 | 2 | 2 | 2781 | 109 | 7.543% | 0.038 |
| Pt0101-cfDNA-D2D1-Duplex-3 | 3 | 2 | 1875 | 69 | 7.099% | 0.035 |
| Pt0101-cfDNA-D2D1-Duplex-5 | 5 | 2 | 712 | 16 | 4.396% | 0.022 |
| Pt0101-cfDNA-D3D1 | 0 | 3 | 48414 | 2470 | 9.708% | 0.049 |
| Pt0101-cfDNA-D3D1-Duplex-2 | 2 | 3 | 2752 | 153 | 10.534% | 0.053 |
| Pt0101-cfDNA-D3D1-Duplex-3 | 3 | 3 | 1935 | 91 | 8.983% | 0.045 |
| Pt0101-cfDNA-D3D1-Duplex-5 | 5 | 3 | 872 | 34 | 7.506% | 0.038 |
| Pt0101-gDNA | 0 | NA | 97017 | 5 | 0.010% | 0.000 |
| Pt0101-gDNA-Duplex-2 | 2 | NA | 5190 | 0 | 0.000% | 0.000 |
| Pt0101-gDNA-Duplex-3 | 3 | NA | 4078 | 0 | 0.000% | 0.000 |
| Pt0101-gDNA-Duplex-5 | 5 | NA | 2262 | 0 | 0.000% | 0.000 |

### Example 2—Cell-Free DNA Monitoring with Combination Panels in GRANITE and SLATE Vaccine Programs

Circulating-tumor DNA (ctDNA) is an emerging, minimally-invasive diagnostic and prognostic biomarker for patients receiving immunotherapy. Example 2 describes the evaluation of ctDNA dynamics and tumor evolution over time through a combination approach of a tumor-informed and tumor-naïve ctDNA monitoring assay.

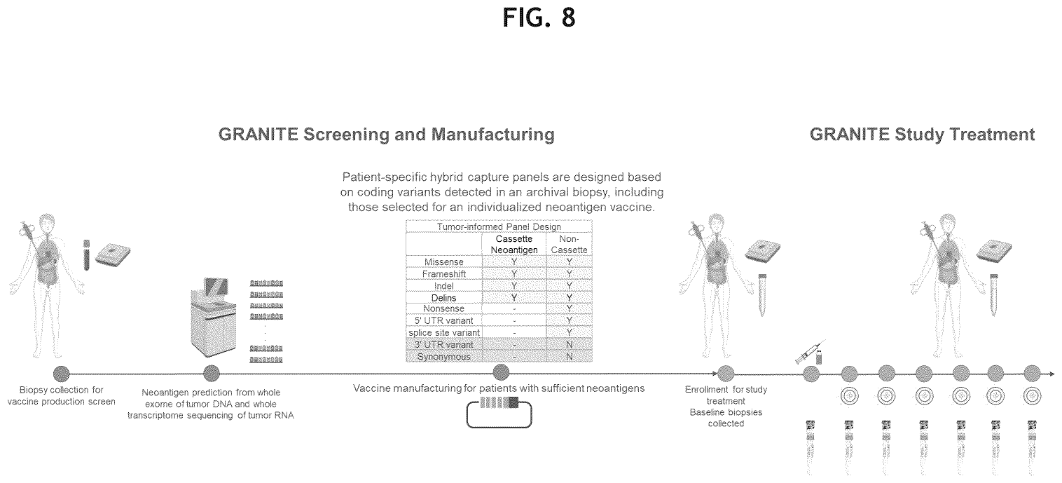

Methods describing the Cell-Free DNA Monitoring Assay are generally described in Example 1 and are briefly as follows: A patient biopsy was collected for the GRANITE vaccine production screen. Individualized vaccines were manufactured for patients with sufficient neoantigens. After patients were enrolled in the study baseline biopsies were collected. The patients were then administered the vaccines for treatment (“GRANITE” program). (FIG. 8). Certain subjects were instead administered an “off-the-shelf” shared neoantigen vaccine (“SLATE” program).

Combination probe panels for enriching targets were designed that included both tumor-informed and tumor-naïve probes. Neoantigens were predicted from whole exome sequencing (WES) of the tumor DNA and whole transcriptome sequencing of the tumor RNA. Tumor-informed patient-specific panels were designed for all coding mutations detected in whole exome sequencing (WES) of archival tissue (median: 123; range: 67-402), including patients selected for an individualized neoantigen vaccine program (GRANITE). A tumor-naïve panel (also referred to as a “universal” panel) was designed and included in patient panels to monitor recurrently mutated tumor hotspots and genes implicated in immunotherapy resistance. Specifically, the panel monitored genes and mutations generally considered oncogenic (e.g., “driver” mutations thought to promote cancer and generally considered gain-of-function mutations), tumor-suppressor genes (e.g., genes generally considered to monitor and/or control tumor-associated properties, such as cell division, where mutations can interfere with controlling such properties and are generally considered loss-of-function mutations), interferon-γ signaling pathway genes (including JAK/STAT signaling pathway genes), antigen-processing pathway genes (including monitoring for HLA loss of heterozygosity), and mutations otherwise generally associated with cancer but may not be otherwise annotated (e.g., not yet annotated as an oncogene or tumor-suppressor). Probes in the panel were designed to either monitor the full exon coding-regions of select genes, or designed to monitor particular regions or mutations (“hotspots”) within a gene.

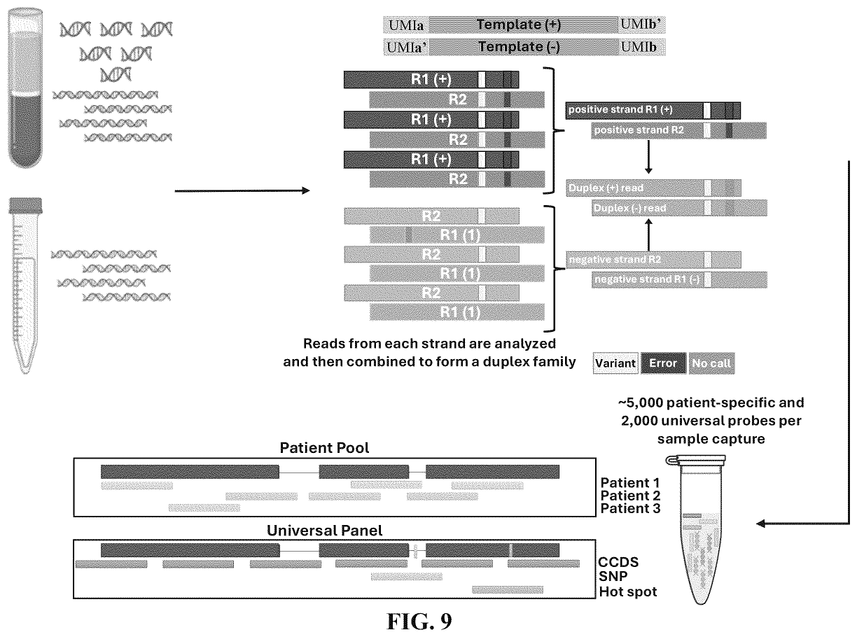

Monthly cell-free DNA (cfDNA) samples were collected on-treatment (mean 7; range: 1-18). Libraries with duplex unique molecular identifiers (UMIs) were prepared from cfDNA, matched normal DNA, and biopsy DNA and captured using a combination of personalized panels, a universal panel, or WES (FIG. 9). Shotgun libraries from cfDNA, biopsy DNA, or gDNA from whole blood or PBMCs were prepared with duplex UMIs. Duplex sequencing reduced noise by requiring variants to be observed on both strands of a duplex molecule. Enriched duplex libraries were sequenced to a mean target depth of >65,000× prior to consensus deduplication.

Patient-specific variants and universal regions were enriched for de novo variant calling. Patient specific regions were tumor-informed while the universal panel was tumor naïve. Multiple patient-specific sets were combined to create a superset containing probes for 6-9 patients. The universal panel captured a set of common targets in all patient samples (see, e.g., Table 7). The combination of panels is not only patient-specific but also provides flexibility to observe variants that were not present in the original tumor.

A summary of the GRANITE cfDNA monitoring assay is also provided in Table 6.

TABLE 6
GRANITE cfDNA Monitoring Assay

| Attribute | Description |
|---|---|
| Assay Type | Tumor-informed with tumor-naïve regions |
| Sample type | cfDNA |
| | Fragmented (enzymatic or mechanical), high- |
| | quality DNA from PBMCs, cells, or tissue |
| Sample input | ≥5 ng; |
| | 30 ng input target (9,000 haploid genomes) |
| UMI | Duplex |
| Enrichment | Hybrid capture |

Table 7 provides a summary of the cancer-associated genes and hotspots monitored in the universal, tumor-naïve panel. The panel also included about 290 probes configured to capture locations with common polymorphisms in the human population (SNPs) used for fingerprinting to uniquely identify a subject, e.g., if multiple subject sequences are multiplexed.

TABLE 7
Exemplary Genes in a Universal Panel
Universal Panel

| Coding Exons | | | | | | |
|---|---|---|---|---|---|---|
| Antigen | IFNγ/JAK- | Tumor | Hotspots in Select Genes | | | |
| Presenting | STAT | Suppressors | ALK | ERBB2 | HRAS | NRAS |
| B2M | IFNGR1 | TP53 | APC | ERBB3 | KDR | PDGFRA |
| HLA-A | IFNGR2 | PTEN | AR | FBXW7 | KIT | PDGFRB |
| HLA-B | JAK1 | ARID1A | ATRX | FGFR1 | KRAS | PIK3CA |
| HLA-C | JAK2 | | BRAF | FGFR3 | MAP2K1 | RET |
| TAP1 | | | CREBBP | FLT1 | NOTCH1 | ROS1 |
| TAP2 | | | CTNNB1 | FLT3 | NOTCH2 | SMO |
| NLRC5 | | | EFR | GNAS | NOTCH3 | TERT |
| | | | | | | promoter |

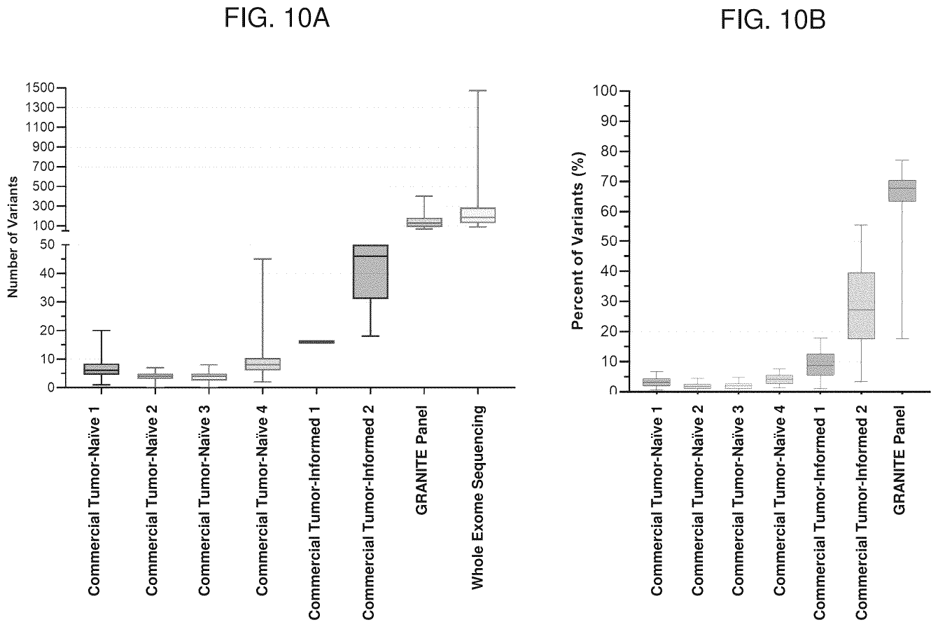

FIGS. 10A and 10B shows the variant coverage of manufacturing variants in the individualized GRANITE panel. FIG. 10A shows the number of potential variants covered by various commercially available NGS panels, including both separate tumor-naïve and tumor-informed, compared to the GRANITE panel designed and coverage by whole exome sequencing. FIG. 10B shows the percentage of WES variants potentially covered by the various NGS panels. The neoantigen coverage was determined by the variants present in the manufacturing biopsy of treated GRANITE patients. Without an archival biopsy, SLATE patients have only 1 mutation intentionally monitored (i.e., the tumor-informed mutation that was determined by sequencing that made the SLATE patient eligible to receive the SLATE vaccine). With the use of static panels, an average of 5-10 variants would be monitored in GRANITE patients. However, with the use of tumor-informed panels the range increases to 16-50 variants. Regardless of the panel type, the currently available assays would not overlap with the majority of tumor-specific neoantigens in the GRANITE vaccine.

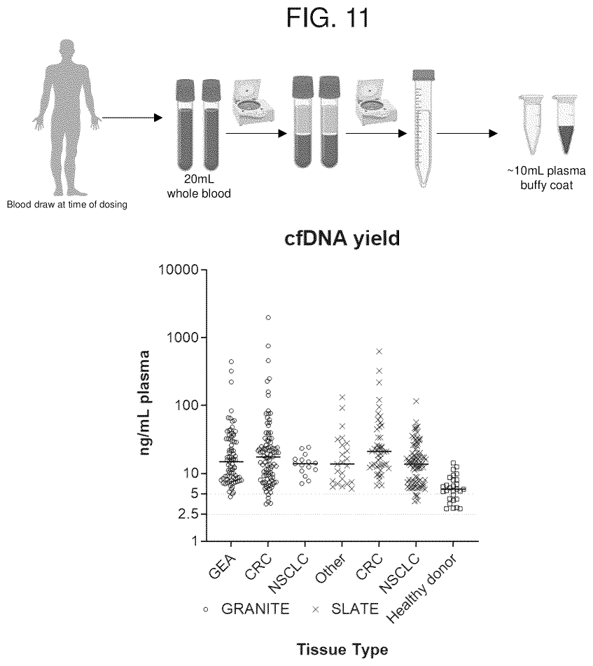

FIG. 11 shows the blood collection protocol for SLATE and GRANITE vaccine patients. Whole blood was collected at the time of vaccine dosing and centrifuged to collect the plasma serum and buffy coat layer. Most cfDNA results from the turnover of white blood cells. The collection protocol includes collection of the buffy coat and/or whole blood for sequencing of matched normal gDNA from the patient. The buffy coat collection allows for matched normal cfDNA to rule out clonal hematopoiesis via CHIP. Patients with advanced disease have a higher cfDNA concentration. Patient samples have a median yield of 15 ng/ml of plasma from whole blood. This was used to calculate hGE at 3000 he per 10 ng. The number of molecules limits the sensitivity. The cfDNA yield as ng/ml plasma collected from GEA, CRC, NSCLC, or other tumor tissue, or healthy donors in the GRANITE or SLATE patients is shown in FIG. 11.

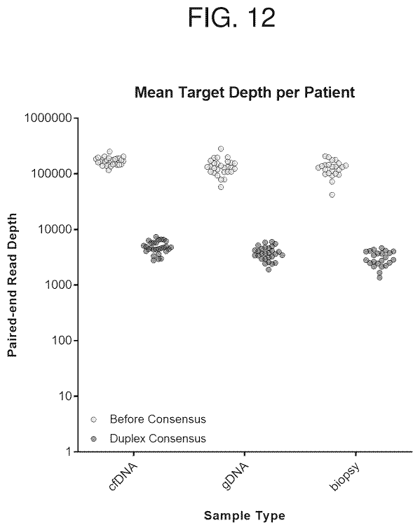

FIG. 12 shows that the GRANITE patient assay monitored an average of approximately 140 variants per patient at high sequencing depth for variant calling at >1000× duplex consensus coverage. Patient samples were sequenced to achieve a mean target depth of approximately 100,000× (paired end) depth, which was reduced to approximately 3900× depth after duplex consensus.

Table 8 provide a summary of the patient samples, including the tissue type, number of cfDNA samples, biopsies, number of variants targeted, and input cfDNA amount (ng) and the range of mean VAF of the longitudinal samples.

TABLE 8

| | | | | Number of | | |
|---|---|---|---|---|---|---|
| | | | | variants | | Range of mean |
| | | | Biopsies | targeted in | Input | VAF of |
| Patient | Tissue | cfDNA | for duplex | Gritstone | cfDNA | longitudinal |
| ID | type | samples | sequencing | assay | (ng) | samples |
| G01 | GEA | 18 | 2 | 237 | 12.4- | 0.25-49.6% |
| | | | | | 30.0 | |
| G02 | GEA | 12 | 0 | 241 | 20.0- | 0 |
| | | | | | 50.0 | |
| G03 | NSCLC | 8 | 2 | 110 | 28.3- | 0.12-2.81% |
| | | | | | 30.0 | |
| G04 | CRC | 2 | 1 | 91 | 30 | 18.0-35.4% |
| G05 | GEA | 1 | 1 | 189 | 30 | 22.4% |
| G06 | CRC | 6 | 1 | 80 | 30 | 2.35-16.5% |
| G07 | GEA | 3 | 0 | 76 | 28.8- | 0 |
| | | | | | 30.0 | |
| G08 | CRC | 17 | 1 | 180 | 30 | 0-10.8% |
| G09 | CRC | 9 | 1 | 118 | 30 | 4.93-25.3% |
| G10 | CRC | 5 | 1 | 135 | 30 | 12.3-25.4% |
| G11 | CRC | 17 | 1 | 67 | 11.6- | 1.18-5.23% |
| | | | | | 29.4 | |
| G12 | GEA | 6 | 2 | 91 | 30 | 1.17-11.7% |
| G13 | GEA | 11 | 1 | 76 | 24.4- | 0-0.27% |
| | | | | | 30.0 | |
| G14 | CRC | 14 | 2 | 85 | 28.6- | 0-8.88% |
| | | | | | 30.0 | |
| G15 | CRC | 3 | 1 | 259 | 30 | 33.8-40.6% |
| G16 | CRC | 14 | 0 | 95 | 22.1- | 0.23-1.20% |
| | | | | | 30.0 | |
| G17 | NSCLC | 3 | 1 | 402 | 25.4- | 0.20-0.22% |
| | | | | | 30.0 | |
| G18 | GEA | 3 | 1 | 131 | 30 | 23.6-44.3% |
| G19 | GEA | 1 | 1 | 96 | 30 | 3.59% |
| G20 | GEA | 2 | 1 | 181 | 24.8- | 2.14-21.2% |
| | | | | | 30.0 | |
| G21 | GEA | 8 | 2 | 212 | 17.6- | 0.30-2.02% |
| | | | | | 30.0 | |
| G22 | CRC | 6 | 0 | 92 | 25.0- | 0-1.25% |
| | | | | | 30.0 | |
| G23 | CRC | 1 | 1 | 128 | 30 | 22.7% |
| G24 | GEA | 9 | 2 | 139 | 30 | 1.39-19.8% |
| G25 | GEA | 4 | 1 | 134 | 26.2- | 0.58-2.74% |
| | | | | | 30.0 | |
| G26 | CRC | 0* | 0 | 76 | — | — |
| G27 | CRC | 3 | 1 | 84 | 30 | 5.22-15.5% |
| G28 | GEA | 4 | 1 | 134 | 25.0- | 1.42-8.23% |
| | | | | | 30.0 | |
| G29 | NSCLC | 5 | 1 | 251 | 30 | 0.16-3.19% |

| | | | | |
|---|---|---|---|---|
| Total | 196 | 30 | | |
| Mean | 7 | 1 | 144 | |
| Minimum | 0 | 0 | 67 | 12 |
| Maximum | 18 | 2 | 402 | 50 |

*Samples not collected

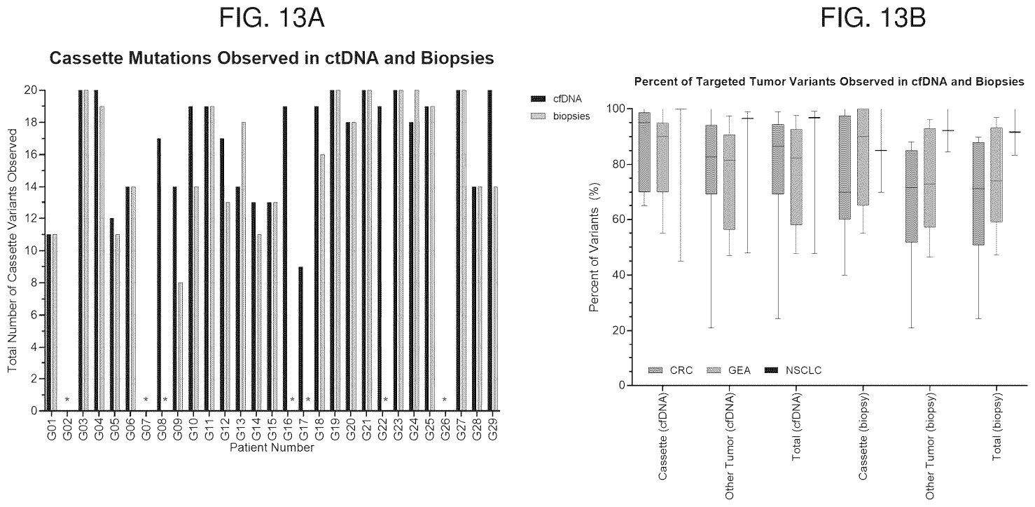

FIGS. 13A and 13B show that the majority of neoantigens were found in cfDNA and patient biopsies using the GRANITE assay. After multiple lines of treatment, the majority of neoantigens were found in the patients' cfDNA or tumor biopsies, indicating that many of the neoantigens were truncal variants appropriate for targeting with an individualized neoantigen vaccine. FIG. 13A shows the cassette mutations observed in the indicated patient's ctDNA and biopsies (* indicate patients with unavailable biopsies or where the tumor content was too low to detect variants in the assay). Significant overlap was found when comparing the variants in cfDNA and corresponding biopsies using the GRANITE assay, especially with the ability to call variants at a lower frequency in high-quality (RNALater or fresh frozen) biopsies (FIG. 13B).

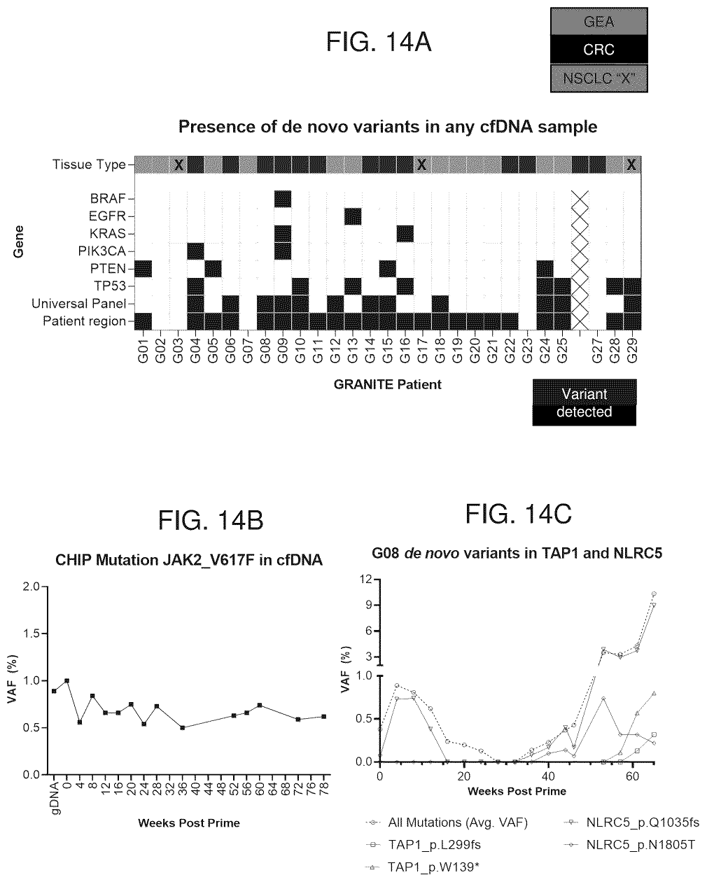

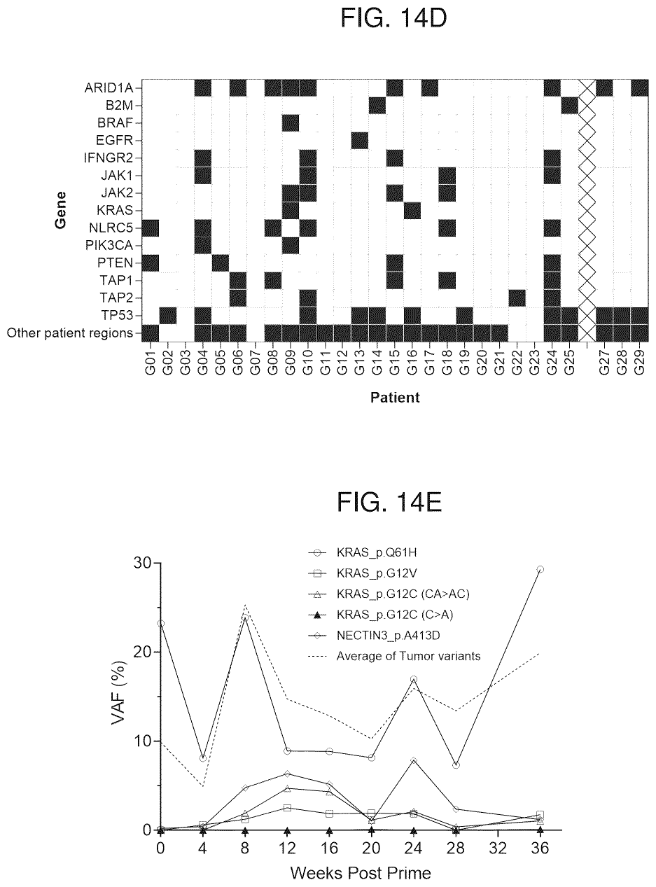

A universal panel based on tumor naïve regions captured the de novo variants after GRANITE vaccine treatment. FIG. 14A shows the presence of de novo variants in the cfDNA samples from the indicated patient and tumor tissue type (GEA, CRC, or NSCLC). Importantly, many patients had additional variants present in their cfDNA that were not in the original biopsy. The new variants often occurred where another patient had a targeted variant. Using the matched normal gDNA from whole blood or PMBCs, CHIP mutations were identified and ruled out as somatic tumor variants (FIG. 14B). For example, patient G08 had two NLRC5 mutations, one of which tracked with the average VAF of all variants, and two TAP1 mutations that appeared after nearly a year on therapy (FIG. 14C). FIG. 14D shows an additional analysis summary of variants observed in cfDNA that were found outside of the patient-specific variants demonstrating that 75% of the patients assessed had newly-detected variants in the cfDNA, including drivers (KRAS and BRAF) and resistance variants (TAP1). FIG. 14E shows that in patient G09, multiple, complex KRAS variants were detected that had not been detected in either the archival biopsy or the follow-up biopsy. The KRAS Q61H variant followed the same trajectory as the archival tumor variants whereas the three KRAS G12 variants occurred at a lower VAF with different dynamics, illustrating how multiple KRAS G12 hot spot variants can be captured with cfDNA to view metastatic disease. Accordingly, the tumor-naïve panel effectively monitored for additional mutations, including mutations potentially involved in immune evasion.

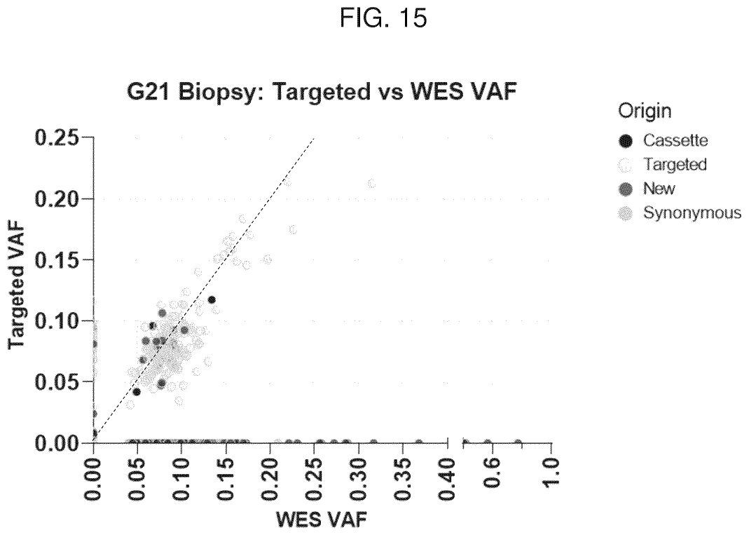

Duplex sequencing allowed for better sensitivity in biopsies. Targeted variants also provided insight into tumor heterogeneity. All patient-specific variants captured in WES of the biopsy were also captured using the patient-specific assay (100% concordance), see FIG. 15 and Table 9. A select number of de novo variants were observed in both assays, including new variants not captured by WES. Most de novo variants are not captured without performing unbiased WES. Despite their differences, the GRANITE monitoring assay captured 219/353 (62%) of the collection of variants observed between the two methods.

TABLE 9
Comparison of the patient specific variants captured
in the patient-specific cfDNA assay and WES

| | GRANITE | WES | Both | Variant Total |
|---|---|---|---|---|
| Cassette | 1 | 0 | 19 | 20 |
| Targeted | 37 | 0 | 148 | 185 |
| Coding | | | | |
| Synonymous | 0 | 45 | 0 | 45 |
| New | 7 | 89 | 7 | 103 |
| Total | 45 | 134 | 174 | 353 |

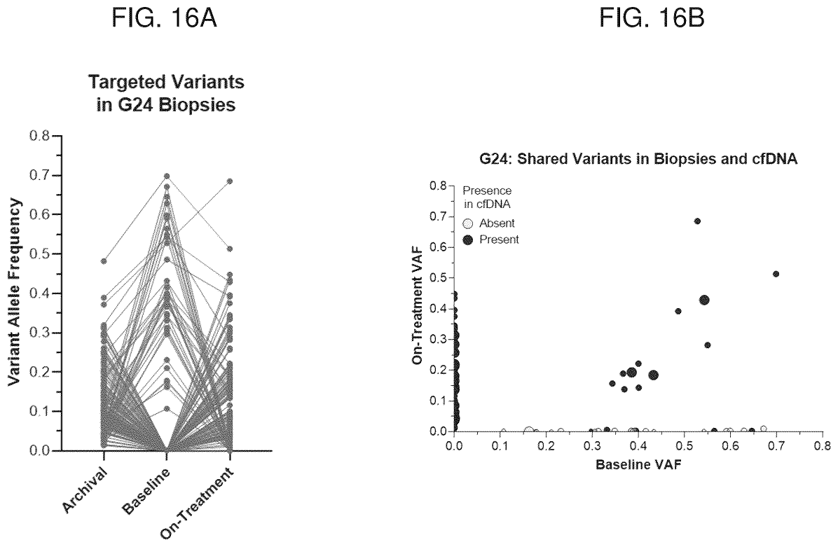

Using the monitoring assay on the patient's biopsies, the variants in the baseline biopsy were at a low frequency in the archival biopsy (FIG. 16A). The on-treatment biopsy variants were more representative of those present in the archival biopsy. Despite all biopsies being from the primary site, only 12/135 of the targeted variants were shared among the three, indicating tumor heterogeneity (FIG. 16B). In the cfDNA, 115 of the 135 variants were observed. Of the variants not observed, all but one were exclusive to the baseline biopsy (FIG. 16B).

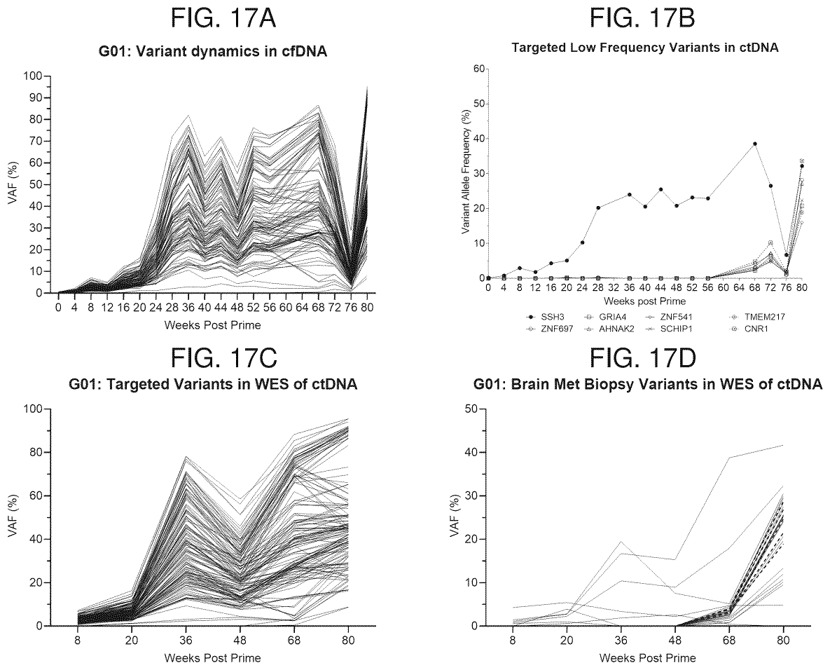

Exploratory analysis identified variants from brain metastasis in longitudinal cfDNA samples. A subset of variants were found to appear only near the end of treatment in the personalized assay, some of which were only found in the final biopsy (brain met). FIG. 17A shows the variant dynamics in cfDNA over time in patient G01. FIG. 17B shows the targeted low frequency variants in ctDNA for the indicated variants (SSH3, GRIA4, ZNF541, TMEM217, ZNF697, AHNAK2, SCHIP1, and CNR1). FIG. 17C shows the targeted variants in the WES of ctDNA of patient G01 over time. FIG. 17D shows the brain met biopsy variants in the WES of ctDNA of patient G01 over time. The brain met biopsy contained variants that were not found in biopsies taken earlier in treatment. Using WES of the cfDNA, the dynamics of the 32 variants could be followed. Dashed lines in FIG. 17D indicate variants that were also found in the patient-specific assay.

Thus, in 24 patients, a median of 92.5% of neoantigens (range: 45-100%) and a median of 84% (range: 24-99%) of all targeted variants were found in cfDNA. Indications of heterogeneity were found in both cfDNA and biopsies, and duplex sequencing improved target variant detection in low tumor content biopsies. Combining tumor-informed and tumor-naïve panels, de novo variants were found in the cfDNA of 19 patients. The de novo variants were discovered in regions targeted by the tumor-naïve panel or where another patient had a targeted variant. For instance, evidence of acquired immune escape was observed in a patient with colorectal cancer via biallelic TAP1 loss-of-function mutations. Using WES of longitudinal cfDNA in a patient with gastroesophageal adenocarcinoma, copy number changes, including HLA loss of heterozygosity, and emerging subclonal variants were corroborated between on-treatment biopsies and cfDNA.

Based on the results in Example 2, for patients treated with a neoantigen vaccine, longitudinal monitoring of cfDNA provided early insight into patients responding to treatment. Using a combination panel approach of tumor-informed and tumor-naïve monitoring, the ctDNA dynamics shown by targeting many mutations also tracked tumor burden, evolution, and emerging resistance. Specifically: (1) comprehensive tumor informed ctDNA monitoring provided improved breadth and maintained sensitivity to monitor longitudinal dynamics of tumor burden and vaccine delivered neoantigens in patients treated on a personalized cancer vaccine regimen (GRANITE); significant concordance in plasma and tumor biopsy collections was observed and demonstrated the ability of the ctDNA monitoring assay to detect and monitor variants in metastatic sites; and (3) combining a rationally designed tumor naïve panel detected and monitored important tumor intrinsic immune evasion events providing insight into the mechanism of action of individualized neoantigen immunotherapy.

### Example 3: Cell-Free DNA Monitoring with Combination Panels in SLATE Vaccine Treatment

### Circulating Tumour (Ct)DNA Sequencing and Analysis Methods

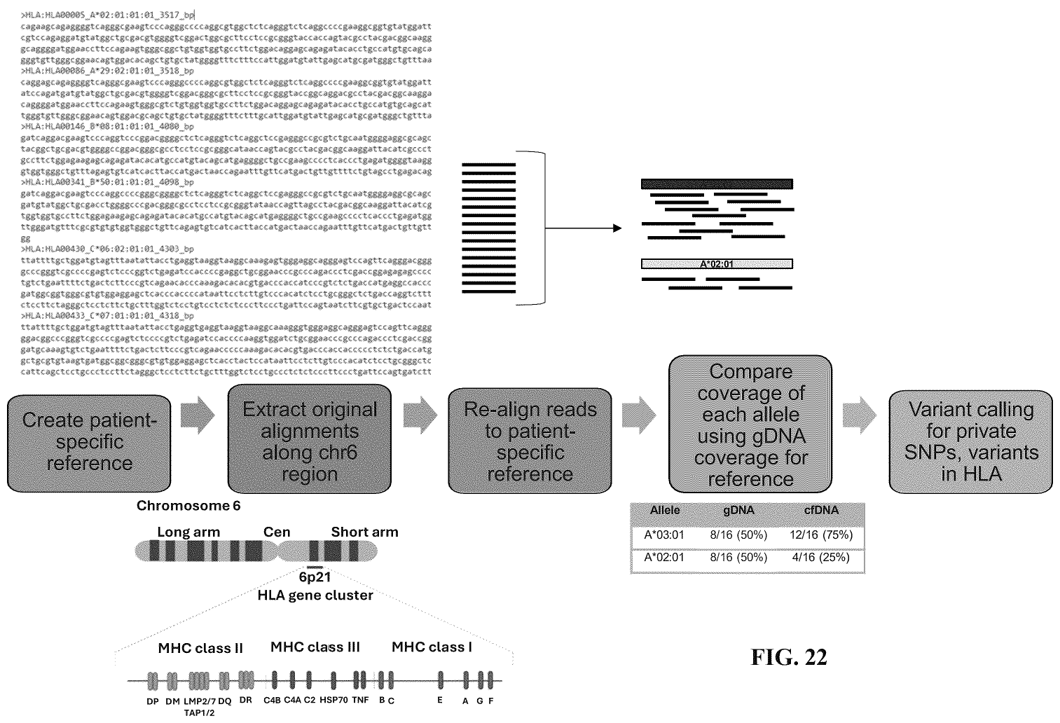

A universal set of capture probes were designed to capture mutations targeted by an “off-the-shelf” shared neoantigen vaccine cassette (“SLATE”), oncogenic hot spots, and SNPs for fingerprinting. Vaccine cassette design and manufacturing were performed as previously described (Palmer et al. “Individualized, heterologous chimpanzee adenovirus and self-amplifying mRNA neoantigen vaccine for advanced metastatic solid tumors: phase 1 trial interim results.” Nat Med. Aug. 15 2022; herein incorporated by reference for all purposes). Certain genes included probes designed to capture the entire coding region, such as for TP53, PTEN, ARID1A, and genes involved in the antigen presentation machinery (B2M, TAP1/2, and HLA-A, B, and C) were also designed for capture in the panel. FIG. 22 shows the general strategy for monitoring chromosome 6 for loss-of-heterozygosity for HLA genes.

The probes were designed and synthesized by Integrated DNA Technologies (IDT). Patient-matched genomic DNA from whole blood or PMBCs was fragmented prior to library preparation using the NEB FS module (NEB, Ipswich, MA). Shotgun libraries for cfDNA (up to 30 ng) and the fragmented, patient-matched genomic DNA (20-30 ng) were prepared using the KAPA HyperPrep (KAPA Biosystems, Wilmington, MA) kit using a customized pool of duplex adaptors containing unique molecular identifiers (UMI) for duplex sequencing (IDT). Shotgun libraries were captured overnight using the IDT xGen Hybridization and Wash kit. Enriched libraries were sequenced on an Illumina NovaSeq to a minimum mean raw depth of 65,000×. Briefly, UMIs were clipped from the raw sequencing reads prior to alignment to hg38 using BWA-MEM. Using fgbio, aligned reads were grouped by position and duplex identity. Consensus reads were created using a duplex of 3× (three supporting reads from each strand) and re-aligned to hg38. Variant calling was performed using FreeBayes and VarDictJava. Percent change in ctDNA was calculated as the change of the VAF of the SLATE variant for enrolment from the baseline sample.

Effective Control of Tumor Growth Observed in a Subset of Patients Treated with SLATEv1

The clinical activity of the SLATE vaccine regimen was assessed via the secondary endpoints of the Phase 1 study, ORR and PFS using RECIST v1.1 criteria, as well as OS. Eight out of 19 patients (42%) had a best overall response (BOR) of stable disease (SD), including 4 patients with NSCLC, 2 patients with PDA, 1 patient with CRC, and 1 patient with pancreatobiliary adenocarcinoma, with all other patients having a BOR of PD (Supplementary Table 2). The median PFS for all Phase 1 patients was 1.9 months (95% confidence interval (CI)=[1.7, 3.9 months]). The median OS across all tumor types was 7.9 months (95% CI=[4.7, 10.9 months]) (data not shown). 79% of patients (15/19) treated in Phase 1 demonstrated an OR of PD, with all progressing early in the treatment course, when the neoantigen-specific T cell response is still being generated (11/15 within 2 months and 4/15 within 4 months post the first vaccination). Despite many patients progressing quickly, 2/4 patients with NSCLC and SD, whose tumors had previously progressed on ICB, had decreases in target lesions indicative of tumor cell lysis by vaccine induced T cells.

While CT scans can provide insights into anti-tumor effects of cytotoxic treatments, effects with immunotherapies that induce robust T cell responses may be miscategorized due to T cell infiltration into the tumor and subsequent antigen-induced expansion, that may lead to increases in lesion size. The monitoring of ctDNA in blood, which has been shown to correlate with clinical outcomes, such as PFS and OS17-19 provides an alternative to CT scans for the longitudinal assessment of anti-tumor effects for patients treated with immunotherapies. In fact, recent evidence suggests that reduction in ctDNA may be a more sensitive marker of early treatment effects with immunotherapy and is better correlated with improved survival outcomes compared to imaging. Therefore, the level of ctDNA corresponding to the targeted neoantigen within the vaccine cassette was assessed as an exploratory endpoint using a tumor-informed probe, e.g., to monitor neoepitope encoded by the SLATE cassette that made the subject eligible for the trial. A molecular response (MR) characterized by a ≥30% reduction in neoantigen-specific ctDNA compared to baseline levels was observed in 23% of patients evaluable for MR (4/17), two of whom also demonstrated SD despite having progressed on prior ICB, suggesting effective immune control of tumor growth driven by vaccine elicited T cells in a subset of patients (data not shown).

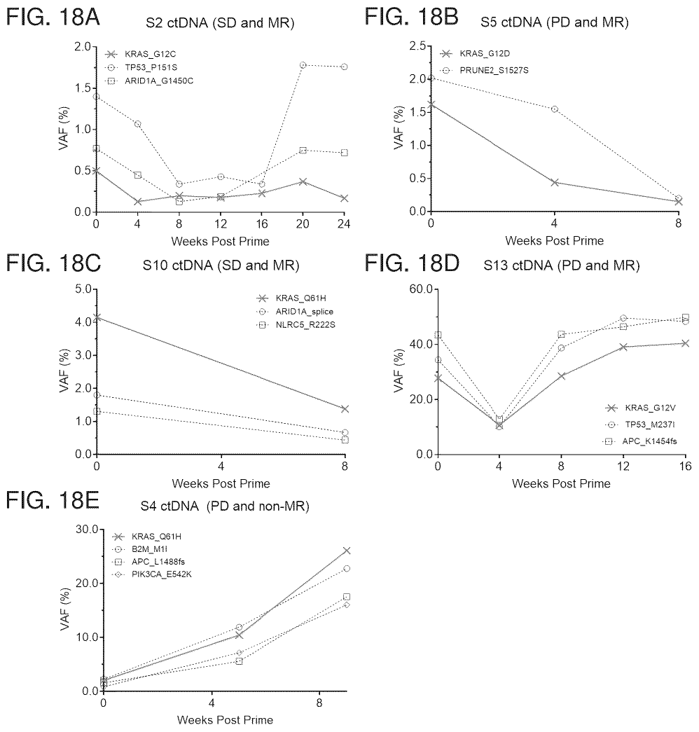

In addition to a reduction in the ctDNA level of the vaccine targeted neoantigen, a decrease in ctDNA for additional variants not encoded by the vaccine, e.g., monitored by additional tumor-informed probes for mutations of epitopes predicted to be presented by the subject's HLA and/or additional mutations commonly associated with cancer (e.g., driver mutations) was observed in all four patients with molecular response (MR) (FIG. 18A-E), providing further evidence for effective tumor targeting by vaccine induced T cells specific to one of the tumor neoantigens. FIG. 18A-E show ctDNA monitoring of tumor variants in SLATE patients. Levels of ctDNA over time post prime vaccination for each patient for the vaccine encoded tumor mutation (solid) as well as additionally detected somatic mutations (dashed) are shown. ctDNA levels reported as variant allele frequency (VAF) as a percentage of total reads. FIG. 18A shows the ctDNA % VAF for patient S2. FIG. 18B shows the ctDNA % VAF for patient S5. FIG. 18C shows the ctDNA % VAF for patient S10. FIG. 18D shows the ctDNA % VAF for patient S13. FIG. 18E provides a representative patient with no MR, showing loss of B2M start codon. SD=stable disease, PD=progressive disease, best overall response is denoted.

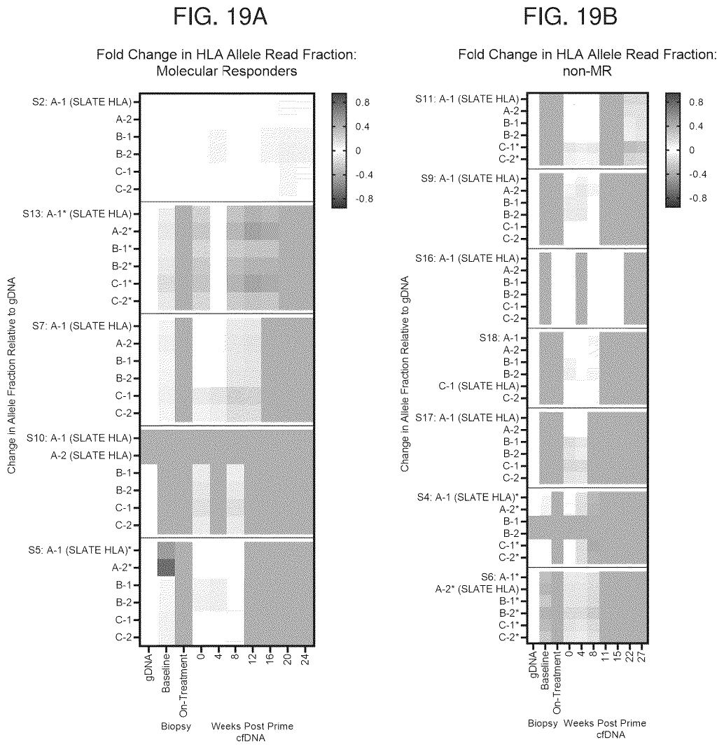

While the immune system can control tumor growth, tumors can escape immune control from a targeted immune response. One mechanism through which tumors have been shown to evade an immune response is through disruption of the antigen presentation pathway. A universal, tumor-naïve panel that included probes to monitor antigen presentation genes was included in the ctDNA panel to assess whether patients treated with SLATEv1 demonstrated defects in antigen presentation upon progression. Two patients, one a molecular responder and the other a molecular non-responder, demonstrated progressive loss-of-heterozygosity (LOH) of the relevant neoantigen matched HLA allele over the course of treatment (FIG. 19A-B) suggesting that the vaccine-induced T cells exerted immune pressure on the tumor, triggering this immune escape mechanism. FIG. 19A shows the fold change in the HLA allele read fraction from the molecular responder (MR). FIG. 19B shows the fold change in the HLA allele read fraction from the non-molecular responder (non-MR). One of the molecular responders, S13, showed some evidence of HLA LOH (p=0.015) in the baseline sample, which was not evident in the subsequent post-treatment sample corresponding with the drop in ctDNA. In all subsequent time points, evidence of HLA LOH was observed (p<0.01), which was associated with PD and suggests the outgrowth of tumor cells resistant to T cell control. The other patient, S4, had a variant resulting in the loss of the B2M start codon, which has been shown to reduce antigen presentation, detected at low frequency at baseline and increasing in frequency during treatment with the vaccine regimen (FIG. 18E). HLA LOH was also observed in HLA-A (the neoantigen-matched HLA allele) and HLA-C in the sample collected 9 weeks after initial vaccination (last collection). The B2M mutation and complete LOH may explain the lack of clinical activity observed in this patient. Together, these data demonstrate preliminary signs of clinical activity in a subset of patients with advanced/metastatic solid tumors treated with the SLATEv1 neoantigen vaccine.

### Example 4: Selection of Panel Probe Targets

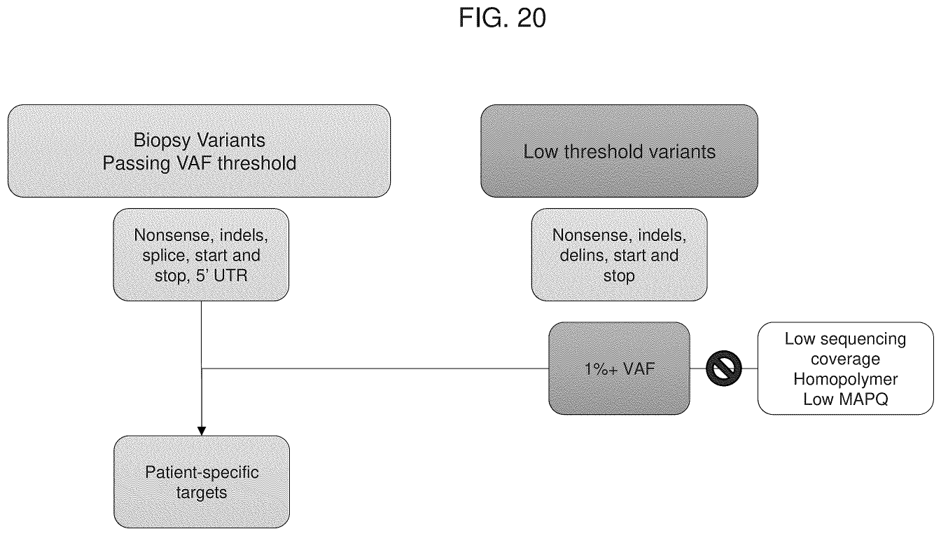

FIG. 20 provides a diagram outlining the considerations for inclusion of subject-specific, tumor-informed probes.

In addition, the universal panel targets in version 1 were further updated to target specific tissues types (e.g., CRC and NSCLC), resulting in universal panel version 2. The universal panel was updated by reviewing literature, TCGA, COSMIC, and MyCancerGenome datasets for hotspots and included monitoring genes and mutations generally considered oncogenic (e.g., “driver” mutations thought to promote cancer and generally considered gain-of-function mutations), tumor-suppressor genes (e.g., genes generally considered to monitor and/or control tumor-associated properties, such as cell division, where mutations can interfere with controlling such properties and are generally considered loss-of-function mutations), interferon-γ signaling pathway genes, JAK/STAT signaling pathway genes, antigen-processing pathway genes (including monitoring for HLA loss of heterozygosity), and mutations otherwise generally associated with cancer but may not be otherwise annotated (e.g., not yet annotated as an oncogene or tumor-suppressor). The initial results were prioritized based on prevalence, proximity, and oncogene/suppressor status, resulting in 133 priority regions. Examples of the Universal Panel version 1 targets are provided in Table 10.

TABLE 10
Universal Panel version 1
Probes Covering Full Coding Regions

| Antigen-processing | | IFNγ/JAK-STAT | | Tumor Suppressors |
|---|---|---|---|---|
| B2M | TAP1 | IFNGR1 | JAK1 | TP53 |
| HLA-A | TAP2 | INFGR2 | JAK2 | PTEN |
| HLA-B | NLRC5 | | | ARID1A |
| HLA-C | | | | |

| Hot spots | | | | SNPs (Fingerprinting) | |
|---|---|---|---|---|---|
| ABL1 | ERCC2 | MLH1 | ROS1 | AKT1 | GEN1 |
| AKT2 | ERCC5 | MMAB | SF3B1 | AXL | GNA11 |
| ALK | EXT1 | MSH3 | SF3B2 | BLM | IDH1 |
| APC | FANCA | MSH6 | SLX4 | BUB1B | IDH2 |
| AR | FANCD2 | MTOR | SMO | CCNE1 | JAK3 |
| ATR | FANCI | NF1 | TERT | CD79B | LDLR |
| | | | promoter | | |
| ATRX | FANCM | NFE2L2 | TET2 | CDH1 | MDM4 |
| BARD1 | FBXW7 | NOTCH1 | TP53BP1 | CDK4 | MLLT3 |
| BCL6 | FGF10 | NOTCH2 | TSC1 | CEP57 | MRE11 |
| BMPR1A | FGF6 | NOTCH3 | TSC2 | CSF1R | MSH2 |
| BRAF | FGFR1 | NRAS | WRN | DIS3L2 | NBN |
| BRCA1 | FGFR3 | NRG1 | XPA | EML4 | PIK3CD |
| BRCA2 | FLI1 | NTRK1 | XPC | EPCAM | PPP2R2A |
| BTK | FLT1 | NTRK3 | ZNF395 | ERCC1 | PRKAR1A |
| CARD11 | FLT3 | PDGFRA | | ERCC4 | PTPN11 |
| CCND1 | GNAS | PDGFRB | | ERG | RAD51B |
| CCND3 | HNF1A | PIK3CA | | ETS1 | RB1 |
| CDK12 | HRAS | PIK3CG | | ETV4 | RBM45 |
| CFH | KDR | PIK3R1 | | EWSR1 | RHBDF2 |
| CREBBP | KIT | PMS2 | | FANCE | RPS6KB1 |
| CTNNB1 | KRAS | PPARG | | FANCG | SDHB |
| DDR2 | MAGI1 | PROC | | FGF2 | SLTM |
| DNMT3A | MAP2K1 | PTCH1 | | FGF3 | SMARCB1 |
| EGFR | MAP2K2 | RAD54L | | FLCN | SMOX |
| EP300 | MAX | RAF1 | | FZR1 | STK11 |
| ERBB2 | MED12 | RECQL4 | | GATA2 | TFRC |
| ERBB3 | MET | RET | | GATA3 | |

~290 SNPs for fingerprinting; 115 kb footprint

The updated Universal Panel version 2 is provided in Table 11.

TABLE 11
Universal Panel version 2 (CRC/NSCLC-focused)
Probes Covering Full Coding Regions

| | | |
|---|---|---|
| Antigen-processing | IFNγ/JAK-STAT | Tumor Suppressors |

| | | | | | |
|---|---|---|---|---|---|
| B2M | HLA-E | IFNGR1 | JAK1 | TP53 | ARID1A |
| HLA-A | TAP1 | INFGR2 | JAK2 | PTEN | APC |
| HLA-B | TAP2 | | | | |
| HLA-C | NLRC5 | | | | |

| | | | | | Antigen- | SNPs | |
|---|---|---|---|---|---|---|---|
| Hot spots | | | | | processing | (Fingerprinting) | |
| ABCA12 | CFH | FGFR1 | NOTCH1 | RPL22 | CALR | AKT1 | GEN1 |
| ABL1 | COL12A1 | FGFR3 | NOTCH2 | RYR2 | CANX | AXL | GNA11 |
| ACVR2A | CREBBP | FHOD3 | NOTCH3 | SF3B1 | PSMB2 | BLM | IDH1 |
| AKAP9 | CSMD3 | FLG | NRAS | SLX4 | TAPBP | BUB1B | IDH2 |
| AKT2 | CTNNB1 | FLT1 | NRG1 | SMAD2 | | CCNE1 | JAK3 |
| ALK | DDR2 | FLT3 | NTRK1 | SMAD3 | | CD79B | LDLR |
| AMER1 | DNAH5 | GNAS | NTRK3 | SMAD4 | | CDH1 | MDM4 |
| AR | DNMT3A | HNF1A | OBSCN | SMO | | CDK4 | MLLT3 |
| ASXL1 | DOCK3 | HRAS | PCBP1 | SOX9 | | CEP57 | MRE11 |
| ATM | EGFR | KDR | PCLO | STAT1 | | CSF1R | MSH2 |
| ATR | EP300 | KIT | PDGFRA | SYNE1 | | DIS3L2 | NBN |
| ATRX | ERBB2 | KRAS | PDGFRB | TAPBP | | EML4 | PIK3CD |
| AXIN2 | ERBB3 | MAGI1 | PIK3CA | TCERG1 | | EPCAM | PPP2R2A |
| BARD1 | ERCC2 | MAP2K1 | PIK3CG | TCF7L2 | | ERCC1 | PRKAR1A |
| BCL6 | ERCC5 | MAP2K2 | PIK3R1 | TERT | | ERCC4 | PTPN11 |
| | | | | Promoter | | | |
| BCL9L | EXT1 | MAX | PLEKHA6 | TET2 | | ERG | RAD51B |
| BMPR2 | FANCA | MDN1 | PROC | TGFBR2 | | ETS1 | RB1 |
| BRAF | FANCD2 | MECOM | PSMB2 | TP53BP1 | | ETV4 | RBM45 |
| BRCA1 | FANCI | MED12 | PTCH1 | TSC1 | | EWSR1 | RHBDF2 |
| BRCA2 | FANCM | MET | PTPRT | TSC2 | | FANCE | RPS6KB1 |
| BTK | FAT1 | MLH1 | RAD54L | WNT16 | | FANCG | SDHB |
| CALR | FAT2 | MMAB | RECQL4 | WRN | | FGF2 | SLTM |
| CANX | FAT3 | MSH3 | RELN | XPC | | FGF3 | SMARCB1 |
| CARD11 | FAT4 | MSH6 | RET | ZBTB20 | | FLCN | SMOX |
| CASP8 | FBXW7 | MTOR | RNF43 | ZDBF2 | | FZR1 | STK11 |
| CCND1 | FGF10 | NBEA | ROBO1 | ZFP36L2 | | GATA2 | TFRC |
| CCND3 | FGF6 | NF1 | ROS1 | ZNRF3 | | GATA3 | |

~290 SNPs for fingerprinting; 135 kb footprint

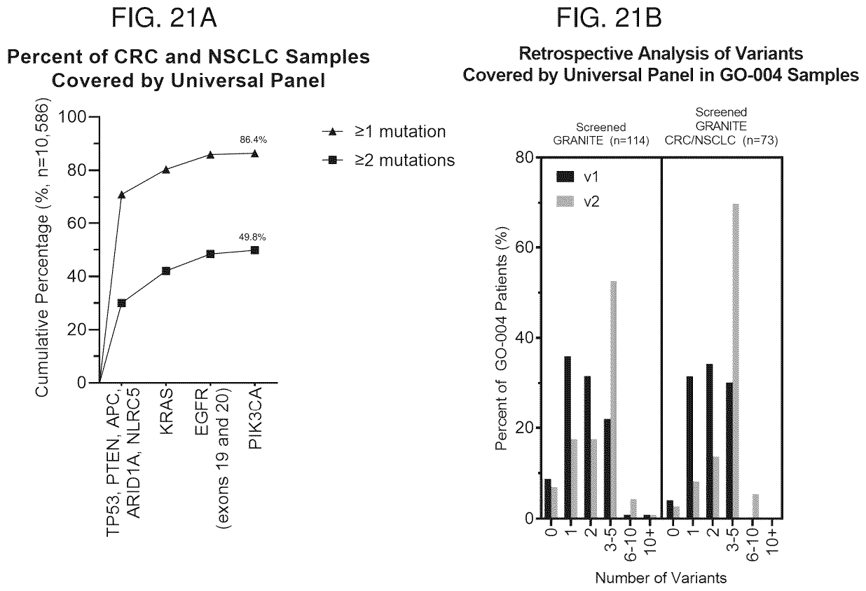

FIG. 21A provides the percentage of CRC and NSCLC samples that are covered by the target probes in the Universal Panel. Up to 86.4% of all samples with more than or equal to one mutation are covered by the universal panel. Up to 49.8% of all samples with more than or equal to two mutations are covered by the universal panel. The data is based on analysis of 10,586 samples from cbioportal.org. FIG. 21B shows a retrospective analysis of the variants in prior study (GO-004) patients identified by the universal panels version 1 (v1) or version 2 (v2). A summary of the data is provided in Table 12.

TABLE 12
Variants covered by Universal Panel v1 and v2.

| Variants Covered | |
|---|---|
| Screened GRANITE | Screened GRANITE |
| (n = 114) | CRC/NSCLC (n = 73) |

| | | | | |
|---|---|---|---|---|
| Mean | 3.4 | 5.2 | 2 | 3.3 |
| Median | 2 | 3 | 2 | 3 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A panel of polynucleotide probes for enriching cfDNA, the panel comprising:
(A) one or more tumor-informed polynucleotide probes; and
(B) one or more tumor-naïve polynucleotide probes.

2. The panel of claim 1, wherein the one or more tumor-informed polynucleotide probes are configured to capture a target sequence comprising an epitope sequence encoded by a cancer vaccine administered to a subject, wherein the subject has been determined to have a tumor expressing the epitope sequence.

3. The panel of claim 2, wherein the epitope sequence comprises;
a) a KRAS mutation, optionally wherein the KRAS mutation is selected from the group consisting of a KRAS_G12C mutation, a KRAS_G12D mutation, a KRAS_G12V mutation, and a KRAS_Q61H mutation;
b) a mutation selected from the group consisting of: KRAS_G13D, KRAS_Q61K, TP53_R249M, CTNNB1_S45P, CTNNB1_S45F, ERBB2_Y772 A775dup, KRAS_G12D, KRAS_Q61R, CTNNB1_T41A, TP53_K132N, KRAS_G12A, KRAS_Q61L, TP53_R213L, BRAF_G466V, KRAS_G12V, KRAS_Q61H, CTNNB1_S37F, TP53_S127Y, TP53_K132E, and KRAS_G12C:
c) an EGFR mutation, optionally wherein the EGFR mutation comprises an EGFR_L858R mutation; or
d) one or more subject-specific epitopes, wherein the tumor of the subject has been sequenced to determine the subject-specific epitopes to be encoded by the cancer vaccine, optionally wherein:
i) the one or more subject-specific epitopes comprises at least 2 subject-specific epitopes, at least 10 subject-specific epitopes, at least 20 subject-specific epitopes, or between 2-20 subject-specific epitopes, or
ii) the one or more subject-specific epitopes comprises between 2-20 subject-specific epitopes.

4-10. (canceled)

11. The panel of claim 2, wherein the panel further comprises additional tumor-informed polynucleotide probes that capture additional target sequences, wherein the tumor has been determined to express the additional target sequences, and wherein the additional target sequences are not encoded by the cancer vaccine, optionally wherein:
a) the additional target sequences comprise at least 10 target sequences, at least 20 target sequences, at least 30 target sequences, at least 100 target sequences, between 10-500 target sequences, between 30-500 target sequences, between 100-500 target sequences, between 10-100 target sequences, between 30-100 target sequences, or between 100-100 target sequences; and/or
b) the additional target sequences have been predicted to be presented by at least one HLA of the subject.

12-13. (canceled)

14. The panel of claim 1, wherein the one or more tumor-naïve polynucleotide probes are configured to capture a target sequence comprising a sequence of interest selected from:
(A) the group consisting of: a cancer-associated gene, an oncogene, a tumor-suppressor gene, an interferon-T signaling pathway gene, an antigen-processing pathway gene, and combinations thereof, optionally wherein:
(i) the cancer-associated gene:
(a) is selected from the group consisting of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2, or
(b) comprises each of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, and ZDBF2;
(ii) the oncogene:
(a) is selected from the group consisting of: ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20, or
(b) comprises each of: ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, and ZBTB20;
(iii) the tumor-suppressor gene:
(a) is selected from the group consisting of: TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3, or
(b) comprises each of: TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, and ZNRF3;
(iv) the interferon-γ signaling pathway gene:
(a) is selected from the group consisting of: IFNGR1, INFGR2, JAK1, JAK2, and STAT1, or
(b) comprises each of: IFNGR1, INFGR2, JAK1, JAK2, and STAT1; and/or
(v) the antigen-processing pathway gene:
(a) is selected from the group consisting of: B2M, HLA-A, HLA-B, HLA-C, HLA-E, TAP1, TAP2, NLRC5, CALR, CANX, PSMB2, and TAPBP, or
(b) comprises each of: B2M, HLA-A, HLA-B, HLA-C, HLA-E, TAP1, TAP2, NLRC5, CALR, CANX, PSMB2, and TAPBP;
optionally each of a cancer-associated gene, an oncogene, a tumor-suppressor gene, an interferon-γ signaling pathway gene, and an antigen-processing pathway gene;
optionally wherein:
(B) the group consisting of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2, TAPBP, and combinations thereof;
optionally each of: ABCA12, ACVR2A, AKAP9, BMPR2, COL12A1, CSMD3, DNAH5, DOCK3, FAT2, FAT3, FAT4, FGF10, FGF6, FLG, MAGI1, MDN1, MMAB, NBEA, OBSCN, PCBP1, PCLO, PLEKHA6, PROC, RAD54L, RELN, RPL22, RYR2, TCERG1, WRN, ZDBF2, ABL1, AKT2, ALK, AR, BCL6, BCL9L, BRAF, BTK, CARD11, CCND1, CCND3, CTNNB1, DDR2, EGFR, ERBB2, ERBB3, FGFR1, FGFR3, FHOD3, FLT1, FLT3, GNAS, HRAS, KDR, KIT, KRAS, MAP2K1, MAP2K2, MECOM, MED12, MET, MTOR, NOTCH1, NOTCH2, NOTCH3, NRAS, NRG1, NTRK1, NTRK3, PDGFRA, PDGFRB, PIK3CA, PIK3CG, PSMB2, RET, ROS1, SF3B1, SMO, SYNE1, ZBTB20, TP53, PTEN, ARID1A, APC, AMER1, ASXL1, ATM, ATR, ATRX, AXIN2, BARD1, BRCA1, BRCA2, CASP8, CFH, CREBBP, DNMT3A, EP300, ERCC2, ERCC5, EXT1, FANCA, FANCD2, FANCI, FANCM, FAT1, FBXW7, HNF1A, MAX, MLH1, MSH3, MSH6, NF1, PIK3R1, PTCH1, PTPRT, RECQL4, RNF43, ROBO1, SLX4, SMAD2, SMAD3, SMAD4, SOX9, TCF7L2, TERT Promoter, TET2, TGFBR2, TP53BP1, TSC1, TSC2, WNT16, XPC, ZFP36L2, ZNRF3, IFNGR1, INFGR2, JAK1, JAK2, STAT1, B2M, HLA-A, HLA-B, HLA-C, HLA-E, TAP1, TAP2, NLRC5, CALR, CANX, PSMB2, and TAPBP; or
(C) each of: ABL1, AKT2, ALK, APC, AR, ATR, ATRX, BARD1, BCL6, BMPR1A, BRAF, BRCA1, BRCA2, BTK, CARD11, CCND1, CCND3, CDK12, CFH, CREBBP, CTNNB1, DDR2, DNMT3A, EGFR, EP300, ERBB2, ERBB3, ERCC2, ERCC5, EXT1, FANCA, FANCD2, FANCI, FANCM, FBXW7, FGF10, FGF6, FGFR1, FGFR3, FLI1, FLT1, FLT3, GNAS, HNF1A, HRAS, KDR, KIT, KRAS, MAGI1, MAP2K1, MAP2K2, MAX, MED12, MET, MLH1, MMAB, MSH3, MSH6, MTOR, NF1, NFE2L2, NOTCH1, NOTCH2, NOTCH3, NRAS, NRG1, NTRK1, NTRK3, PDGFRA, PDGFRB, PIK3CA, PIK3CG, PIK3R1, PMS2, PPARG, PROC, PTCH1, RAD54L, RAF1, RECQL4, RET, ROS1, SF3B1, SF3B2, SLX4, SMO, TERT promoter, TET2, TP53BP1, TSC1, TSC2, WRN, XPA, XPC, ZNF395, B2M, HLA-A, HLA-B, HLA-C, TAP1, TAP2, NLRC5, IFNGR1, INFGR2, JAK1, JAK2, TP53, PTEN, and ARID1A.

15-28. (canceled)

29. The panel of claim 1, wherein:
(A) the one or more tumor-naïve polynucleotide probes comprises two or more probes configured to capture all coding exon sequences of a given gene; and/or
(B) the one or more tumor-naïve polynucleotide probes comprises two or more probes configured to capture a genomic region of interest associated-with cancer; and/or
(C) the tumor-informed polynucleotide probes and/or the tumor-naïve polynucleotide probes comprise probes that comprise overlapping sequences; and/or
(D) the panel comprises at least 20 probes, at least 30 probes, at least 40 probes, at least 50 probes, at least 60 probes, at least 70 probes, at least 80 probes, at least 90 probes, at least 100 probes, at least 200 probes, at least 300 probes, at least 400 probes, or at least 500 probes; and/or
(E) the panel is configured to cover at least 100 kb, at least 300 kb, at least 300 kb, at least 400 kb, between 100-400 kb, between 200-400 kb, between 300-400 kb, between 100-500 kb, between 200-500 kb, between 300-500 kb, or between 340-400 kb of the subject's genome; and/or
(F) the one or more tumor-naïve polynucleotide probes comprises polynucleotide probes configured to capture sequences associated with a given cancer the subject is known to have or suspected of having, optionally wherein the cancer is CRC or NSCLC; and/or
(G) the panel further comprises additional polynucleotide probes configured to capture sequences comprising polymorphisms in the human population, wherein the sequences comprising polymorphisms are capable in combination of uniquely identifying the subject.

30-35. (canceled)

36. A method for enriching cfDNA, the method comprising:
(a) providing a sample comprising cfDNA;
(b) providing a panel of polynucleotide probes comprising the panel of claim 1;
(c) contacting the sample comprising cfDNA with the panel of polynucleotide probes under conditions sufficient for cfDNA comprising a target sequence of interest to hybridize with its respective polynucleotide probe; and
(d) capturing the hybridized cfDNA and polynucleotide probe pairs to enrich the cfDNA.

37. A method for monitoring cancer status in a subject having, had, or suspected of having cancer, wherein the method comprises;
(A) the steps of:
(a) obtaining or having obtained sequencing data of cell-free DNA (cfDNA) from a sample from the subject, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read depth is mean duplex read depth, and optionally wherein obtaining the sequencing data comprises collecting or having collected the sample from the subject, isolating or having isolated the cfDNA, enriching or having enriched the cfDNA, and/or sequencing or having sequenced the cfDNA; and
(b) determining or having determined a frequency of the mutations present in the exome to assess the status of the cancer, optionally wherein assessment of the status comprises assessment of presence and/or cancer burden,
wherein the cfDNA has been enriched prior to sequencing using (1) a panel of subject-specific polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest, or
(B) the steps of:
(a) obtaining or having obtained sequencing data of cell-free DNA (cfDNA) from a sample from the subject, and wherein the sequencing data comprises a target coverage of at least 95% of all polynucleotide regions of interest corresponding to mutations present in an exome of the cancer, wherein the polynucleotide regions of interest comprise at least 50 mutations, and wherein the sequenced polynucleotide regions of interest comprise duplex read depth of at least 1000×, and optionally wherein obtaining the sequencing data comprises collecting or having collected the sample from the subject, isolating or having isolated the cfDNA, enriching or having enriched the cfDNA, and/or sequencing or having sequenced the cfDNA; and
(b) determining or having determined a frequency of the at least 50 mutations present in the exome to assess the status of the cancer, optionally wherein assessment of the status comprises assessment of presence and/or cancer burden,
wherein the cfDNA has been enriched prior to sequencing using (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest,
optionally wherein the method comprises designing and/or selecting or having designed and/or selected a combination panel of a panel of tumor-informed polynucleotide probes and a panel of tumor-naïve polynucleotide probes, further optionally wherein the designed and/or selected combination panel comprises the panel of claim 1.

38. (canceled)

39. A method for assessing efficacy of a therapy in a subject, had, or suspected of having cancer, wherein the method comprises:
(A) the steps of:
(a) obtaining or having obtained sequencing data of cell-free DNA (cfDNA) from a pre-therapy sample from the subject, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read coverage is mean duplex read coverage, and optionally wherein obtaining the sequencing data comprises collecting or having collected the pre-therapy sample from the subject, isolating or having isolated the pre-therapy cfDNA, enriching or having enriched the pre-therapy cfDNA, and/or sequencing or having sequenced the pre-therapy cfDNA;
(b) obtaining or having obtained sequencing data of cell-free DNA (cfDNA) from a post-therapy sample from the subject, optionally wherein the therapy comprises a cancer vaccine comprising the neoantigen or expression system encoding the same, and wherein the sequencing data comprises a target coverage of at least 50% of all polynucleotide regions of interest corresponding to mutations present in an exome of the cancer and wherein the sequenced polynucleotide regions of interest comprise read depth of at least 1000×, optionally wherein the polynucleotide regions of interest comprise at least 50 mutations, optionally wherein the mean read coverage is mean duplex read coverage, and optionally wherein obtaining the sequencing data comprises collecting or having collected the post-therapy sample from the subject, isolating or having isolated the post-therapy cfDNA, enriching or having enriched the post-therapy cfDNA, and/or sequencing or having sequenced the post-therapy cfDNA; and
(c) determining or having determined the frequency the mutations present in the exome of the pre-therapy cfDNA relative to the post-therapy cfDNA to assess the efficacy of the therapy, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable,
wherein the cfDNA has been enriched prior to sequencing using (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest, or
(B) the steps of:
(a) obtaining or having obtained sequencing data of tumor-derived DNA from a cancer-diseased tissue from the subject, optionally wherein obtaining the sequencing data comprises collecting or having collected the cancer-diseased tissue, isolating or having isolated the tumor-derived DNA, and sequencing or having sequenced the tumor-derived DNA;
(b) determining or having determined one or more tumor-associated mutations relative to a wild-type germline nucleic acid sequence of the subject from the tumor-derived DNA sequencing data, optionally wherein one or more of the one or more tumor-associated mutations is associated with a neoantigen comprising at least one alteration that makes a peptide sequence encoded by the tumor-derived DNA distinct from the corresponding peptide sequence encoded by the wild-type germline nucleic acid sequence of the subject;
(c) designing and/or selecting or having designed and/or selected (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture at least the tumor-associated mutations optionally wherein the polynucleotide regions of interest comprise at least 50 tumor-associated mutations;
(d) obtaining or having obtained sequencing data of cell-free DNA (cfDNA) from a pre-therapy sample from the subject, wherein the pre-therapy cfDNA was enriched prior to sequencing using the polynucleotide probes, and wherein the sequencing data comprises a target coverage of at least 50% of all polynucleotide regions of interest corresponding to the tumor-associated mutations and wherein the sequenced polynucleotide regions of interest comprise read depth of at least 1000×, optionally wherein the mean read coverage is mean duplex read coverage, and optionally wherein obtaining the sequencing data comprises collecting or having collected the pre-therapy sample from the subject, isolating or having isolated the pre-therapy cfDNA, enriching or having enriched the pre-therapy cfDNA, and/or sequencing or having sequenced the pre-therapy cfDNA;
(e) obtaining or having obtained sequencing data of cell-free DNA (cfDNA) from a post-therapy sample from the subject, optionally wherein the therapy comprises a cancer vaccine comprising the neoantigen or expression system encoding the same, wherein the post-therapy cfDNA was enriched prior to sequencing using the polynucleotide probes, and wherein the sequencing data comprises a target coverage of at least 50% of all polynucleotide regions of interest corresponding to the tumor-associated mutations and wherein the sequenced polynucleotide regions of interest comprise read depth of at least 1000×, optionally wherein the mean read coverage is mean duplex read coverage, and optionally wherein obtaining the sequencing data comprises collecting or having collected the post-therapy sample from the subject, isolating or having isolated the post-therapy cfDNA, enriching or having enriched the post-therapy cfDNA, and/or sequencing or having sequenced the post-therapy cfDNA; and
(f) determining or having determined the frequency of the tumor-associated mutations of the pre-therapy cfDNA relative to the post-therapy cfDNA to assess the efficacy of the therapy, optionally wherein at least the one or more tumor-associated mutations associated with the neoantigen is determined, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable,
optionally wherein the method comprises designing and/or selecting or having designed and/or selected a combination panel of a panel of tumor-informed polynucleotide probes and a panel of tumor-naïve polynucleotide probes, further optionally wherein the designed and/or selected combination panel comprises the panel of claim 1.

40-44. (canceled)

45. The method of claim 37, wherein the method comprises:
(A) one or more of the steps of:
(a) collecting or having collected the sample from the subject;
(b) isolating or having isolated the cfDNA;
(c) enriching or having enriched the cfDNA; or
(d) sequencing or having sequenced the cfDNA;
optionally each of the steps of:
(a) collecting or having collected the sample from the subject;
(b) isolating or having isolated the cfDNA;
(c) enriching or having enriched the cfDNA; and
(d) sequencing or having sequenced the cfDNA; and/or
(B) the steps of:
(a) obtaining or having obtained sequencing data of tumor-derived DNA from a cancer-diseased tissue from the subject, optionally wherein obtaining the sequencing data comprises collecting or having collected the cancer-diseased tissue, isolating or having isolated the tumor-derived DNA, and sequencing or having sequenced the tumor-derived DNA;
(b) determining or having determined one or more tumor-associated mutations relative to a wild-type germline nucleic acid sequence of the subject from the tumor-derived DNA sequencing data, optionally wherein one or more of the one or more tumor-associated mutations is associated with a neoantigen comprising at least one alteration that makes a peptide sequence encoded by the tumor-derived DNA distinct from the corresponding peptide sequence encoded by the wild-type germline nucleic acid sequence of the subject;
(c) designing and/or selecting or having designed and/or selected (1) a panel of tumor-informed polynucleotide probes; (2) a panel of tumor-naïve polynucleotide probes; and/or (3) a combination panel of a panel of tumor-informed polynucleotide probes and a panel of tumor-naïve polynucleotide probes, wherein the polynucleotide probes are configured to capture the polynucleotide regions of interest corresponding to the tumor-associated mutations optionally wherein the polynucleotide regions of interest comprise at least 50 tumor-associated mutations; and
(d) enriching or having enriched the cfDNA using the polynucleotide probes prior to sequencing.

46. (canceled)

47. The method of claim 37, wherein:
(I) the mean read depth comprises:
(A) at least 1500×, at least 2000×, at least 2500×, 3000×, at least 3500×, at least 4000×, at least 4500×, or at least 5000× mean read coverage;
(B) a range from 1000× to 5000× mean read coverage;
(C) a range from 1000× to 4000×, 1000× to 3000×, 1000× to 2000×, 2000× to 5000×, 2000× to 4000×, 2000× to 3000×, 3000× to 5000×, 3000× to 4000×, or 4000× to 5000× mean read coverage; and/or
(D) mean read duplex depth, and/or
(II) each of the polynucleotide regions of interest corresponding to the mutations present in the exome comprise a read depth of:
(A) at least 1000×; or
(B) at least 1000×, at least 1500×, at least 2000×, at least 2500×, 3000×, at least 3500×, at least 4000×, at least 4500×, or at least 5000×, and/or
(III) the target coverage comprises:
(A) at least 60%, at least 70%, at least 80%, or at least 90% of polynucleotide regions of interest corresponding to the mutations present in the exome of the cancer;
(B) at least 95%, at least 96%, at least 97%, at least 98%, at least 99%, at least 99.5%, at least 99.9%, or 100% of polynucleotide regions of interest corresponding to the mutations present in the exome of the cancer; and/or
(C) at least 95% of polynucleotide regions of interest corresponding to the mutations present in the exome of the cancer, and/or
(IV) the polynucleotide regions of interest comprise at least 50, at least 60, at least 70, at least 80, at least 90 mutations, at least 100, at least 150, at least 200, at least 250, at least 300, at least 400, at least 500, at least 600, at least 700, at least 800, at least 900, or at least 1000 mutations.

48-60. (canceled)

61. The method of claim 37, wherein the subject has been administered a therapy, optionally wherein the therapy comprises a cancer vaccine, further optionally wherein:
(A) the cancer vaccine comprises an epitope-encoding nucleic acid sequence encoding at least one of the mutations present in the exome of the cancer;
(B) the cancer vaccine comprises a self-amplifying alphavirus-based expression system; and/or
(C) the cancer vaccine comprises a chimpanzee adenovirus (ChAdV)-based expression system.

62-65. (canceled)

66. The method of claim 37, wherein the method comprises obtaining sequencing data of cfDNA from two or more samples from the subject;
optionally wherein the two or more samples are collected at different time points,
further optionally wherein the two or more samples are collected at different time points relative to administration of a therapy,
further optionally wherein a pre-therapy sample is collected prior to administration of the therapy and a post-therapy cfDNA is collected subsequent to administration of the therapy;
further optionally wherein the determining step comprises determining or having determined the frequency of the mutations of the pre-therapy cfDNA relative to the post-therapy cfDNA to assess the efficacy of the therapy, optionally wherein at least the one or more tumor-associated mutations associated with the neoantigen is determined, optionally wherein an increase in the frequency of the mutations in the post-therapy cfDNA relative to the pre-therapy cfDNA indicates an increased likelihood that tumor burden of the subject is increasing, and optionally wherein a decrease or maintenance of the frequency of the mutations in the post-therapy cfDNA relative to the pre-therapy cfDNA indicates an increased likelihood that tumor burden of the subject is decreasing or stable; further optionally wherein:
(A) an increase in the frequency of one or more of the mutations in the tumor-naïve panel in the post-therapy cfDNA relative to the pre-therapy cfDNA indicates a likelihood of an immune evasion mechanism tumor mutation;
(B) an increase in the frequency of the mutations in the post-therapy cfDNA relative to the pre-therapy cfDNA indicates an increased likelihood that tumor burden of the subject is increasing
(C) a decrease or maintenance of the frequency of the mutations in the post-therapy cfDNA relative to the pre-therapy cfDNA indicates an increased likelihood that tumor burden of the subject is decreasing or stable; optionally wherein the decrease comprises a Complete Response (CR) or a Partial Response (PR).

67-74. (canceled)

75. The method of claim 37, wherein the method further comprises administering a therapy to the subject following the assessment of the status of the cancer, optionally wherein:
(A) the assessment of the frequency of the mutations in the cfDNA indicates a likelihood the subject has or still has cancer; and/or
(B) the therapy comprises a cancer vaccine, optionally wherein the cancer vaccine comprises:
(a) an epitope-encoding nucleic acid sequence encoding at least one of the mutations present in the exome; and/or
(b) a self-amplifying alphavirus-based expression system or a chimpanzee adenovirus (ChAdV)-based expression system.

76-80. (canceled)

81. The method of claim 37, wherein;
(A) the collecting step comprises collecting a blood sample;
(B) the isolation step comprises centrifugation to separate cfDNA from cells and/or cellular debris;
(C) the isolation step comprises isolating cfDNA from whole blood, optionally wherein isolating cfDNA from whole blood comprises separating the plasma layer, buffy coat, and red blood cells, further optionally wherein the cfDNA is isolated from the plasma layer;
(D) the sequencing step comprises next generation sequencing (NGS) or Sanger sequencing; optionally wherein NGS comprises duplex sequencing, whole-exome sequencing, whole-genome sequencing, de novo sequencing, phased sequencing, targeted amplicon sequencing, or shotgun sequencing;
(E) the enrichment step comprises enriching the cfDNA for the polynucleotide regions of interest corresponding to the mutations present in the exome prior to sequencing, optionally wherein:
(a) the enrichment comprises the combination of the panel of tumor-informed polynucleotide probes and the panel of tumor-naïve polynucleotide probes, optionally wherein separate samples are separately enriched for each of the panel of tumor-informed polynucleotide probes and the panel of tumor-naïve polynucleotide probes, further optionally wherein the tumor-informed polynucleotide probes comprises each of the polynucleotide regions of interest corresponding to the mutations present in the exome;
(b) the tumor-informed polynucleotide probes comprises at least 50%, at least 60%, at least 70%, at least 80%, at least 90%, at least 95%, at least 96%, at least 97%, at least 98%, at least 99%, at least 99.5%, at least 99.9%, or 100% of polynucleotide regions of interest corresponding to the mutations present in the exome of the cancer; and/or
(c) the tumor-informed polynucleotide probes comprises at least 50, at least 60, at least 70, at least 80, at least 90 mutations, at least 100, at least 150, at least 200, at least 250, at least 300, at least 400, at least 500, at least 600, at least 700, at least 800, at least 900, or at least 1000 mutations, optionally the mutations present in the exome of the cancer; and/or
(F) the enrichment step comprises hybridizing one or more polynucleotide probes to the one or more polynucleotide regions of interest.

82-93. (canceled)

94. The panel of claim 1, wherein:
(A) the polynucleotide probes are 80 to 150 base pairs (bp) in length, optionally wherein the polynucleotide probes are 50-100, 50-150, 80 to 140, 80 to 130, 80 to 120, 80 to 110, 80 to 100, 80 to 90, 90 to 150, 90 to 140, 90 to 130, 90 to 120, 90 to 110, 90 to 100, 100 to 150, 100 to 140, 100 to 130, 100 to 120, 100 to 110, 110 to 150, 110 to 140, 110 to 130, 110 to 120, 120 to 150, 120 to 140, 120 to 130, 130 to 150, 130 to 140, 140 to 150, 50, 60, 70, 80, 90, 100, 110, 120, 130, 140, or 150 bp in length;
(B) the one or more polynucleotide probes are biotinylated;
(C) one or more of the mutations comprises a point mutation, a frameshift mutation, a non-frameshift mutation, a deletion mutation, an insertion mutation, a splice variant, a genomic rearrangement, a proteasome-generated spliced antigen, or combinations thereof;
(D) one or more of the mutations comprises at least one alteration that makes a peptide sequence encoded by the cfDNA distinct from the corresponding peptide sequence encoded by the wild-type germline nucleic acid sequence of the subject; and/or
(E) the one or more mutations consists of coding mutations comprising at least one alteration that makes a peptide sequence encoded by the cfDNA distinct from the corresponding peptide sequence encoded by the wild-type germline nucleic acid sequence of the subject.

95-96. (canceled)

97. The panel of claim 1, wherein the tumor-informed polynucleotide probes are designed or selected following sequencing of a tumor of the subject.

98. The panel of claim 97, wherein the tumor-informed polynucleotide probes are designed or selected following exome sequencing of the tumor of the subject and/or wherein the tumor-informed polynucleotide probes are designed or selected to target all mutations of the sequenced tumor.

99-104. (canceled)


SEQUENCE LISTING (6 sequences)

SEQ ID NO: 1 - DNA, 332 bp, synthetic construct
  source 1..332: mol_type = other DNA
cagaagcagaggggtcagggcgaagtcccagggccccaggcgtggctctcagggtctcaggccccgaaggcggtgtatggattcgtccagaggatgtatggctgcgacgtggggtcggactggcgcttcctccgcgggtaccaccagtacgcctacgacggcaagggcaggggatggaaccttccagaagtgggcggctgtggtggtgccttctggacaggagcagagatacacctgccatgtgcagcagggtgttgggcggaacagtggacacagctgtgctatggggtttctttccattggatgtattgagcatgcgatgggctgtttaa

SEQ ID NO: 2 - DNA, 332 bp, synthetic construct
  source 1..332: mol_type = other DNA
cagaagcagaggggtcagggcgaagtcccagggccccaggcgtggctctcagggtctcaggccccgaaggcggtgtatggattatccagatgatgtatggctgcgacgtggggtcggacgggcgcttcctccgcgggtaccggcaggacgcctacgacggcaaggacaggggatggaaccttccagaagtgggcgtctgtggtggtgccttctggacaggagcagagatacacctgccatgtgcagcattgggtgttgggcggaacagtggacgcagctgtgctatggggtttctttgcattggatgtattgagcatgcgatgggctgttta

SEQ ID NO: 3 - DNA, 332 bp, synthetic construct
  source 1..332: mol_type = other DNA
gatcaggacgaagtcccaggtcccggacggggctctcagggtctcaggctccgagggccgcgtctgcaatggggaggcgcagctacggctgcgacgtggggccggacgggcgcctcctccgcgggcataaccagtacgcctacgacggcaaggattacatcgccctgccttctggagaagagcagagatacacatgccatgtacagcatgaggggctgccgaagcccctcaccctgagatggggtaagggtggtgggctgtttagagtgtcatcacttaccatgactaaccagaatttgttcatgactgttgttttctgtagaatgagacag

SEQ ID NO: 4 - DNA, 334 bp, synthetic construct
  source 1..334: mol_type = other DNA
gatcaggacgaagtcccaggccccgggcggggctctcagggtctcaggctccgagggccgactctgcaatggggaggcgcagcgatgtatggctgcgacctggggcccgacgggcgcctcctccgcgggtataaccagttagcctacgacggcaaggattacatcgtggtggtgccttctggagaagagcagagatacacatgccatgtacagcatgaggggctgccgaagcccctcaccctgagatggttgggatgtttcgcgtgtgtggtgggctgttcagagtgtcatcacttaccatgactaaccagaatttgttcatgactgttgttgg

SEQ ID NO: 5 - DNA, 415 bp, synthetic construct
  source 1..415: mol_type = other DNA
ttattttgctggatgtagtttaatattacctgaggtaaggtaaggcaaagagtgggaggcagggagtccagttcagggacggggcccgggtcgccccgagtctcccggtctgagatccaccccgaggctgcggaacccgcccagaccctcgaccggagagagcccctgtctgaattttctgactcttcccgtcagaacacccaaagacacacgtgacccaccatcccgtctctgaccatgaggccacccgatggcggtgggcgtgtggaggagctcacccaccccataattcctcttgtcccacatctcctgcgggctctgaccaggtctttctccttctagggctcctcttctgctttggtctcctgtcctctctcccttccctgattccagtaatcttcgtgctgactccaat

SEQ ID NO: 6 - DNA, 415 bp, synthetic construct
  source 1..415: mol_type = other DNA
ttattttgctggatgtagtttaatattacctgaggtgaggtaaggtaaggcaaagggtgggaggcagggagtccagttcagggggacggcccgggtcgccccgagtctccccgtctgagatccaccccaaggtggatctgcggaacccgcccagaccctcgaccgggatgcaaagtgtctgaattttctgactcttcccgtcagaacccccaaagacacacgtgacccaccaccccctctctgaccatggctgcgtgtaagtgatggcggcgggcgtgtggaggagctcacctactccataattcctcttgtcccacatctcctgcgggctccattcagctcctgccctccttctagggctcctcttctgctttggtctcctgccctctctcccttccctgattccagtgatctt